(19) United States
(12) Patent Application Publication
(10) Pub. No.: US 2008/0182851 A1
(43) Pub. Date: Jul. 31, 2008

(54) ACETYLENE DERIVATIVES AS STEAROYL COA DESATURASE INHIBITORS

(75) Inventors: Abraham Thomas, Navi Mumbai (IN); V.S. Prasada Rao Lingam, Navi Mumbai (IN); Shantaram Kashinath Phatangare, Navi Mumbai (IN); Neelima Khairatkar-Joshi, Thane (IN); Daisy Manish Shah, Dadar (IN); Deepak Vitthal Ukirde, Pune (IN); Dattaguru Anandrao More, Kolhapur (IN)

Correspondence Address: DARBY & DARBY P.C., P.O. BOX 770, Church Street Station, New York, NY, 10008-0770, US

(73) Assignee: Glenmark Pharmaceuticals S.A., La Chaux-de-Fonds (CH)

(21) Appl. No.: 11/942,522

(22) Filed: Nov. 19, 2007

Related U.S. Application Data
(60) Provisional application No. 60/954,108, filed on Aug. 6, 2007.

(30) Foreign Application Priority Data
Nov. 20, 2006 (IN) 1917/MUM/2006
Dec. 29, 2006 (IN) 2175/MUM/2006
Jul. 17, 2007 (IN) 1375/MUM/2007

(51) Int. Cl. A61K 31/501; C07D 401/04; A61K 31/496; A61P 3/04; A61P 3/08; A61P 3/10; A61P 3/00; C07D 403/04

(52) U.S. Cl. 514/252.02; 544/360; 514/253.01; 544/238

(57) ABSTRACT

The present invention provides Stearoyl CoA Desaturase (SCD) inhibitors. In particular, compounds described herein are useful for treating or preventing diseases, conditions and/or disorders modulated by Stearoyl CoA Desaturase 1 (SCD1) inhibitors. Also provided herein are processes for preparing compounds described herein, intermediates used in their synthesis, pharmaceutical compositions thereof, and methods for treating or preventing diseases, conditions and/or disorders modulated by Stearoyl CoA Desaturase (SCD) inhibitors.

This application claims the benefit of Indian Patent Application No. 1917/MUM/2006 filed on Nov. 20, 2006, 2175/MUM/2006 filed on Dec. 29, 2006 and 1375/MUM/2007 filed on Jul. 17, 2007, and U.S. Provisional Application No. 60/954,108, filed on Aug. 6, 2007, all of which are hereby incorporated by reference

### FIELD OF THE INVENTION

The present invention provides Stearoyl CoA Desaturase (SCD) inhibitors. In particular, compounds described herein are useful for treating or preventing diseases, conditions and/or disorders modulated by Stearoyl CoA Desaturase 1 (SCD1) inhibitors. Also provided herein are processes for preparing compounds described herein, intermediates used in their synthesis, pharmaceutical compositions thereof, and methods for treating or preventing diseases, conditions and/or disorders modulated by Stearoyl CoA Desaturase (SCD) inhibitors.

### BACKGROUND OF THE INVENTION

Metabolic energy balance is important for well being which is maintained by appropriate adjustment between energy intake and energy expenditure. Primary defects in energy balance produce obesity. Over the past few years there has been a sharp increase in obesity in many countries. Obesity is a principal cause of morbidity and mortality mainly because it increases risk for other conditions that shorten life, including diabetes, insulin resistance, coronary artery disease, hypertension and non-alcoholic fatty liver disease collectively known as metabolic syndrome (J. Am. Med. Assoc., 288, 1723-1727 (2002)). Obesity has been identified as an independent risk factor for the development of type 2 diabetes.

Although the exact etiology of many events underlying obesity is not very well known, typically obesity is manifested by an increase in plasma free fatty acids and excessive lipid accumulation in some organs. Abnormal lipid metabolism in obese subjects results in accumulation of significant amounts of fat in liver, adipose tissue, muscle and other peripheral tissues which sets in insulin resistance (Obesity Reviews, 6, 169-174 (2005)). In the liver, fatty acids accumulate causing an increase in hepatic lipid content or get packaged into the very low density lipoprotein for export to other peripheral tissues. Liver steatosis associated with obesity can also result from an enhanced rate of de novo fatty acid synthesis and/or dysregulation of intracellular lipid partitioning in which fatty acid oxidation is impaired and its esterification enhanced. Lipid abnormalities in obese subjects, in particular hypertriglyceridemia, low HDL cholesterol and altered LDL cholesterol particle size, are atherogenic. The dyslipidemic state initiates a cascade of events including release of proinflammatory adipokines which induces a proinflammatory state that drives pathogenesis of atherosclerosis. Increased release of proinflammatory adipokines also increases fibrinogen and plasminogen activator inhibitor levels thereby increasing risk for arterial thrombosis. Several studies show that even modest wait gain can precipitate the onset of hypertension (Ann. Rev. Med., 56, 45-62 (2005)). Hence obesity alone can drive all aspects of the metabolic syndrome. It is believed that effective treatment of obesity could lead to prevention and control of metabolic syndrome (Obesity Reviews, 6, 169-174 (2005)).

Stearoyl-CoA desaturase 1 (SCD1) has been shown to be a key enzyme that plays a crucial role in lipid metabolism and body weight control (Science, 297, 240-243 (2002); Obesity Reviews, 6, 169-174 (2005); J. Clinical Investigation, 1-9 (2005)). SCD1 is a central lipogenic enzyme catalyzing the biosynthesis of monounsaturated fatty acids from saturated fatty acids by addition of a cis double bond between carbon 9 and carbon 10 (PNAS, 71, 4565-4569 (1974); J Biol Chem., 251, 5095-5103 (1976)). SCD1 has two preferred substrates, palmitoyl and stearoyl CoA, which are desaturated to palmitoleoyl and oleoyl CoA respectively (J Biol Chem., 251, 5095-5103 (1976)). Oleate is found to be the major monounsaturated fatty acid of membrane phospholipids, triglycerides, cholesterol esters, wax esters and alkyl-1,2-diacylglycerol. The ratio of stearate to oleate is one of the factors influencing membrane fluidity and its alteration is important in diseases like aging, cancer, diabetes, obesity, and neurological, vascular and heart diseases (Biochem. Biophys. Acta., 431, 469-480 (1976); J. Biol. Chem., 268, 6823-6826 (1993); Diabetes, 40, 280-289 (1991); Neurochem Res., 26, 771-782 (1994); Arthritis Rheum., 43, 894-900 (2000); Cancer Lett., 173, 139-144 (2001)).

The role of SCD1 in regulation of body weight is well discussed in the literature. Robust up-regulation of SCD1 expression and/or activity is observed in obese experimental animals (Science, 297, 240-243 (2002)), fat chickens (Am Soc Nutri Scie., 249-256 (1997)) and obese human subjects (Cell Metab., 2, 251-61 (2005)) compared to their lean counterparts. In chickens, the fat chickens have higher hepatic delta-9 desaturase activity and higher plasma triglyceride compared to lean birds. Inhibition of delta-9 desaturase by a mixture of cyclopropenic fatty acids resulted in reduced triglyceride formation in vitro in hepatocytes isolated from the fat chickens (Am Soc Nutri Scie., 249-256 (1997)). SCD1 over activity leads to weight gain and its deficiency leads to leanness. SCD1 deficiency either directly or indirectly induces a signal that partitions fatty acids towards oxidation rather than synthesis. Asebia mice with a natural mutation in the SCD1 gene manifest defective cholesterol ester and triglyceride synthesis and are lean and hypermetabolic (J. Biol. Chem., 275, 30132-30138 (2000); Science, 297, 240-243 (2002)). Laboratory mice with targeted disruption in the SCD1 gene are resistant to diet-induced obesity and have reduced body adiposity, liver lipid accumulation and postprandial plasma insulin and glucose levels, with concomitant increase in the metabolic rate, thermogenesis and insulin sensitivity (J Nutr., 131, 2260-2268 (2001); PNAS, 99, 11482-11486 (2002)). SCD1 is documented as a key enzyme in regulating hepatic lipogenesis and lipid oxidation and therapeutic manipulation of SCD can be of benefit in treatment of obesity and metabolic syndrome (Obesi Reviews, 6, 169-174 (2005); Curr Drug Targets Immune Endocr Metabol Disord., 3, 271-280 (2003)).

Several studies report inhibition of SCD1 expression and activity by different agents such as thia-fatty acids like 9-thiastearic acid, cyclopropenoid fatty acids like sterculic acid and certain conjugated linoleic acid isomers. Trans-10, cis-12 isomer of conjugated linolenic acid inhibits SCD1 expression as well as desaturase activity in vitro (Biochim Biophys Acta., 1486 (2-3), 285-292 (2000); Biochem Biophys Res Commun., 284(3), 689-693 (2001)). Conjugated linoleic acid (CLA) administration through feed reduces body fat and increases lean body mass in several animal species (Lipids, 32, 853-858 (1997); FASEB, 12, A836 (1998); Lipids, 34, 243-248 (1999)). Sterculic acid (8-(2-octylcyclopropenyl) octanoic acid) and malvalic acid (7-(2-octylcyclopropenyl)heptanoic acid) are C18 and C16 derivatives of sterculoyl- and malvaloyl fatty acids, respectively and inhibit SCD enzymatic activity by direct interaction with the enzyme. However all these agents are weak and non-specific inhibitors of SCD1. SCD1 antisense oligonucleotide inhibitors specifically reduce SCD1 expression thereby reducing fatty acid synthesis and secretion, body adiposity, hepatomegaly, steatosis and prevent obesity in mice by improving energy balance (J Clinical Investigation, F 1-9 (2005)).

U.S. Publication No. 2006/009459 and PCT Publication Nos. WO 2005/011653, 2005/01164, 2005/011655, 2005/011656 and 2005/011657 disclose certain pyridazine derivatives, pyridyl derivatives, and piperazine derivatives and their use for inhibiting human stearoyl-CoA desaturase (hSCD) activity. U.S. Publication No. 2004/072877 is directed to a method for increasing insulin sensitivity by reducing stearoyl-CoA desaturase 1 (SCD1) activity in a subject sufficiently to increase insulin sensitivity.

There is a need for safer and more effective therapeutic treatments for diseases, conditions and/or disorders modulated by SCD. In particular, there is a need for novel compounds that are used for treating obesity, diabetes, cardiovascular disease and complication thereof.

### SUMMARY OF THE INVENTION

The present invention provides acetylene derivatives as SCD inhibitors, which are useful in the treatment of diseases, conditions or disorders modulated by SCD (and in particular SCD1), and processes for the synthesis of these compounds. Pharmaceutically acceptable salts, solvates, prodrugs, stereoisomers and N-oxides of these compounds having the same type of activity are also provided. Pharmaceutical compositions containing compounds described herein optionally together with one or more pharmaceutically acceptable excipients (e.g., carriers or diluents), which are useful for treating diseases, conditions or disorders modulated by SCD are further provided.

In one aspect, the compound of the present invention is

A-U-B′-≡-Q Formula I

or a pharmaceutically acceptable salt thereof, solvate thereof, prodrug thereof, stereoisomer thereof or N-oxide thereof,
wherein

A is R′W—;

R′ is selected from substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heteroarylalkyl, substituted or unsubstituted heterocyclic and substituted or unsubstituted heterocyclylalkyl;

W is selected from (CR₁R₂)ₚ, C(═Y), C(═Y)O, OC(═Y), O, CONR₁S(O)ᵣ, S(O)ᵣNR₁, N(R₁)—(CH₂)ₙO, NR₁ and N(R₁)—C(═Y)NR₂;

Q is selected from hydrogen, hydroxyl, substituted or unsubstituted alkyl (e.g., substituted or unsubstituted hydroxyalkyl), substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heteroarylalkyl, substituted or unsubstituted heterocyclic, substituted or unsubstituted heterocyclylalkyl, (CR₁R₂)ₙOR₅, COR₁, COOR₁, CONR₁R₂, S(O)ᵣNR₁R₂, NR₁R₂, (CH₂)ₙNR₁R₂, (CH₂)ₙCHR₁R₂, (CR₁R₂)NR₅R₆, (CR₁R₂)NR₅CONR₆R₇, (CH₂)ₙNHCOR₁ and (CH₂)ₙNHSO₂R₁;

U is selected from a bond and

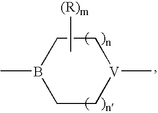

wherein V is CR or N and B is CR or N, or B together with an adjacent ring carbon atom and A form a ring selected from

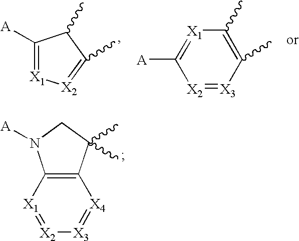

B′ is selected from

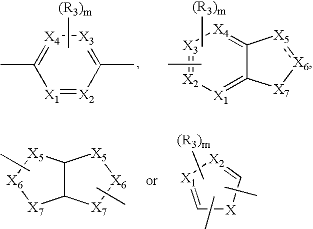

each occurrence of R is independently selected from hydrogen, hydroxyl, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heterocyclyl, substituted or unsubstituted heterocyclylalkyl or substituted or unsubstituted heteroarylalkyl;

each occurrence of m is independently an integer 0-4; each occurrence of n, n′, and r are independently 0, 1 or 2; p is 0, 1, 2, 3 or 4;

each occurrence of R₁, R₂, R₅, R₆, and R₇ may be same or different and are independently hydrogen, hydroxyl, halogen, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heterocyclic, substituted or unsubstituted heterocyclylalkyl or substituted or unsubstituted heteroarylalkyl, or, when R₁ and R₂ are attached to a common atom, form with the common atom a 3-7 membered heterocyclyl;

X₁ to X₄ are independently N or CR;

each occurrence of X and X₅ to X₇ are independently CHR₄, CO, CS, O, S(O)ᵣ, N or NR₄;

each occurrence of R₄ is independently selected from hydrogen, hydroxyl, substituted or unsubstituted alkyl (e.g., substituted or unsubstituted hydroxylalkyl), substituted or unsubstituted cycloalkyl, substituted or unsubstituted aryl, substituted or unsubstituted cycloalkyl, substituted or unsubstituted heteroaryl and substituted or unsubstituted heterocyclyl;

each occurrence of R₃ is selected from hydrogen, nitro, cyano, halogen, COR₁, substituted or unsubstituted alkyl, substituted or unsubstituted aryl, substituted or unsubstituted heteroaryl, substituted or unsubstituted alkoxy, COOR₁, CONR₁R₂, S(O)ᵣR₁, S(O)ᵣNR₁R₂ and NR₁R₂; and

each occurrence of Y is O or S.

It is to be understood that the embodiments below are illustrative of the present invention and are not intended to limit the claims to the specific embodiments exemplified.

One preferred embodiment is a compound of formula I, wherein R′ is substituted or unsubstituted aryl, substituted or unsubstituted heteroaryl, or substituted or unsubstituted cycloalkyl.

Another preferred embodiment is a compound of formula I, wherein W is CH₂, CO, CS, O, NH(CH₂)₂O or NH.

Another preferred embodiment is a compound of formula I, wherein B is CR or N.

Another preferred embodiment is a compound of formula I, wherein V is N.

Another preferred embodiment is a compound of formula I, wherein B and V are N.

Another preferred embodiment is a compound of formula I, wherein R is H or OH.

Another preferred embodiment is a compound of formula I, wherein X₁ is N.

Another preferred embodiment is a compound of formula I, wherein X is S.

Another preferred embodiment is a compound of formula I, wherein X₂-X₄ are independently CR or N.

Another preferred embodiment is a compound of formula I, wherein n is 0 or 1 and n′ is 0 or 1.

Another preferred embodiment is a compound of formula I, wherein B′ is selected from

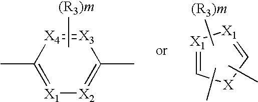

Another preferred embodiment is a compound of formula I, wherein Q is selected from substituted or unsubstituted alkyl (e.g., substituted or unsubstituted hydroxyalkyl), substituted or unsubstituted cycloalkyl, substituted or unsubstituted aryl, substituted or unsubstituted arylalkyl, substituted or unsubstituted heteroaryl, substituted or unsubstituted heterocyclic, (CR₁R₂)ₙOR₅, (CH₂)ₙNHCOR₁ and (CH₂)ₙNHSO₂R₁.

According to another embodiment,

R′ is selected from substituted or unsubstituted aryl, substituted or unsubstituted heteroaryl and substituted or unsubstituted cycloalkyl;

W is selected from CH₂, CO, O, NH(CH₂)₂O or NH;

B′ is selected from

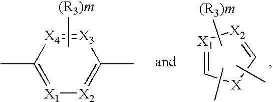

wherein X₁ is N; X₂-X₄ are independently CR or N; X is S; m is an integer 0-4; p is 0, 1, 2, 3 or 4; R₃ is hydrogen;

U is selected from bond and

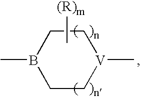

wherein B is CH, C(OH) or N, V is N, R is hydrogen, and n and n′ are independently 0 or 1;

Q is selected from substituted or unsubstituted alkyl, substituted or unsubstituted cycloalkyl, substituted or unsubstituted cycloalkylalkyl, substituted or unsubstituted aryl, substituted or unsubstituted arylalkyl, substituted or unsubstituted heteroaryl, substituted or unsubstituted heterocyclic, (CR₁R₂)ₙOR₅, (CR₁R₂)ₙNR₅COR₆R₇, (CH₂)ₙNHCOR₁ and (CH₂)ₙNHSO₂R₁; wherein R₁, R₂, R₅, R₆, and R_{c} independently are selected from hydrogen, substituted or unsubstituted alkyl, substituted or unsubstituted aryl, substituted or unsubstituted heteroaryl and substituted or unsubstituted cycloalkyl.

According to yet another embodiment,

R′ is selected from 2-trifluoromethylphenyl, 2,5-dichlorophenyl, 5-trifluoromethylpyridinyl, cyclopentyl, cyclopropyl, cyclohexylmethyl, 2-fluorophenyl, phenyl, 2-fluorophenyl, 4-bromo-2-fluorophenyl, 2-cyanophenyl and 3-pyridyl;

W is selected from CH₂, CO, O, NH(CH₂)₂O or NH; and

Q is selected from CH₂OH, C(CH₃)₂OH, substituted or unsubstituted cycloalkyl, C(OH)CH₂CH₃, (CH₂)OR₁, substituted or unsubstituted cycloalkylalkyl, substituted or unsubstituted arylalkyl, (CH₂)ₙNHSO₂R₁, (CH₂)ₙNHCOR₁, (CH₂)₂CH₃, C(CH₃)₃, substituted or unsubstituted aryl, substituted or unsubstituted heteroaryl, and substituted or unsubstituted heterocyclic.

According to one preferred embodiment of the present invention, the SCD1 inhibitor is selected from

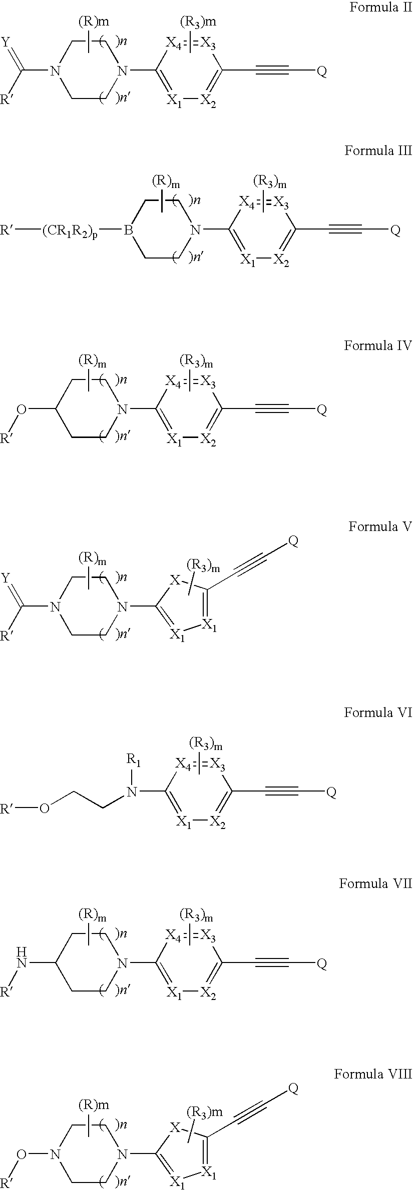

or a pharmaceutically acceptable salt thereof, solvate thereof, stereoisomer thereof, prodrug thereof, or N-oxide thereof,
wherein:

R′ is selected from substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heteroarylalkyl, substituted or unsubstituted heterocyclic and substituted or unsubstituted heterocyclylalkyl;

R is selected from hydrogen, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heterocyclyl, substituted or unsubstituted heterocyclylalkyl and substituted or unsubstituted heteroarylalkyl;

Q is selected from hydrogen, hydroxyl, substituted or unsubstituted alkyl (e.g., substituted or unsubstituted hydroxyalkyl), substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heteroarylalkyl, substituted or unsubstituted heterocyclic, substituted or unsubstituted heterocyclylalkyl, (CR₁R₂)ₙOR₅, COR₁, COOR₁, CONR₁R₂, S(O)ᵣNR₁R₂, NR₁R₂, (CH₂)ₙNR₁R₂, (CH₂)ₙCHR₁R₂, (CR₁R₂)NR₅R₆, (CR₁R₂)NCOR₅R₆, (CH₂)ₙNHCOR₁ or (CH₂)ₙNHSO₂R₁;

R₃ is selected from hydrogen, nitro, cyano, halogen, COR₁, substituted or unsubstituted alkyl, substituted or unsubstituted aryl, substituted or unsubstituted heteroaryl, substituted or unsubstituted alkoxy, COOR₁, CONR₁R₂, S(O)ᵣR₁, S(O)ᵣNR₁R₂ or NR₁R₂;

each occurrence of m is independently an integer 0-4; n and n′ are independently 0, 1 or 2; Y is O or S; p is 0, 1, 2, 3 or 4;

each occurrence of R₁, R₂, R₅, and R₆ may be same or different and are independently hydrogen, halogen, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heterocyclic, substituted or unsubstituted heterocyclylalkyl or substituted or unsubstituted heteroarylalkyl, or when R₁ and R₂ are attached to a common atom, form with the common atom a substituted or unsubstituted 3-7 membered heterocyclyl;

each occurrence of X₁ to X₄ are independently N or CR; each occurrence of r is independently an integer 0-2; n″ is an integer 0-6.

A preferred embodiment is a compound of formulae II-VIII, wherein R′ is substituted or unsubstituted aryl, substituted or unsubstituted heteroaryl, or substituted or unsubstituted cycloalkyl.

Another preferred embodiment is a compound of formulae II and V, wherein Y is O.

Another preferred embodiment is a compound of formulae II-VIII, wherein R is H or OH and R₃ is H.

Another preferred embodiment is a compound of formulae II-VIII, wherein n is 0 or 1 and n′ is 0 or 1.

Yet another embodiment is a compound of formulae II-VIII, wherein X₁ is N and X₂-X₄ are independently N or CR.

Yet another embodiment is a compound of formulae II-VIII, wherein Q is selected from substituted or unsubstituted alkyl (e.g., substituted or unsubstituted hydroxyalkyl), substituted or unsubstituted cycloalkyl, substituted or unsubstituted aryl, substituted or unsubstituted arylalkyl, substituted or unsubstituted heteroaryl, substituted or unsubstituted heterocyclic, (CR₁R₂)ₙOR₅, (CH₂)ₙNHCOR₁ and (CH₂)ₙNHSO₂R₁.

Yet another embodiment is a compound of formulae II-VI, wherein Q is selected from substituted or unsubstituted aryl or substituted or unsubstituted heteroaryl.

Yet another embodiment is a compound of formula II

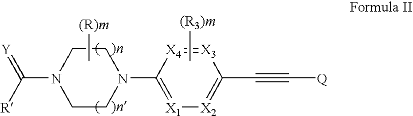

wherein

Y is O;

n and n′ are l, m at each occurrence is 0;

X¹, X³, and X⁴ are CH, and X² is CH or N;

R′ is phenyl mono- or di-substituted with substituents selected from halogen (e.g., chloro) and —CF₃; and

Q is phenyl substituted with hydroxyl. R′ is preferably 2-trifluoromethylphenyl or 2,5-dichlorophenyl. Q is preferably 3-hydroxyphenyl or 4-hydroxyphenyl and more preferably 3-hydroxyphenyl.

Yet another embodiment is a compound of formula III

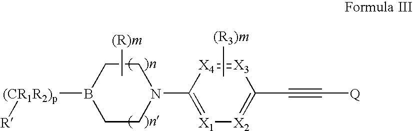

wherein

R′ is substituted or unsubstituted cycloalkyl or substituted or unsubstituted aryl;

each occurrence of m is 0;

n, n′, and p are 1;

B is C(OH) or N;

R₁ and R₂ are hydrogen;

X₁ and X₂ are N and X₃ and X₄ are CH; and

Q is phenyl substituted with —O—R⁴ where R⁴ is hydrogen or C(O)(C₁-C₆ alkyl). Preferably, R′ is cyclohexyl or phenyl mono- or di-substituted with halogen. More preferably, R′ is 2-fluorophenyl or 2,5-dichlorophenyl. B is preferably C(OH).

Yet another embodiment is a compound of formula IV

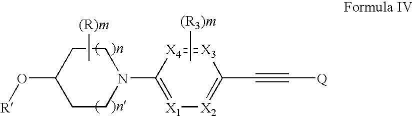

wherein

R′ is phenyl mono- or di-substituted with halogen;

each occurrence of m is 0;

n and n′ are 1;

X¹ is N, X⁴ is CH, and one of X² and X³ is N and the other is CH;

Q is phenyl substituted with —O—R⁴;

R⁴ is hydrogen, an alkali metal (e.g., potassium), or —(CH₂)_{q}C(O)R⁵; and

R⁵ is substituted or unsubstituted C₁-C₆ alkyl, substituted or unsubstituted heterocyclic, or substituted or unsubstituted heteroaryl. Preferably, R⁴ is hydrogen, an alkali metal (e.g., potassium), C(O)(C₁-C₆ alkyl) (e.g., C(O)CH₃), C(O)R⁵ (where R is heteroaryl (such as furoate)), or —(CH₂)ᵣ—R⁵ (where R is heterocyclic (such as 4-piperidin-1-yl or morpholine) and r is 1-3). More preferably, R′ is 2-fluorophenyl or 2,5-dichlorophenyl. More preferably, Q is 3-hydroxyphenyl or 4-hydroxyphenyl.

Representative examples of compounds of the present invention are provided below (Also shown in Table 1-6). These compounds are illustrative in nature only and do not limit the scope of the invention.
4-[5-(3-Hydroxy-1-propynyl)-2-pyridyl]piperazino-2-trifluoromethylphenyl methanone (Compound No. 1),
4-[5-(3-Hydroxy-1-propynyl)-2-pyridyl]piperazino-2,5-dichlorophenylmethanone (Compound No. 2),
4-[6-(3-Hydroxy-1-propynyl)-3-pyridazinyl]piperazino-2-trifluoromethylphenyl-methanone (Compound No. 3),
4-[5-(3-Hydroxy-3-methyl-1-butynyl)-2-pyrimidinyl]piperazino-2-trifluoromethylphenyl-methanone (Compound No. 4),
4-{5-[2-(1-Hydroxycyclopentyl)-1-ethynyl]-2-pyridyl}piperazino-2-trifluoromethyl Phenylmethanone (Compound No. 5),
4-[5-(3-Hydroxy-1-pentynyl)-2-pyridyl]piperazino-2-trifluoromethylphenyl methanone (Compound No. 6),
4-[5-{3-Hydroxy-3-(1-adamantyl)-1-propynyl}-2-pyridyl]piperazino-2-trifluoromethyl-phenylmethanone (Compound No. 7),
4-[5-(3-Hydroxy-3-phenyl-1-propynyl}-2-pyridyl}piperazino-2-trifluoromethylphenyl-methanone (Compound No. 8),
4-[5-(3-Cyclopentyloxy-1-propynyl)-2-pyridyl]piperazino-2-trifluoromethylphenyl-methanone (Compound No. 9),
4-{4-[3-(4-tert-Butylphenoxy)-1-propynyl]-2-pyridyl}piperazino-2-trifluoromethyl-phenylmethanone (Compound No. 10),
4-[5-(3-(4-Fluorophenoxy)-1-propynyl)-2-pyridyl]piperazino-2-trifluoromethylphenyl-methanone (Compound No. 11),
6-(3-{6-[4-(2-Trifluoromethylbenzoyl)piperazino]-3-pyridyl}-2-propynyloxy)nicotino-nitrile (Compound No. 12),
4-{5-[3-(4-Hydroxyphenoxy)-1-propynyl]-2-pyridyl}piperazino-2-trifluoromethyl-methanone (Compound No. 13),
1-{5-[3-(4-Fluorophenoxy)prop-1-yn-1-yl]-2-pyridyl}-4-(5-trifluoromethylpyridin-2-yl)piperazine (Compound No. 14),
N1-(3-{6-[4-(2-Trifluoromethylbenzoyl)piperazino]-3-pyridyl}-2-propynyl)acetamide (Compound No. 15),
N1-(3-{6-[4-(2-Trifluoromethylbenzoyl)piperazino]-3-pyridyl}-2-propynyl)-1-butane sulfonamide (Compound No. 16),
4-[5-(1-Pentynyl)-2-pyridyl]piperazino-2-trifluoromethylphenylmethanone (Compound No. 17),
4-[5-(3,3-Dimethyl-1-butynyl)-2-pyridyl]piperazino-2-trifluoromethylphenyl methanone (Compound No. 18),
2,5-Dichlorophenyl-4-[5-(3,3-dimethyl-1-butynyl)-2-pyridyl]piperazinomethanone (Compound No. 19),
4-[5-(2-Phenyl-1-ethynyl)-2-pyridyl]piperazino-2-trifluoromethylphenylmethanone (Compound No. 20),
2,5-Dichlorophenyl-4-[5-(2-phenyl-1-ethynyl)-2-pyridyl]piperazinomethanone (Compound No. 21),
4-(2-{4-[4-(2-Trifluoromethylbenzoyl)piperazino]pyridinyl-1-ethynyl)phenyl acetate (Compound No. 22),
4-{5-[2-(4-Hydroxyphenyl)-1-ethynyl]-2-pyridyl}piperazino-2-trifluoromethylphenyl methanone (Compound No. 23),
1-{5-[(3-Fluoro-4-hydroxyphenyl)ethynyl]-2-pyridyl}piperazin-4-yl-(2-trifluoromethylphenyl)methanone (Compound No. 24),
4-{5-[2-(3-Hydroxyphenyl)-1-ethynyl]-2-pyridyl}piperazino-2-trifluoromethylphenyl Methanone (Compound No. 25),
Ethyl-2-[3-(2-{6-[4-(2-trifluoromethylbenzoyl)piperazino]-3-pyridyl}-1-ethynyl)-phenoxy]acetate (Compound No. 26),
2-[3-(2-{6-[4-(2-Trifluoromethylbenzoyl)piperazino]-3-pyridyl}-1-ethynyl)phenoxy]-acetic acid (Compound No. 27),
2,5-Dichlorophenyl-4-{5-[2-(3-hydroxy-1-pentynyl}-1-ethynyl]-2-pyridyl}piperazino Methanone (Compound No. 28),
2-(2-{4-[4-(2-Trifluoromethylbenzoyl)piperazino]pyridinyl-1-ethynyl)phenyl acetate (Compound No. 29),
4-{5-[2-(4-Methoxyphenyl)-1-ethynyl]-2-pyridyl}piperazino-2-trifluoromethylphenyl methanone (Compound No. 30),
2,5-Dichlorophenyl-4-{5-[2-(3-methoxyphenyl)-1-ethynyl]-2-pyridyl}piperazino-methanone (Compound No. 31),
Methyl-4-(2-{6-[4-(2-trifluoromethylbenzoyl)piperazino]-2-pyridyl-1-ethynyl)benzoate (Compound No. 32),
4-{5-[2-(3-Hydroxymethylphenyl)-1-ethynyl]-2-pyridyl}piperazino-2-trifluoromethyl-phenylmethanone (Compound No. 33),
Ethyl-2-methylcarbonyloxy-5-(2-{6-[4-(2-trifluoromethylbenzoyl)piperazino-3-pyridyl}-1-ethynyl)benzoate (Compound No. 34),
2-Hydroxy-5-(2-{6-[4-(2-trifluoromethylbenzoyl)piperazino]-3-pyridyl}-1-ethynyl)-benzoic acid (Compound No. 35),
N1-[3-(2-{6-[4-(2-Trifluoromethylbenzoyl)piperazino]-3-pyridyl}-1-ethynyl)phenyl]Acetamide (Compound No. 36),
{4-[6-[4-(2-Trifluoromethylbenzoyl)piperazin-1-yl]pyridazin-3-yl]ethynyl}phenol (Compound No. 37),
4-{6-[2-(3-Hydroxyphenyl)-1-ethynyl]-3-pyridazinyl}piperazino-2-trifluoromethyl-phenylmethanone (Compound No. 38),
4-{5-[2-(4-Fluorophenyl)-1-ethynyl]-2-pyridyl}piperazino-2-trifluoromethylphenyl methanone (Compound No. 39),
4-{6-[2-(3,4-Difluorophenyl)-1-ethynyl]-3-pyridazinyl}piperazino-2(trifluoromethyl)-phenylmethanone (Compound No. 40),
2-Trifluoromethylphenyl-4-{6-[2-(4-trifluoromethylphenyl)-1-ethynyl]-3-pyridazinyl}-piperazinomethanone (Compound No. 41),
4-{5-[2-(4-Hydroxyphenyl)-1-ethynyl]-2-pyrimidinyl}piperazino-2-trifluoromethyl-phenylmethanone (Compound No. 42),
4-{5-[2-(3-Hydroxyphenyl)-1-ethynyl]-3-pyrimidinyl}piperazino-2-trifluoromethyl-phenylmethanone (Compound No. 43),
Ethyl 5-(2-{6-[4-(2-trifluoromethylbenzoyl)piperazino]-2-pyridyl-1-ethynyl)nicotinate (Compound No. 44),
4-{5-[(2-Pyrazinyl-1-ethynyl)-2-pyridyl]}piperazino-2-trifluoromethylphenyl methanone (Compound No. 45),
2,5-Dichlorophenyl-4-{5-(2-pyrimidinyl)-1-ethynyl]-2-pyridyl}piperazinomethanone (Compound No. 46),
4-{5-[2-(1-Butyl-1H-2-imidazolyl)-1-ethynyl]-2-pyridyl}piperazino-2-trifluoromethyl-phenylmethanone (Compound No. 47),
4-{5-[2-(1-(3-Methylbutyl)-1H-2-imidazolyl)-1-ethynyl]-2-pyridyl}piperazino-2-trifluoro-methylphenylmethanone (Compound No. 48),
4-{5-[2-(1H-5-Indolyl)-1-ethynyl]-2-pyridyl}piperazino-2-trifluoromethylphenyl-methanone (Compound No. 49),
4-{5-[2-(1H-5-Indolyl)-1-ethynyl]-2-pyrimidinyl}piperazino-2-trifluoromethylphenyl-methanone (Compound No. 50),
4-{5-[2-(4-(1,1-Dioxidoisothiazolidine-2-yl)phenyl)-1-ethynyl]-2-pyrimidinyl}piperazino-2-trifluoromethylphenylmethanone (Compound No. 51),
4-{5-[2-(4-(1H-1-Azolyl)phenyl)-1-ethynyl]-2-pyrimidinyl}piperazino-2-trifluoro-methylphenylmethanone (Compound No. 52),
4-(2-{2-[4-(2-Trifluoromethylbenzoyl)piperazino]-1,3-thiazol-5-yl}-1-ethynyl)phenyl acetate (Compound No. 53),
3-({6-[4-(Cyclopentylcarbonyl)piperazin-1-yl]pyridazin-3-yl}ethynyl)benzonitrile (Compound No. 54),
3-({6-[4-(Cyclopropylmethyl)piperazin-1-yl]pyridazin-3-yl}ethynyl)phenol (Compound No. 55),
3-([6-{(4-Cyclohexylmethyl)piperazin-1-yl]pyridazin-3-yl}ethynyl)phenyl acetate (Compound No. 56),
3-({6-[4-(Cyclohexylmethyl)piperazin-1-yl]pyridazin-3-yl}ethynyl)phenol (Compound No. 57),
3-{4-[(2-Fluorobenzyl)piperazin-1-yl]-6-(tetrahydro-2H-pyran-2-ylethynyl)}pyridazine (Compound No. 58),
4-[{6-[4-(2-Fluorobenzyl)piperazin-1-yl]pyridazin-3-yl}ethynyl]phenyl acetate (Compound No. 59),
3-({6-[4-(2-Fluorobenzyl)piperazin-1-yl]pyridazin-3-yl}ethynyl)phenol (Compound No. 60),
4-{[6-(4-Benzyl-4-hydroxypiperidin-1-yl)pyridazin-3-yl]ethynyl}phenyl acetate (Compound No. 61),
4-Benzyl-1-{6-[(4-hydroxyphenyl)ethynyl]pyridazin-3-yl}piperidin-4-ol (Compound No. 62),
4-(2-Fluorobenzyl)-1-{6-[(4-hydroxyphenyl)ethynyl]pyridazin-3-yl}piperidin-4-ol (Compound No. 63),
4-{[6-(4-Hydroxy-4-[(2,5-dichlorobenzyl)piperidin-1-yl)pyridazin-3-yl]ethynyl}phenyl acetate (Compound No. 64),
1-{6-[(4-Hydroxyphenyl)ethynyl]pyridazin-3-yl}-4-(2,5-dichlorobenzyl)piperidin-4-ol (Compound No. 65),
4-[{6-[3-(2-Fluorophenoxyazetidin-1-yl)pyridazin-3-yl}ethynyl]phenyl acetate (Compound No. 66),
4-[{6-[3-(2-Fluorophenoxyazetidin-1-yl)pyridazin-3-yl}ethynyl]phenol (Compound No. 67),
3-(2-{6-[(3S)-3-(2-Fluorophenoxy)azolan-1-yl]-3-pyridazinyl}-1-ethynyl)phenyl acetate (Compound No. 68),
3-(2-{6-[(3S)-3-(2-Fluorophenoxy)azolan-1-yl]-3-pyridyl}-1-ethynyl)phenol (Compound No. 69),
4-[{6-[(3S)-3-(2-Fluorophenoxy)azolan-1-yl]pyridazin-3-yl}ethynyl]phenylacetate (Compound No. 70),
4-[{6-[(3S)-3-(2-Fluorophenoxy)azolan-1-yl]pyridazin-3-yl}ethynyl]phenol (Compound No. 71),
1-[5-(2-Benzo[d][1,3]dioxol-5-yl-1-ethynyl)-2-pyridyl-4-(2-fluorophenox)piperidine (Compound No. 72),
4-(2-Fluorophenoxy)-1-{5-[2-(3-pyridyl)-1-ethynyl]-2-pyridyl}piperidine (Compound No. 73),
4-(2-Fluorophenoxy)-1-(5-{2-[3-(1-oxo)pyridyl]-1-ethynyl}-2-pyridyl)piperidine (Compound No. 74),
4-[{6-[4-(2-Fluorophenoxy)piperidin-1-yl]pyridazin-3-yl}ethynyl]phenyl acetate (Compound No. 75),
4-(2-{6-[4-(2-Fluorophenoxy)piperidino]-3-pyridazinyl}-1-ethynyl)phenol (Compound No. 76),
4-(2-{6-[4-(2-Fluorophenoxy)piperidin-1-yl]pyridazin-3-yl}ethynyl)phenol potassium (Compound No. 77),
3-[4-(2-Fluorophenoxy)piperidin-1-yl]-1-{[4-piperidin-1-ylethoxy]phenylethynyl}-pyridazine (Compound No. 78),
4-{[6-[4-(2-Fluorophenoxy)piperidin-1-yl]pyridazin-3-yl]ethynylphenoxymorpholine (Compound No. 79),
4-{6-[4-(2-Fluorophenoxy)piperidin-1-yl]pyridazin-3-yl}ethynylphenyl-2-furoate (Compound No. 80),
4-(2-{6-[4-Bromo-2-fluorophenoxy)piperidino]-3-pyridazinyl}-1-ethynyl)phenol (Compound No. 81),
2-Fluoro-[4-{6-(4-[2-fluorophenoxy]piperidin-1-yl)pyridazin-3-yl}ethynylphenol (Compound No. 82),
2-Methoxy-4-{6-[4-(2-fluorophenoxy)piperidin-1-yl]pyridazin-3-yl}ethynylphenol (Compound No. 83),
3-[4-(2-Fluorophenoxy)piperidino]-6-[2-(4-trifluoromethylphenyl)-1-ethynyl]pyridazine (Compound No. 84),
3-(2-{6-[4-(2-Fluorophenoxy)piperidin-1-yl]pyridazin-3-yl}ethynyl])phenyl acetate (Compound No. 85),
3-(2-{6-[4-(2-Fluorophenoxy)piperidino]-3-pyridazinyl}-1-ethynyl)phenol (Compound No. 86),
3-[{6-[4-(2-Fluorophenoxy)piperidin-1-yl]pyridazin-3-yl}ethynyl]phenyl pivalate (Compound No. 87),
2-(4-{6-[2-(3-Hydroxyphenyl)-1-ethynyl]-3-pyridazinyl}piperazinoxy)benzonitrile (Compound No. 88),
3-[2-(3-Fluorophenyl)-1-ethynyl]-6-[4-(2-trifluoromethylphenoxy)piperidino]pyridazine (Compound No. 89),
3-(2-{6-[4-(2-Trifluoromethylphenoxy)piperidino]-3-pyridazinyl}-1-ethynyl)phenyl acetate (Compound No. 90),
3-(2-{6-[4-(2-Trifluoromethylphenoxy)piperidino]-3-pyridazinyl}-1-ethynyl)phenol (Compound No. 91),
4-[{6-[4-(2,5-Dichlorophenoxy)piperidin-1-yl]pyridazin-3-yl}ethynyl]phenyl acetate (Compound No. 92),
4-[{6-[4-(2,5-Dichlorophenoxy)piperidin-1-yl]pyridazin-3-yl}ethynyl]phenol (Compound No. 93),
3-[(2,4-Difluoro-3-methoxyphenyl)ethynyl]-6-[4-(2-fluorophenoxy)piperidin-1-yl]Pyridazine (Compound No. 94),
3-[4-(2-Fluorophenoxy)piperidin-1-yl]-6-(1-oxo-pyridin-3-ylethynyl)pyridazine (Compound No. 95),
3-{6-[4-(Pyridin-3-yloxy)lpiperidin-1-yl]pyridazin-3-yl}ethynyl benzamide (Compound No. 96),
1-(2-Fluorophenoxy)-4-{5-[2-(3-methylphenyl)-1-ethynyl]-2-pyrimidinyl}piperazine (Compound No. 97),
3-(2-[4-(2-Fluorophenoxy)piperidino]-5-pyrimidinyl}-1-ethynyl)phenol (Compound No. 98),
4-[(6-{2-[(2-Fluorophenoxy)ethyl]amino}pyridazin-3-yl}ethynyl]phenyl acetate (Compound No. 99),
4-[(6-{2-[(2-Fluorophenoxy)ethyl]amino}pyridazin-3-yl}ethynyl]phenol (Compound No. 100),
4-({6-[4-(2-Fluorophenylamino)piperidin-1-yl]pyridazin-3-yl]pyridazin-3-yl}ethynyl)-phenyl acetate (Compound No. 101),
4-{6-[4-(2-Fluorophenylamino)piperidin-1-yl]pyridazin-3-yl}ethynylphenol (Compound No. 102),
   and pharmaceutically acceptable salts thereof, solvates thereof, stereoisomers thereof, prodrugs thereof, and N-oxides thereof.

TABLE 1

| | |
|---|---|
| | Formula II |
| 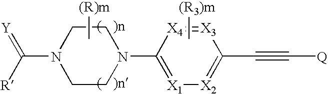 | |

| Com- | | | | | |
|---|---|---|---|---|---|
| pound | | | | | |
| No. | R′ | X₂ | X₃ | X₄ | Q |
| 1 | 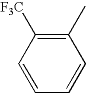 | CH | CH | CH | CH₂OH |
| 2 | 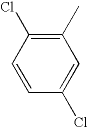 | CH | CH | CH | CH₂OH |
| 3 | 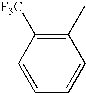 | N | CH | CH | CH₂OH |
| 4 | 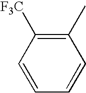 | CH | CH | N | C(CH₃)₂OH |
| 5 | 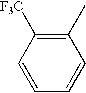 | CH | CH | CH | 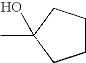 |
| 6 | 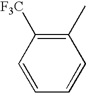 | CH | CH | CH | C(OH)CH₂CH₃ |
| 7 | 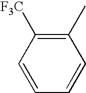 | CH | CH | CH | 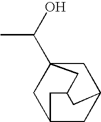 |
| 8 | 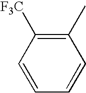 | CH | CH | CH | 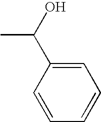 |
| 9 | 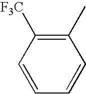 | CH | CH | CH | 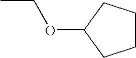 |
| 10 | 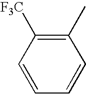 | CH | CH | CH | 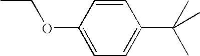 |
| 11 | 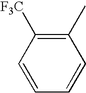 | CH | CH | CH | 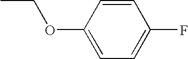 |
| 12 | 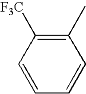 | CH | CH | CH | 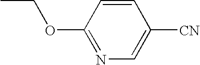 |
| 13 | 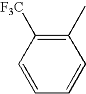 | CH | CH | CH | 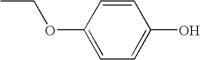 |
| 15 | 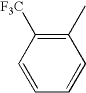 | CH | CH | CH | 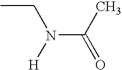 |
| 16 | 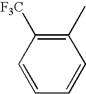 | CH | CH | CH | 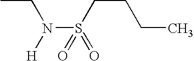 |
| 17 | 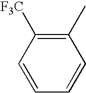 | CH | CH | CH | 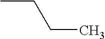 |
| 18 | 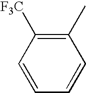 | CH | CH | CH | 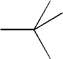 |
| 19 | 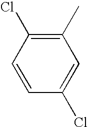 | CH | CH | CH | 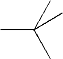 |
| 20 | 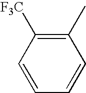 | CH | CH | CH | 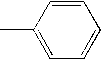 |
| 21 | 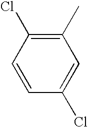 | CH | CH | CH | 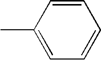 |
| 22 | 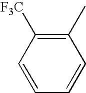 | CH | CH | CH | 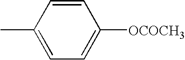 |
| 23 | 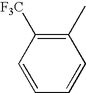 | CH | CH | CH | 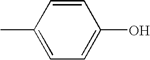 |
| 24 | 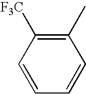 | CH | CH | CH | 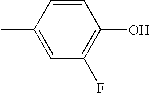 |
| 25 | 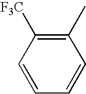 | CH | CH | CH | 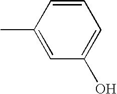 |
| 26 | 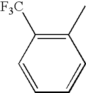 | CH | CH | CH | 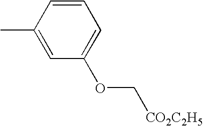 |
| 27 | 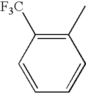 | CH | CH | CH | 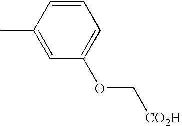 |
| 28 | 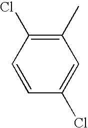 | CH | CH | CH | 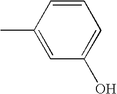 |
| 29 | 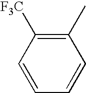 | CH | CH | CH | 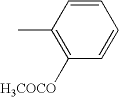 |
| 30 | 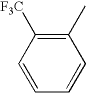 | CH | CH | CH | 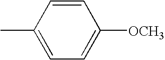 |
| 31 | 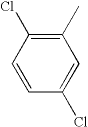 | CH | CH | CH | 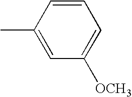 |
| 32 | 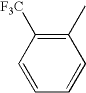 | CH | CH | CH | 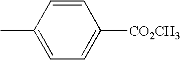 |
| 33 | 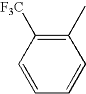 | CH | CH | CH | 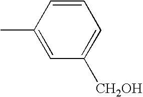 |
| 34 | 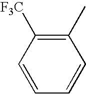 | CH | CH | CH | 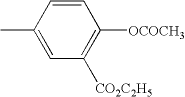 |
| 35 | 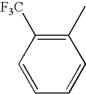 | CH | CH | CH | 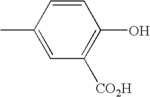 |
| 36 | 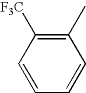 | CH | CH | CH | 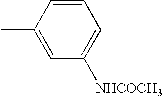 |
| 37 | 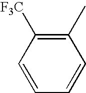 | N | CH | CH | 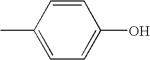 |
| 38 | 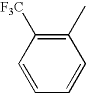 | N | CH | CH | 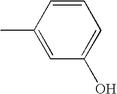 |
| 39 | 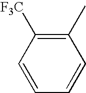 | N | CH | CH | 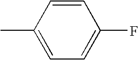 |
| 40 | 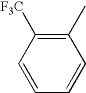 | N | CH | CH | 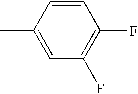 |
| 41 | 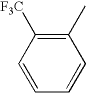 | N | CH | CH | 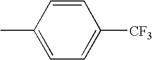 |
| 42 | 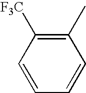 | CH | CH | N | 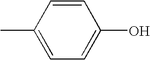 |
| 43 | 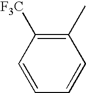 | CH | CH | N | 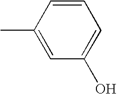 |
| 44 | 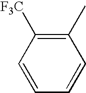 | CH | CH | CH | 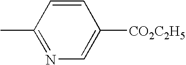 |
| 45 | 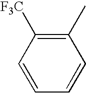 | CH | CH | CH | 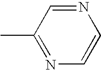 |
| 46 | 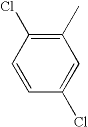 | CH | CH | CH | 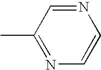 |
| 47 | 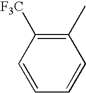 | CH | CH | CH | 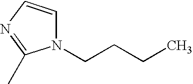 |
| 48 | 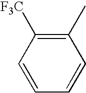 | CH | CH | CH | 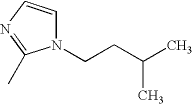 |
| 49 | 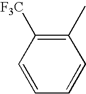 | CH | CH | CH | 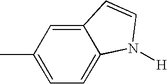 |
| 50 | 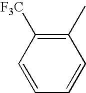 | CH | CH | N | 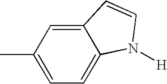 |
| 51 | 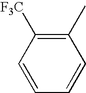 | CH | CH | N | 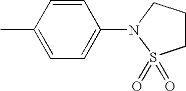 |
| 52 | 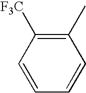 | CH | CH | N | 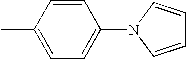 |
| 54 | 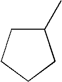 | N | CH | CH | 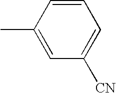 |

n = 1, n′ = 1, R = R₃ = H, Y= O, X₁ = N

TABLE 2

| | |
|---|---|
| | Formula III |
| 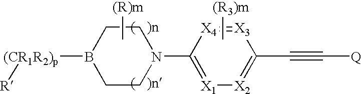 | |

| Com- | | | | | |
|---|---|---|---|---|---|
| pound | | | | | |
| No. | R′ | B | X₂ | X₃ | Q |
| 14* | 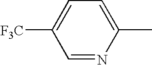 | N | | | 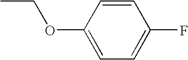 |
| 55 | 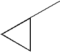 | N | N | CH | 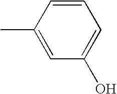 |
| 56 | 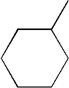 | N | N | CH | 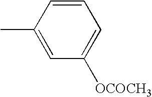 |
| 57 | 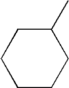 | N | N | CH | 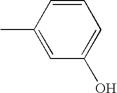 |
| 58 | 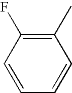 | N | N | CH | 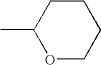 |
| 59 | 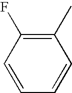 | N | N | CH | 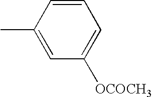 |
| 60 | 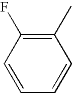 | N | N | CH | 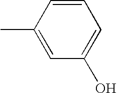 |
| 61 | 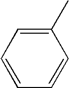 | C(OH) | N | CH | 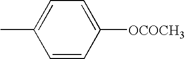 |
| 62 | 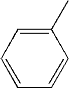 | C(OH) | N | CH | 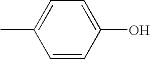 |
| 63 | 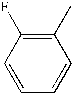 | C(OH) | N | CH | 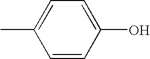 |
| 64 | 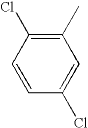 | C(OH) | N | CH | 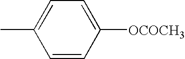 |
| 65 | 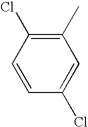 | C(OH) | N | CH | 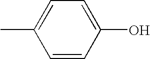 |

X₁ = N, X₄ = CH, n = 1, n′ = 1, R = R₃ = R₁ = R₂ = H, p = 1, *p = 0

TABLE 3

| | |
|---|---|
| | Formula IV |
| 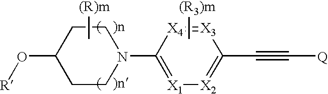 | |

| Com- | | | | | |
|---|---|---|---|---|---|
| pound | | | | | |
| No. | R′ | X₂ | X₃ | X₄ | Q |
| 66** | 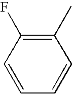 | N | CH | CH | 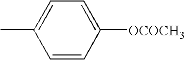 |
| 67** | 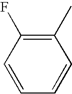 | N | CH | CH | 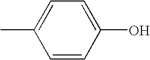 |
| 68* | 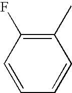 | N | CH | CH | 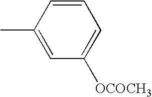 |
| 69* | 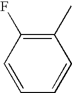 | N | CH | CH | 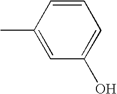 |
| 70* | 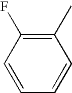 | N | CH | CH | 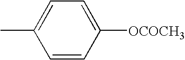 |
| 71* | 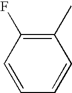 | N | CH | CH | 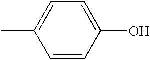 |
| 72 | 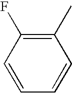 | CH | CH | CH | 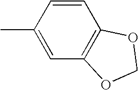 |
| 73 | 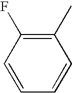 | CH | CH | CH | 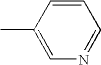 |
| 74 | 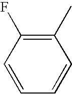 | CH | CH | CH | 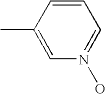 |
| 75 | 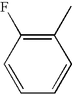 | N | CH | CH | 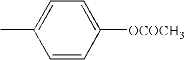 |
| 76 | 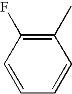 | N | CH | CH | 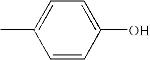 |
| 77 | 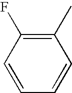 | N | CH | CH | 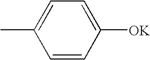 |
| 78 | 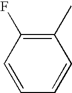 | N | CH | CH | 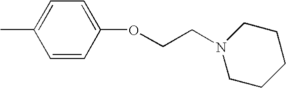 |
| 79 | 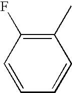 | N | CH | CH | 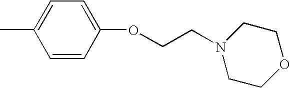 |
| 80 | 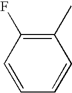 | N | CH | CH | 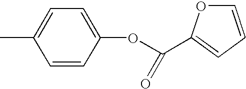 |
| 81 | 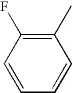 | N | CH | CH | 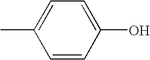 |
| 82 | 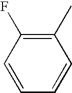 | N | CH | CH | 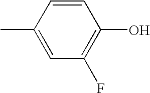 |
| 83 | 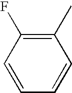 | N | CH | CH | 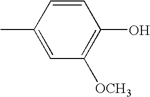 |
| 84 | 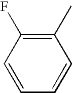 | N | CH | CH | 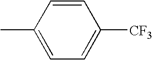 |
| 85 | 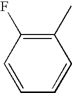 | N | CH | CH | 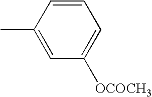 |
| 86 | 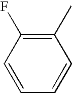 | N | CH | CH | 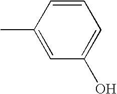 |
| 87 | 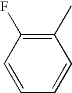 | N | CH | CH | 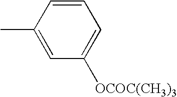 |
| 88 | 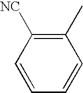 | N | CH | CH | 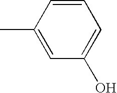 |
| 89 | 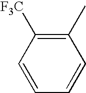 | N | CH | CH | 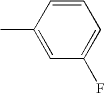 |
| 90 | 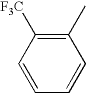 | N | CH | CH | 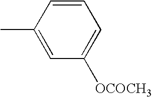 |
| 91 | 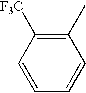 | N | CH | CH | 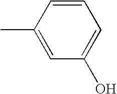 |
| 92 | 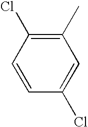 | N | CH | CH | 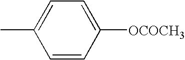 |
| 93 | 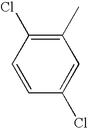 | N | CH | CH | 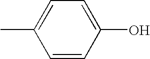 |
| 94 | 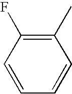 | N | CH | CH | 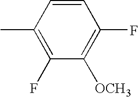 |
| 95 | 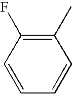 | N | CH | CH | 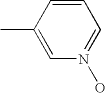 |
| 96 | 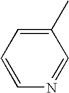 | N | CH | CH | 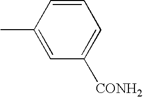 |
| 97 | 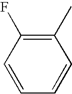 | CH | CH | N | 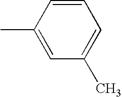 |
| 98 | 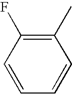 | CH | CH | N | 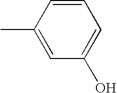 |

X₁ = N, n = 1, n′ = 1, R = R₃ = H, (*n′ = 0, **n = n′ = 0)

TABLE 4

| | |
|---|---|
| | Formula V |
| 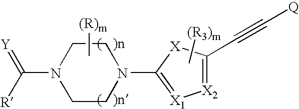 | |

| Com- | | | | |
|---|---|---|---|---|
| pound | | | | |
| No. | R′ | X | X₂ | Q |
| 53 | 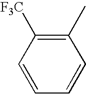 | S | CH | 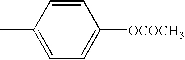 |

X₁ = N, n = n′ = 1, R = R₃ = H, (*n′ = 0, **n = n′ = 0), Y = O

TABLE 5

| | |
|---|---|
| | Formula VI |
| 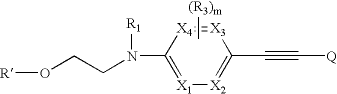 | |

| Com- | | | | |
|---|---|---|---|---|
| pound | | | | |
| No. | R′ | X₃ | X₄ | Q |
| 99 | 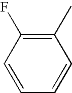 | CH | CH | 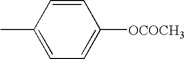 |
| 100 | 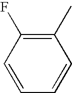 | CH | CH | 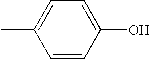 |

X₁ = X₂ = N, R₁ = R₃ = H

TABLE 6

| | |
|---|---|
| | Formula VII |
| 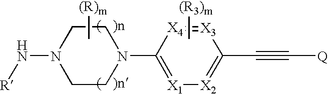 | |

| Com- | | | | |
|---|---|---|---|---|
| pound | | | | |
| No. | R′ | X₃ | X₄ | Q |
| 101 | 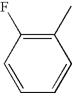 | CH | CH | 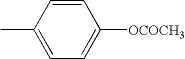 |
| 102 | 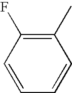 | CH | CH | 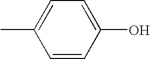 |

X₁ = X₂ = N, n = 1, n′ = 1, R = R₃ = H

In another aspect, provided herein is a pharmaceutical composition comprising a therapeutically effective amount of one or more compounds of any of Formula I-VIII, and optionally one or more pharmaceutically acceptable excipients (e.g., carriers or diluents).

In another aspect, provided herein is a method for preventing, ameliorating or treating a disease, disorder or syndrome modulated by SCD in a subject comprising administering to the subject in need thereof a therapeutically effective amount of one or more compounds of any of Formula I-VIII, or a pharmaceutical composition as described herein.

In another aspect, provided herein is a method for preventing, ameliorating or treating a disease, disorder or syndrome modulated by SCD1 in a subject comprising administering to the subject in need thereof a therapeutically effective amount of one or more compounds of any of Formula I-VIII, or a pharmaceutical composition as described herein.

The diseases, disorders, and syndromes can be selected from obesity (for example, obesity resulting from genetics, diet, food intake volume, a metabolic disorder, a hypothalmic disorder, age, abnormal adipose mass distribution, abnormal adipose compartment distribution, compulsive eating disorders, motivational disorders, which include the desire to consume sugars, carbohydrates, alcohols or drugs or any ingredient with hedonic value, reduced activity or combination thereof); overweight conditions; anorexia; bulimia; cachexia; dysregulated appetite; obesity related diseases, disorders, and symptoms; diabetes (including Type I and Type II diabetes); diabetic complications; glucose tolerance; hyperinsulinemia; insulin sensitivity or resistance; hepatic steatosis; increased abdominal girth; metabolic syndrome; cardiovascular diseases (including, for example, atherosclerosis, dyslipidemia, elevated blood pressure, microalbuminemia, hyperuricaemia, hypercholesterolemia, hyperlipidemias, atherosclerosis, hypertriglyceridemias, arteriosclerosis or combination thereof); osteoarthritis; dermatological diseases; sleep disorders (including, for example, disturbances of circadian rhythm, dysomnia, insomnia, sleep apnea, narcolepsy or combination thereof); cholelithiasis; hepatomegaly; steatosis; syndrome X; abnormal alanine aminotransferase levels; polycystic ovarian disease; inflammation; non-alcoholic fatty liver disease; skin disorder; respiratory diseases or disorders (including, for example, sinusitis, asthma, bronchitis or combination thereof); pancreatitis; rheumatoid arthritis; cystic fibrosis; pre-menstrual syndrome; cancer; neoplasia; malignancy; metastases; tumours (benign or malignant); hepatomas; neurological diseases; psychiatric disorders; multiple sclerosis; viral diseases/infections or any combination thereof; diseases or conditions related to serum levels of triglyceride, LDL, HDL, VLDL, or total chlolesterol.

In one embodiment, there is provided a method for preventing, ameliorating or treating a disease or condition selected from obesity or related diseases or conditions; diabetes (including Type I and Type II diabetes); diabetic complications; glucose tolerance; hyperinsulinemia; insulin sensitivity or resistance; metabolic syndromes; cardiovascular diseases (including, for example, atherosclerosis, hypertension, lipidemia, dyslipidemia, elevated blood pressure, microalbuminemia, hyperuricaemia, hypercholesterolemia, hyperlipidemias, hypertriglyceridemias, arteriosclerosis or combination thereof); respiratory diseases or disorders (including, for example, sinusitis, asthma, bronchitis or combination thereof); diseases or conditions related to serum levels of triglyceride, LDL, HDL, VLDL or total chlolesterol.

In another embodiment, there is provided a method for preventing, ameliorating or treating a disease or condition selected from obesity or related diseases or conditions, Type II diabetes, atherosclerosis, hypertension, lipidemia, dyslipidemia, microalbuminemia, hyperuricaemia, hypercholesterolemia, hyperlipidemias, hypertriglyceridemias, or a combination thereof;

In another embodiment, provided herein is a method for preventing, ameliorating or treating a disease or condition related to serum levels of triglyceride, LDL, HDL, VLDL, total chlolesterol or a combination thereof.

In yet another embodiment there is provided a method for preventing, ameliorating or treating a disease or condition selected from obesity or a complication thereof, type II diabetes or a complication thereof; cardiovascular diseases or a complication thereof, or a combination of these.

In another aspect, provided herein is a method for treating a disease or disorder described herein comprising administering concurrently or sequentially one or more compounds described herein with one or more active ingredients for the disease or disorder known to those skilled in the art.

The combination therapy can include one or more of the following embodiments. For example, the one or more active ingredients can be selected from antidiabetic agents including, for example, PPARα, PPARγ and/or PPARδ agonists or antagonists, sulfonylurea drugs, non-sulfonylurea secretogogues, α-glucosidase inhibitors, insulin sensitizers, hepatic glucose output lowering compounds, insulin and insulin derivatives or a combination thereof.

In another embodiment, the one or more active ingredients are selected from antiobesity drugs including, for example, β-3 agonists, CB (CB1 and/or CB2) receptor modulators, neuropeptide Y5 inhibitors, ciliary neurotropic factor and derivatives, appetite suppressants or a combination thereof.

In another embodiment, the one or more active ingredients are selected from HMG CoA reductase inhibitors, CETP inhibitors, lipid lowering drugs, fatty acid lowering compounds, ACAT inhibitors, bile acid sequestrants, bile acid reuptake inhibitors, microsomal triglycerides transport inhibitors, fibric acid derivatives, guggle lipids or a combination thereof.

In yet another embodiment, the one or more active ingredients are selected from antihypertensive drugs including, for example, β-blockers, ACE inhibitors, calcium channel blockers, diuretics, renine inhibitors, AT-1 receptor antagonists, Endothelin receptor antagonists or a combination thereof.

In yet another aspect, provided herein are processes for preparing compounds of the present invention.

### DETAILED DESCRIPTION OF THE INVENTION

The following definitions apply to the terms as used herein.

The term “alkyl” unless otherwise specified refers to an optionally substituted straight or branched saturated hydrocarbon chain having 1 to 12 carbon atoms, which is attached to the rest of the molecule by a single bond, e.g., methyl, ethyl, n-propyl, 1-methylethyl (isopropyl), n-butyl, n-pentyl, and 1,1-dimethylethyl (t-butyl). The term “alkenyl” unless otherwise specified refers to an optionally substituted aliphatic hydrocarbon group containing at least one double bond and which may be a straight or branched chain having 2 to about 10 carbon atoms, with cis or trans; E or Z stereochemistry e.g., ethenyl, 1-propenyl, 2-propenyl (allyl), iso-propenyl, 2-methyl-1-propenyl, 1-butenyl, and 2-butenyl. The term “alkynyl” unless otherwise specified refers to an optionally substituted straight or branched chain hydrocarbyl radical having at least one carbon-carbon triple bond, and having 2 to about 12 carbon atoms, e.g., ethynyl, propynyl, and butynyl.

The term “alkoxy” unless otherwise specified refers to an alkyl group attached via an oxygen linkage to the rest of the molecule. Representative examples of such groups are —OCH₃ and —OC₂H₅.

The term “cycloalkyl” unless otherwise specified refers to an optionally substituted non-aromatic mono or multicyclic ring system of 3 to about 12 carbon atoms, which may optionally contain one or more olefinic bonds unless constrained by the definition, such as cyclopropyl, cyclobutyl, cyclopentyl, and cyclohexyl. It also includes the cyclic ring system fused with an aryl ring, spiro systems. Examples of multicyclic cycloalkyl groups include, but are not limited to, perhydronapththyl, adamantyl and norbornyl groups, bridged cyclic groups and spirobicyclic groups, e.g., spiro (4,4) non-2-yl.

The term “cycloalkylalkyl” unless otherwise specified refers to optionally substituted cyclic ring-containing radical having 3 to about 8 carbon atoms directly attached to an alkyl group. The cycloalkylalkyl group may be attached to the main structure at any carbon atom in the alkyl group that results in the creation of a stable structure. Non-limiting examples of such groups include cyclopropylmethyl, cyclobutylethyl, and cyclopentylethyl.

The term “cycloalkenyl” refers to optionally substituted cyclic ring-containing radical having 3 to about 12 carbon atoms with at least one carbon-carbon double bond, such as cyclopropenyl, cyclobutenyl, and cyclopentenyl.

The term “cycloalkenylalkyl” unless otherwise specified refers to optionally substituted cycloalkenyl ring directly attached to an alkyl group.

The term “aryl” unless otherwise specified refers to an optionally substituted carbocyclic aromatic radical having 6 to 14 carbon atoms, wherein the ring is mono-, bi-, or tricyclic, such as, but not limited to, phenyl, naphthyl, tetrahydronapthyl, indanyl, and biphenyl.

The term “arylalkyl” unless otherwise specified refers to an optionally substituted aryl group as defined above directly bonded to an alkyl group as defined above, e.g., —CH₂C₆H₅ and —C₂H₅C₆H₅.

The term “heterocyclic ring” or “heterocyclyl” unless otherwise specified refers optionally substituted non-aromatic 3 to 15 membered ring radical which consists of carbon atoms and from one to five heteroatoms selected from nitrogen, phosphorus, oxygen and sulfur. The heterocyclic ring radical may be a mono-, bi- or tricyclic ring system, which may include fused, bridged or spiro ring systems, and the nitrogen, phosphorus, carbon, oxygen or sulfur atoms in the heterocyclic ring radical may be optionally oxidized to various oxidation states. In addition, the nitrogen atom may be optionally quaternized; also, unless otherwise constrained by the definition the heterocyclic ring or heterocyclyl may optionally contain one or more olefinic bond(s). Examples of such heterocyclic ring radicals include, but are not limited to, azepinyl, azetidinyl, acridinyl, benzodioxolyl, benzodioxanyl, benzofurnyl, carbazolyl, cinnolinyl, dioxolanyl, indolizinyl, thienyl, naphthyridinyl, perhydroazepinyl, phenazinyl, phenothiazinyl, phenoxazinyl, indolyl, phthalazinyl, pyridyl, pteridinyl, purinyl, quinazolinyl, quinoxalinyl, quinolinyl, isoquinolinyl, tetrazoyl, imidazolyl, tetrahydroisouinolyl, piperidinyl, piperazinyl, 2-oxopiperazinyl, 2-oxopiperidinyl, 2-oxopyrrolidinyl, 2-oxoazepinyl, pyrrolyl, 4-piperidonyl, pyrrolidinyl, pyrazinyl, pyrimidinyl, pyridazinyl, oxazolyl, oxazolinyl, oxasolidinyl, triazolyl, indanyl, isoxazolyl, isoxasolidinyl, morpholinyl, thiazolyl, thiazolinyl, thiazolidinyl, isothiazolyl, quinuclidinyl, isothiazolidinyl, isoindolyl, indolinyl, isoindolinyl, octahydroindolyl, octahydroisoindolyl, quinolyl, isoquinolyl, decahydroisoquinolyl, benzimidazolyl, thiadiazolyl, benzopyranyl, benzothiazolyl, benzooxazolyl, furyl, tetrahydrofuryl, tetrahydropyranyl, benzothienyl, thiamorpholinyl, thiamorpholinyl sulfoxide, thiamorpholinyl sulfone, dioxaphospholanyl, oxadiazolyl, chromanyl, and isochromanyl. The heterocyclic ring radical may be attached to the main structure at any heteroatom or carbon atom that results in the creation of a stable structure.

The term “heteroaryl” unless otherwise specified refers to optionally substituted 5 to 14 membered aromatic heterocyclic ring radical with one or more heteroatom(s) independently selected from N, O or S. The heteroaryl may be a mono-, bi- or tricyclic ring system. The heteroaryl ring radical may be attached to the main structure at any heteroatom or carbon atom that results in the creation of a stable structure. Examples of such heteroaryl ring radicals include, but are not limited to, oxazolyl, imidazolyl, pyrrolyl, furanyl, triazinyl, pyridinyl, pyrimidinyl, pyrazinyl, benzofuranyl, indolyl, benzothiazolyl, benzoxazolyl, carbazolyl, quinazonyl and the like.

The term “heteroarylalkyl” unless otherwise specified refers to optionally substituted heteroaryl ring radical directly bonded to an alkyl group. The heteroarylalkyl radical may be attached to the main structure at any carbon atom in the alkyl group that results in the creation of a stable structure, wherein the heteroaryl and alkyl are the same as defined earlier.

The term “heterocyclylalkyl” unless otherwise specified refers to optionally substituted heterocyclic ring radical directly bonded to an alkyl group. The heterocyclylalkyl radical may be attached to the main structure at any carbon atom in the alkyl group that results in the creation of a stable structure wherein the heterocyclyl and alkyl are the same as defined earlier.

Unless otherwise specified, the term “substituted” as used herein refers to substitution with any one or any combination of the following substituents: hydroxy, halogen, carboxyl, cyano, nitro, oxo (═O), thio (═S), substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkylalkyl, substituted or unsubstituted cycloalkenylalkyl, substituted or unsubstituted cycloalkenyl, substituted or unsubstituted amino, substituted or unsubstituted heteroaryl, substituted or unsubstituted heterocyclylalkyl ring, substituted or unsubstituted heteroarylalkyl, substituted or unsubstituted heterocyclic ring, substituted or unsubstituted guanidine, —COOR^{x}, —C(O)R^{x}, —C(S)R^{x}, —C(O)NR^{x}R^{y}, —C(O)ONR^{x}R^{y}, —NR^{x}CONR^{y}R^{z}, —N(R^{x})SOR^{y}, —N(R^{x})SO₂R^{y}, —(═N—N(R^{x})R^{y}), —NR^{x}C(O)OR^{y}, —NR^{x}R^{y}, —NR^{x}C(O)R^{y}, —NR^{x}C(S)R^{y}, —NR^{x}C(S)NR^{y}R^{z}, —SONR^{x}R^{y}, —SO₂NR^{x}R^{y}, —OR^{x}, —OR^{x}C(O)NR^{y}R^{z}, —OR^{x}C(O)OR^{y}, —OC(O)R^{x}, —OC(O)NR^{x}R^{y}, —OC(O)NR^{y}R^{z}, —R^{x}NR^{y}C(O)R^{z}, —R^{x}OR^{y}, —R^{x}C(O)OR^{y}, —R^{x}C(O)NR^{y}R^{z}, —R^{x}C(O)R^{y}, —R^{x}OC(O)R^{y}, —SR^{x}, —SOR^{x}, —SO₂R^{x}, and —ONO₂, wherein R^{x}, R^{y} and R^{z} are independently selected from hydrogen, substituted or unsubstituted alkyl, substituted or unsubstituted alkylene, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted heterocyclic ring, substituted or unsubstituted heterocyclylalkyl, or substituted or unsubstituted amino. The substituents in the aforementioned “substituted” groups cannot be further substituted. For example, when the substituent on “substituted alkyl” is “substituted aryl”, the substituent on “substituted aryl” cannot be “substituted alkenyl”.

The term “protecting group” or “P” refers to a substituent that is employed to block or protect a particular functionality while other functional groups on the compound may remain reactive. For example, an “amino-protecting group” is a substituent attached to an amino group that blocks or protects the amino functionality in the compound. Suitable amino-protecting groups include, but are not limited to, acetyl, trifluoroacetyl, t-butoxycarbonyl (BOC), benzyloxycarbonyl (CBz) and 9-fluorenylmethylenoxycarbonyl (Fmoc). Similarly, a “hydroxy-protecting group” refers to a substituent of a hydroxy group that blocks or protects the hydroxy functionality. Suitable hydroxy-protecting groups include, but are not limited to, acetyl, benzyl, tetrahydropyranyl and silyl. A “carboxy-protecting group” refers to a substituent of the carboxy group that blocks or protects the carboxy functionality. Suitable carboxy-protecting groups include, but are not limited to, —CH₂CH₂SO₂Ph, cyanoethyl, 2-(trimethylsilyl)ethyl, 2-(trimethyl silyl)ethoxymethyl, 2-(p-toluenesulfonyl)ethyl, 2-(p-nitrophenylsulfenyl)ethyl, 2-(diphenyl phosphino)-ethyl, and nitroethyl. For a general description of protecting groups and their use, see, T. W. Greene, Protective Groups in Organic Synthesis, John Wiley & Sons, New York, 1991.

The term “prodrug” refers to a compound that is transformed in vivo to yield a compound of formula (I) or a pharmaceutically acceptable salt, hydrate or solvate of the compound. The transformation may occur by various mechanisms, such as through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

The term “treating” or “treatment” of a state, disorder or condition includes:
(1) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition;
(2) inhibiting the state, disorder or condition, i.e., arresting or reducing the development of the disease or at least one clinical or subclinical symptom thereof; or
(3) relieving the disease, i.e., causing regression of the state, disorder or condition or at least one of its clinical or subclinical symptoms.

The benefit to a subject to be treated is either statistically significant or at least perceptible to the subject or to the physician.

The term “subject” includes mammals (especially humans) and other animals, such as domestic animals (e.g., household pets including cats and dogs) and non-domestic animals (such as wildlife).

A “therapeutically effective amount” means the amount of a compound that, when administered to a subject for treating a state, disorder or condition, is sufficient to effect such treatment. The “therapeutically effective amount” will vary depending on the compound, the disease and its severity and the age, weight, physical condition and responsiveness of the subject to be treated.

Pharmaceutically acceptable salts forming part of this invention include salts derived from inorganic bases (such as Li, Na, K, Ca, Mg, Fe, Cu, Zn, and Mn), salts of organic bases (such as N,N′-diacetylethylenediamine, glucamine, triethylamine, choline, hydroxide, dicyclohexylamine, metformin, benzylamine, trialkylamine, and thiamine), salts of chiral bases (such as alkylphenylamine, glycinol, and phenyl glycinol), salts of natural amino acids (such as glycine, alanine, valine, leucine, isoleucine, norleucine, tyrosine, cystine, cysteine, methionine, proline, hydroxy proline, histidine, ornithine, lysine, arginine, and serine), salts of non-natural amino acids (such as D-isomers or substituted amino acids), salts of guanidine, salts of substituted guanidine (wherein the substituents are selected from nitro, amino, alkyl, alkenyl, or alkynyl), ammonium salts, substituted ammonium salts, and aluminum salts. Other pharmaceutically acceptable salts include acid addition salts (where appropriate) such as sulphates, nitrates, phosphates, perchlorates, borates, hydrohalides, acetates (such as trifluoroacetate), tartrates, maleates, citrates, fumarates, succinates, palmoates, methanesulphonates, benzoates, salicylates, benzenesulfonates, ascorbates, glycerophosphates, and ketoglutarates. Yet other pharmaceutically acceptable salts include, but are not limited to, quaternary ammonium salts of the compounds of invention with alkyl halides or alkyl sulphates (such as MeI or (Me)₂SO₄).

Pharmaceutically acceptable solvates include hydrates and other solvents of crystallization (such as alcohols). The compounds of the present invention may form solvates with low molecular weight solvents by methods known in the art.

Compounds described herein can comprise one or more asymmetric carbon atoms and thus can occur as racemic mixtures, enantiomers and diastereomers. These compounds can also exist as conformers/rotamers. All such isomeric forms are expressly included in the present invention. Although the specific compounds exemplified in this application may be depicted in a particular stereochemical configuration, compounds having either the opposite stereochemistry at any given chiral centre are envisioned as a part thereof.

### Pharmaceutical Compositions

The pharmaceutical composition of the present invention comprises one or more compounds described herein and one or more pharmaceutically acceptable excipients, carriers, diluents or mixture thereof. The compounds described herein may be associated with one or more pharmaceutically acceptable excipients, carriers, diluents or mixture thereof in the form of capsule, sachet, paper or other container.

Examples of suitable carriers include, but are not limited to, water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethyl cellulose and polyvinylpyrrolidone.

The carrier or diluent may include a sustained release material, such as glyceryl monostearate or glyceryl distearate, alone or mixed with a wax.

The pharmaceutical composition may also include one or more pharmaceutically acceptable auxiliary agents, wetting agents, emulsifying agents, suspending agents, preserving agents, salts for influencing oxmetic pressure, buffers, sweetening agents, flavoring agents, colorants, or any combination of the foregoing. The pharmaceutical composition of the invention may be formulated so as to provide quick, sustained, or delayed release of the active ingredient after administration to the subject by employing methods known in the art.

The pharmaceutical compositions of the present invention may be prepared by conventional techniques, e.g., as described in Remington: The Science and Practice of Pharmacy, 20^{th} Ed., 2003 (Lippincott Williams & Wilkins). For example, the active compound is mixed with a carrier, or diluted by a carrier, or enclosed within a carrier, which may be in the form of an ampoule, capsule, sachet, paper, or other container. When the carrier serves as a diluent, it may be a solid, semi-solid, or liquid material that acts as a vehicle, excipient, or medium for the active compound. The active compound is adsorbed on a granular solid container, for example, in a sachet.

The pharmaceutical compositions may be in conventional forms, for example, capsules, tablets, aerosols, solutions, suspensions or products for topical application.

The route of administration may be any route which effectively transports the active compound of the invention to the appropriate or desired site of action. Suitable routes of administration include, but are not limited to, oral, nasal, pulmonary, buccal, subdermal, intradermal, transdermal, parenteral, rectal, depot, subcutaneous, intravenous, intraurethral, intramuscular, intranasal, ophthalmic (such as with an ophthalmic solution) or topical (such as with a topical ointment). The oral route is preferred.

Solid oral formulations include, but are not limited to, tablets, capsules (soft or hard gelatin), dragees (containing the active ingredient in powder or pellet form), troches and lozenges. Tablets, dragees, or capsules having talc and/or a carbohydrate carrier or binder or the like are particularly suitable for oral application. Preferable carriers for tablets, dragees, or capsules include lactose, cornstarch, and/or potato starch. A syrup or elixir is used in cases where a sweetened vehicle is employed.

A typical tablet that may be prepared by conventional tabletting techniques may contain: (1) Core: Active compound (as free compound or salt thereof), 250 mg colloidal silicon dioxide (Aerosil®), 1.5 mg microcrystalline cellulose (Avicel®), 70 mg modified cellulose gum (Ac-Di-Sol®), and 7.5 mg magnesium stearate; (2) Coating: HPMC, approx. 9 mg Mywacett 9-40 T and approx. 0.9 mg acylated monoglyceride

Liquid formulations include, but are not limited to, syrups, emulsions, soft gelatin and sterile injectable liquids, such as aqueous or non-aqueous liquid suspensions or solutions.

For parenteral application, particularly suitable are injectable solutions or suspensions, preferably aqueous solutions with the active compound dissolved in polyhydroxylated castor oil.

Suitable doses of the compounds for use in treating the diseases and disorders described herein can be determined by those skilled in the relevant art. Therapeutic doses are generally identified through a dose ranging study in humans based on preliminary evidence derived from the animal studies. Doses must be sufficient to result in a desired therapeutic benefit without causing unwanted side effects. For example, the daily dosage of the SCD1 inhibitor can range from about 0.5 to about 3 mg/kg. Mode of administration, dosage forms, suitable pharmaceutical excipients, diluents or carriers can also be well used and adjusted by those skilled in the art. All changes and modifications are envisioned within the scope of the present invention.

### Methods of Treatments and Combination Therapy

The present invention further provides a method of treating a disease, condition or disorder modulated by a stearoyl CoA desaturase, especially those modulated by SCD1, in a subject by administering to the subject in need thereof a therapeutically effective amount of a compound or a pharmaceutical composition described herein.

Diseases, conditions, and disorders that are modulated by a stearoyl CoA desaturase, include, but are not limited to, diabetes, diabetes related syndromes, disorders or diseases, obesity, obesity related diseases, conditions, and disorders, cardiovascular diseases (such as atherosclerosis), hepatic steatosis and other metabolic syndromes, metabolism related syndromes, disorders and diseases, and non-alcoholic fatty liver disease.

SCD, particularly human SCD, can be regulated to treat obesity. Obesity and overweight are defined as an excess of body fat relative to lean body mass. An increase in caloric intake or a decrease in energy expenditure or both can bring about this imbalance leading to surplus energy being stored as fat. In contrast, anorexia and cachexia are characterized by an imbalance in energy intake versus energy expenditure leading to a negative energy balance and weight loss. Agents that either increase energy expenditure and/or decrease energy intake, absorption or storage would be useful for treating obesity, overweight, and associated comorbidities. Agents that increase energy intake and/or decrease energy expenditure or increase the amount of lean tissue would be useful for treating cachexia, anorexia, and wasting disorders. An SCD gene, translated proteins and agents which modulate the gene or portions of the gene or its products are useful for treating obesity, overweight, anorexia, cachexia, wasting disorders, appetite suppression, appetite enhancement, increases or decreases in satiety, modulation of body weight, and/or other eating disorders such as bulimia. Accordingly, diseases, conditions, and disorders that are modulated by a stearoyl CoA desaturase, include, but are not limited to, obesity, overweight, anorexia, cachexia, wasting disorders, appetite suppression, appetite enhancement, and/or other eating disorders such as bulimia. Furthermore, the compounds of the present invention increase or decrease satiety and modulate body weight.

Obesity related syndromes, disorders and diseases include, but are not limited to, obesity as a result of (i) genetics, (ii) diet, (iii) food intake volume, (iv) a metabolic disorder, (v) a hypothalmic disorder, (vi) age, (vii) abnormal adipose mass distribution, (viii) abnormal adipose compartment distribution, (ix) compulsive eating disorders, and (x) motivational disorders which include the desire to consume sugars, carbohydrates, alcohols or drugs or any ingredient with hedonic value. Symptoms associated with obesity related syndromes, disorders, and diseases include, but are not limited to, reduced activity. Obesity also increases the likelihood of sleep apnea, gallstones, osteoporosis and ceratin cancers.

Diabetes related syndromes, disorders and diseases include, but are not limited to, glucose dysregulation, insulin resistance, glucose intolerance, hyperinsulinemia, dyslipidemia, hypertension, obesity, and hyperglycemia.

Cardiovascular diseases include, but are not limited to, (i) coronary artery disease, (ii) atherosclerosis, (iii) heart disease, (iv) hypercholesterolemia, (v) hypertriglyceridemia, (vi) hypertriglyceridemia secondary to another disorder or disease (such as hyperlipoproteinemias), (vii) hyperlipidemia, (viii) disorders of serum levels of triglycerides, VLDL, HDL, and LDL, (ix) cholesterol disorders, (x) cerebrovascular disease (including but not limited to, stroke, ischemic stroke and transient ischemic attack (TIA)), (xi) peripheral vascular disease, and (xii) ischemic retinopathy.

Metabolism related syndromes, disorders or diseases include, but are not limited to, (i) metabolic syndrome, (ii) dyslipidemia, (iii) elevated blood pressure, (iv) insulin sensitivity or resistance, (v) Type II diabetes, (vi) Type I diabetes, (vii) diabetic complications, (viii) increased abdominal girth, (ix) glucose tolerance, (x) microalbuminemia, (xi) hyperuricaemia, (xii) hyperinsulinemia, (xiii) hypercholesterolemia, (xiv) hyperlipidemias, (xv) atherosclerosis, (xvi) hypertriglyceridemias, (xvii) arteriosclerosis and other cardiovascular diseases, (xviii) osteoarthritis, (xix) dermatological diseases, (xx) sleep disorders (e.g., disturbances of circadian rhythm, dysomnia, insomnia, sleep apnea and narcolepsy), (xxi) cholelithiasis, (xxii) hepatomegaly, (xxiiii) steatosis, (xxiv) syndrome X, (xxv) abnormal alanine aminotransferase levels, (xxvi) polycystic ovarian disease, and (xxvii) inflammation.

Non-alcoholic fatty liver disease can manifest as hepatic steatosis (or fatty liver) and can progress to hepatitis, drug-induced hepatitis, hepatoma, fibrosis, hepatic cirrhosis, liver failure, non-alcoholic steatohepatitis, non-alcoholic hepatitis, acute fatty liver, and fatty liver of pregnancy.

Other disorders or diseases mediated by SCD include, but are not limited to, skin disorder, inflammation, respiratory diseases or disorders (e.g., sinusitis, asthma, and bronchitis), pancreatitis, osteoarthritis, rheumatoid arthritis, cystic fibrosis, pre-menstrual syndrome, cancer, neoplasia, malignancy, metastases, tumours (benign or malignant), carcinogenesis, hepatomas, neurological diseases, psychiatric disorders, multiple sclerosis, and viral diseases and infections.

In a preferred embodiment, compounds of the invention will, in a subject, increase HDL levels and/or decrease triglyceride levels and/or decrease LDL or non-HDL-cholesterol levels. In another embodiment, compounds of the invention will, in a subject, increase body lean mass and decrease obesity. In another embodiment, compounds of the invention will, in a subject, decrease hepatitic steatosis.

Methods described herein can also include one or more of the following embodiments. For example, in one embodiment, the diseases, disorders, and syndromes are selected, but are not limited to, obesity, for example, obesity resulting from genetics, diet, food intake volume, a metabolic disorder, a hypothalmic disorder, age, abnormal adipose mass distribution, abnormal adipose compartment distribution, compulsive eating disorders, motivational disorders, which include the desire to consume sugars, carbohydrates, alcohols or drugs or any ingredient with hedonic value, reduced activity or combination thereof; overweight conditions; anorexia; bulimia; cachexia; dysregulated appetite; or obesity related diseases, disorders, and symptoms; diabetes (including Type I and Type II diabetes); diabetic complications; glucose tolerance; hyperinsulinemia; insulin sensitivity or resistance; hepatic steatosis; increased abdominal girth; metabolic syndrome; cardiovascular diseases including, for example, atherosclerosis, dyslipidemia, elevated blood pressure, microalbuminemia, hyperuricaemia, hypercholesterolemia, hyperlipidemias, atherosclerosis, hypertriglyceridemias, arteriosclerosis or combination thereof; osteoarthritis; dermatological diseases; sleep disorders including, for example, disturbances of circadian rhythm, dysomnia, insomnia, sleep apnea, narcolepsy or combination thereof; cholelithiasis; hepatomegaly; steatosis; syndrome X; abnormal alanine aminotransferase levels; polycystic ovarian disease; inflammation; non-alcoholic fatty liver disease; skin disorder; respiratory diseases or disorders including, for example, sinusitis, asthma, bronchitis or combination thereof; pancreatitis; rheumatoid arthritis; cystic fibrosis; pre-menstrual syndrome; cancer; neoplasia; malignancy; metastases; tumours (benign or malignant); hepatomas; neurological diseases; psychiatric disorders; multiple sclerosis; viral diseases/infections or any combination these diseases, disorders, conditions and/or syndromes thereof; the disease or condition related to serum levels of triglyceride, LDL, HDL, VLDL, total chlolesterol.

In another embodiment, there is provided a method for preventing, ameliorating or treating a disease or condition selected from obesity or related diseases, conditions; diabetes (including Type I and Type II diabetes); diabetic complications; glucose tolerance; hyperinsulinemia; insulin sensitivity or resistance; metabolic syndromes; cardiovascular diseases including, for example, atherosclerosis, lipidemia, dyslipidemia, elevated blood pressure, microalbuminemia, hyperuricaemia, hypercholesterolemia, hyperlipidemias, hypertriglyceridemias, arteriosclerosis or combination thereof; respiratory diseases or disorders including, for example, sinusitis, asthma, bronchitis or combination thereof; or any combination these diseases, disorders, conditions and/or syndromes thereof; the disease or condition related to serum levels of triglyceride, LDL, HDL, VLDL, total chlolesterol.

In another embodiment, there is provided a method for preventing, ameliorating or treating a disease or condition selected from obesity or related diseases, conditions; Type II diabetes; atherosclerosis, hypertension; lipidemia, dyslipidemia, microalbuminemia, hyperuricaemia, hypercholesterolemia, hyperlipidemias, hypertriglyceridemias, or combination thereof. In another embodiment, there is provided a method for preventing, ameliorating or treating a disease or condition related to serum levels of triglyceride, LDL, HDL, VLDL, total chlolesterol. In yet another embodiment there is provided a method for preventing, ameliorating or treating a disease or condition selected from obesity or complication thereof, type II diabetes or complication thereof; cardiovascular diseases or complication thereof, or a combination of these.

The compounds of this invention may also be used in conjunction with other active ingredients for the treatment of the diseases, conditions and/or disorders described herein.

Therefore, provided herein is a method for treating a disease or disorder described herein comprising administering concurrently or sequentially one or more compounds described herein with one or more active ingredients known to those skilled in the art. Suitable active ingredients that may be used in combination with the compounds of the present invention include, but are not limited to, anti-obesity agents such as apolipoprotein-B secretion/microsomal triglyceride transfer protein (apo-B/MTP) inhibitors, 11β-hydroxy steroid dehydrogenase-1 (11β-HSD type 1) inhibitors, peptide YY₃₋₃₆ or analogs thereof, MCR-4 agonists, cholecystokinin-A (CCK-A) agonists, monoamine reuptake inhibitors (such as sibutramine), sympathomimetic agents, β₃ adrenergic receptor agonists, dopamine receptor agonists (such as bromocriptine), melanocyte-stimulating hormone receptor analogs, 5HT_{2c} receptor agonists, melanin concentrating hormone antagonists, leptin (the OB protein), leptin analogs, leptin receptor agonists, galanin antagonists, lipase inhibitors (such as tetrahydrolipstatin, i.e. orlistat), anorectic agents (such as a bombesin agonist), neuropeptide-Y receptor antagonists, thyromimetic agents, dehydroepiandrosterone or an analog thereof, glucocorticoid receptor agonists or antagonists, orexin receptor antagonists, glucagon-like peptide-1 (GLP-1) receptor agonists, Protein Tyrosine Phosphatase (PTP-1B) inhibitors, dipeptidyl peptidase IV (DPP-IV) inhibitors, ciliary neurotrophic factors (such as Axokine™ available from Regeneron Pharmaceuticals, Inc., Tarrytown, N.Y. and Procter & Gamble Company, Cincinnati, Ohio), human agouti-related protein (AGRP) inhibitors, ghrelin receptor antagonists, histamine 3 receptor antagonists or inverse agonists, and neuromedin U receptor agonists. Other anti-obesity agents, including the preferred agents set forth herein below, are well known, or will be readily apparent in light of the instant disclosure, to one of ordinary skill in the art.

Antiobesity agents can be selected, for example, from U.S. Pat. Nos. 4,929,629; 3,752,814; 5,274,143; 5,420,305; 5,540,917; 5,643,874; U.S. Publication No. 2002/0141985 and PCT Publication No. WO 03/027637. All of the above recited references are incorporated herein by reference. Especially preferred are anti-obesity agents such as orlistat, sibutramine, bromocriptine, ephedrine, leptin, peptide YY₃₋₃₆ or an analog thereof (including the complete peptide YY), and pseudoephedrine. Preferably, compounds of the present invention and combination therapies are administered in conjunction with exercise and a sensible diet.

The compounds of the present invention may be used alone or in combination with active ingredients in the manufacture of a medicament for the therapeutic applications described herein.

The combination therapy can include one or more of the following embodiments. For example, the one or more active ingredients are selected from antidiabetic agents including, for example, PPARα, PPARγ and/or PPARδ agonists or antagonists (e.g., rosiglitazone, troglitazone or pioglitazone), sulfonylurea drugs (e.g., glyburide, glimepiride, chlorpropamide, tolbutamide or glipizide), non-sulfonylurea secretogogues, α-glucosidase inhibitors (e.g., acrabose, miglitol or voglibose), insulin sensitizers (e.g., PPARγ agonists such as troglitazone, pioglitazone, englitazone, MCC-555, rosiglitazone or other thiazolidinones or no-thiazolidinones; biguanides such as metformine or phetformine; PTP-IB inhibitors; dipeptidyl peptidase IV (DPP-IV) inhibitors), hepatic glucose output lowering compounds (e.g., glucagone antagonists such as glucophage or glucophage XR), insulin and insulin derivatives or a combination thereof.

In another embodiment, the one or more active ingredients are selected from antiobesity drugs including, for example, β-3 agonists, CB receptor modulators (CB1 and/or CB2 receptor modulators such as rimonabant), neuropeptide Y5 inhibitors, ciliary neurotropic factor and derivatives (e.g., axikine), appetite suppressants (e.g., sibutramine), lipase inhibitors (e.g., orlistat) or a combination thereof.

In another embodiment, the one or more active ingredients are selected from HMG COA reductase inhibitors (e.g., lovastatin, simvastatin, pravastatin, fluvastatin, atorvastatin, rivastatin, itavastatin, cerivastatin or ZD-4522), CETP inhibitors (e.g., torcetrapib), lipid lowering drugs, fatty acid lowering compounds, ACAT inhibitors, bile acid sequestrants (e.g., cholestyramine, cholestipol or dextran), bile acid reuptake inhibitors, microsomal triglycerides transport inhibitors, fibric acid derivatives (e.g., clofibrate, fenofibrate, bezafibrate, ciprofibrate, beclofibrate, etofibrate or gemfibrozil), guggle lipids, or a combination thereof.

In yet another embodiment, the one or more active ingredients are selected from antihypertensive drugs including, for example, β-blockers, ACE inhibitors, calcium channel blockers, diuretics, renine inhibitors, AT-1 receptor antagonists, endotheline receptor antagonists and any combination thereof.

### General Methods of Preparation

The compounds described herein, including compounds of general formula I and specific examples, are prepared using techniques known to one skilled in the art through the reaction sequences depicted in Schemes 1-9. Furthermore, in the following schemes, where specific acids, bases, reagents, coupling agents, solvents, etc. are mentioned, it is understood that other suitable acids, bases, reagents, coupling agents etc. may be used and are included within the scope of the present invention. Modifications to reaction conditions, for example, temperature, duration of the reaction or combinations thereof, are envisioned as part of the present invention. The compounds obtained by using the general reaction sequences may be of insufficient purity. These compounds can be purified by using any of the methods for purification of organic compounds known to persons skilled in the art, for example, crystallization or silica gel or alumina column chromatography using different solvents in suitable ratios. All possible stereoisomers are envisioned within the scope of this invention.

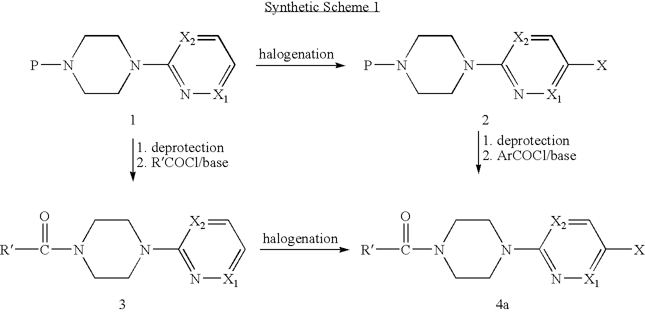

A general approach for the synthesis of intermediates of the general formula 4a is described in Synthetic Scheme 1. The heteroaryl piperazine derivative of the general formula 1 bearing an appropriate protecting group P, for example, t-butoxycarbonyl (BOC), benzyloxycarbonyl (Cbz), benzyl, or 9-fluorenylmethylenoxycarbonyl (Fmoc), is prepared according to methods known in the art, such as that described in Hamlin, K. E. et. al J. Am. Chem. Soc. 1949, 71, 2734-2735. The intermediate of formula 1, wherein X₁ and X₂ is as defined above (e.g., X₁ and X₂ can be N and CH, respectively), is converted to an intermediate of formula 4a, for example, by one of the following two approaches. According to one approach, the intermediate of formula 1 is halogenated (e.g., by treatment with an appropriate halogenating agent (such as N-chlorosuccinimide (NCS), N-bromosuccinimide (NBS), N-iodosuccinimide (NIS), iodine in acetic acid, iodine monochloride, or a mixture thereof)) in a suitable solvent to form the heteroaryl halide of the general formula 2, wherein X is a halogen, such as chlorine, bromine or iodine. The intermediate of formula 2 is deprotected (for example, using standard conditions) to afford the free amine. The amine is acylated to form a compound of formula (4a), for example by reaction with an acid halide (e.g., chloride) (preferably, in the presence of a base). R′ can be a suitably substituted aryl or heteroaryl ring. Alternatively, the intermediate of formula 1 is first deprotected and then acylated to form the intermediate of formula 3, for example, by coupling the deprotected compound with an acid halide (e.g., acid chloride). Upon electrophilic halogenation, the intermediate of formula 3 affords the intermediate of formula 4a.

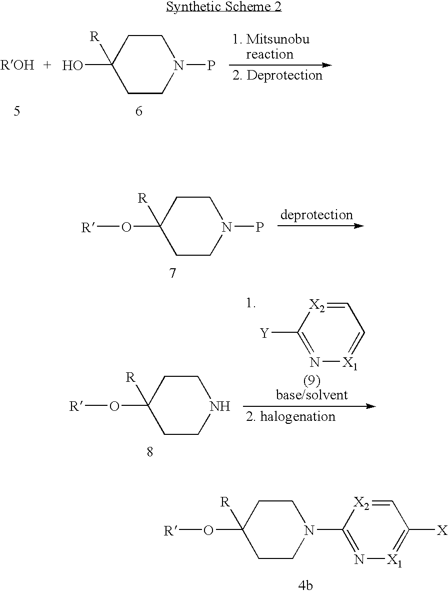

A general approach for the synthesis of aryl ether derivatives of the general formula 4b (where X₁, X₂, R, and R′ are as defined above and X is a halogen) is described in Synthetic Scheme 2. A phenol of the general formula 5 is coupled with an N-protected 4-hydroxypiperidine of the general formula 6, for example, under Mitsunobu reaction conditions, to give an aryl or heteroaryl ether of the general formula 7. The compound of formula 7 is deprotected to form the compound of formula 8. The free base 8 is coupled with an intermediate of general formula 9, where Y is a halogen, preferably under basic or neutral reaction conditions, and the product is halogenated to give the intermediate of formula 4b.

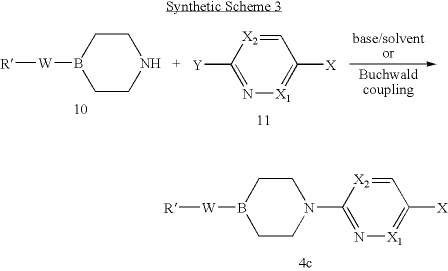

Another general approach for the synthesis of intermediates of the general formula 4c (wherein R′, W, B, X₁, and X₂ are as defined above and X is a halogen) (W is preferably CO, O, CH₂, S(O)ₙ, NH and B is preferably CH, C(R), or N) may be prepared from the intermediate of formula 10 and a 1,4-dihalo heterocycle of the general formula 11 (where X and Y are independently halogen) as shown in Synthetic Scheme 3. The coupling reaction can be carried out using any appropriate organic or inorganic base (such as those described herein) in a suitable organic solvent. Alternatively, a Buchwald coupling reaction can be performed using a palladium catalyst in a suitable organic solvent to afford a compound of formula 4c.

### General Experimental Procedure for Sonogashira Coupling Reaction Described in Synthetic Schemes 4-11:

A general approach for the synthesis of terminal alkynes of the general formula 14 is described in the Synthetic Scheme 4. The compound of formula 14 can be prepared by a Sonogashira coupling reaction followed by hydrolysis.

Aryl or heteroaryl halide 4, where R′, W, B, X₁, and X₂ are as defined earlier and X is a leaving group (such as a halogen), can be converted to an aryl or heteroaryl alkyne of the general formula 14 by two possible approaches using a Sonogashira coupling reaction as the key reaction. In the first approach, the intermediate of formula 4 (where X is halogen and X₁ and X₂ are as defined above) is coupled with 2-methyl-3-butyn-2-ol 12 to afford 13, which is treated with a base, for example, NaH, in a suitable organic solvent to give intermediate 14. Alternatively, a coupling reaction of compound 4 with trimethylsilyl acetylene 15 can be performed to give the trimethylsilyl derivative 16 which is subjected to desilylation, for example with tetrabutyl ammonium fluoride or aqueous NaOH, to afford the compound of formula 14.

For instance, the Sonogashira coupling reaction can be performed as follows. To a stirred solution of alkyne 12 (1.0 mmol) and aryl or heteroaryl halide intermediate 4 (1.0 mmol) in a mixture of triethylamine (1.0-10.0 ml) and dimethylsulfoxide (0-6 ml) is added PdCl₂(PPh₃)₂ (0.01-0.02 mmol) followed by CuI (0.03-0.06 mmol). The mixture is stirred at room temperature to about 80° C. for about 2-24 h under a nitrogen atmosphere. The mixture is diluted with water (50-100 ml) and extracted two to three times with a suitable solvent such as ethyl acetate or chloroform. The combined organic extracts are washed with water and dried over Na₂SO₄. The crude product obtained after evaporation of the solvent can be purified by crystallization from a suitable solvent or by silica gel column chromatography.

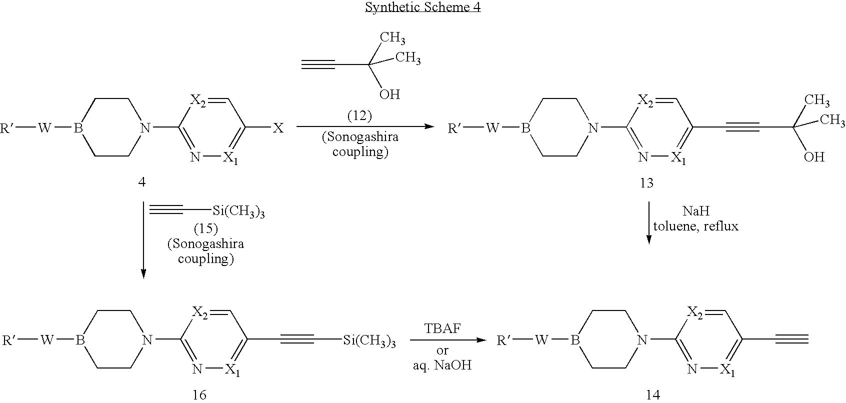

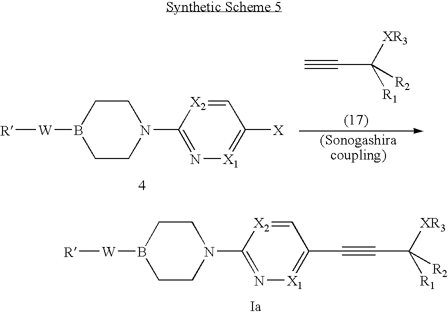

Another general approach for the synthesis of alkynyl derivatives of the general formula Ia, where R′, W, B, X₁, and X₂ are as defined earlier, is shown in Synthetic Scheme 5. The intermediate of formula 4 (where X in formula 4 is a leaving group, such as a halogen) can be converted to the compound of formula Ia by a Sonogashira coupling reaction with a terminal alkyne of the general formula 17, where R₁-R₃ are as defined above and X is as defined above or is O, NHCOR or NHSO₂R (where R is as defined above) (for example, R₁ and R₂ can be independently hydrogen, alkyl, cycloalkyl, arylalkyl, aryl or heteroaryl, X can be O, NHCOR or NHSO₂R (where R is as defined above), and R₃ can be hydrogen, C₁-C₆ alkyl, aryl, alkylaryl, cycloalkyl, or heteroaryl).

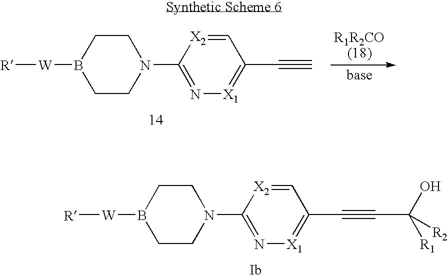

Another approach for the synthesis of 1-substituted 1-hydroxypropynyl derivatives of the general formula Ib (wherein R′, W, B, X₁, X₂, R₁, and R₂ are as defined above) is shown in Synthetic Scheme 6. Treatment of alkynyl derivative 14 with an aldehyde or cyclic or acyclic ketone of the formula 18 in the presence of a suitable base, such as butyllithium, or sodium hydride, in a suitable solvent gives the alcohol of the general formula Ib.

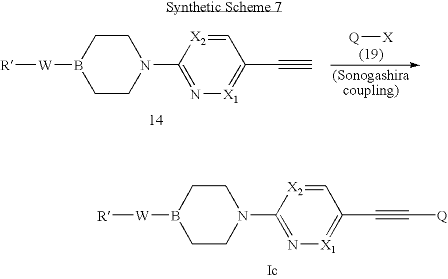

The compounds of the general formula Ic (wherein R′, W, B, X₁, X₂, and Q are as defined above; for example, Q can be alkyl (including hydroxyalkyl), alkenyl, aralkyl, haloalkyl, cycloalkyl (including mono or polysubstituted cycloalkyl), aryl (including mono or polysubstituted aryl), aryloxyalkyl, heteroaryl or heteroaryloxyalkyl) may be prepared as shown in Synthetic Scheme 7, using a Sonogashira coupling reaction. For example, the coupling reaction of the intermediate of formula 14 with a halide of the general formula 19, where X is a leaving group (such as a halogen, e.g., chlorine, bromine or iodine) and Q is as defined above, can be carried out in the presence of a palladium-phosphine ligand complex and a catalytic amount of copper(I) salt or a silver(I) oxide, preferably in the presence of a large excess of an organic amine with or without an organic solvent (for a review see: Chinchilla, R.; Nájera, C. Chemical Reviews 2007, 107(3), pp 874-922) to afford a compound of the general formula Ic. Suitable palladium catalysts include, for example, Pd(OAc)₂, PdCl₂, [(Ph)₃P]₂PdCl₂, Cl₂Pd(PPh₃)₂, and Pd(PPh₃)₄. A variety of reaction conditions may be employed for the coupling reaction.

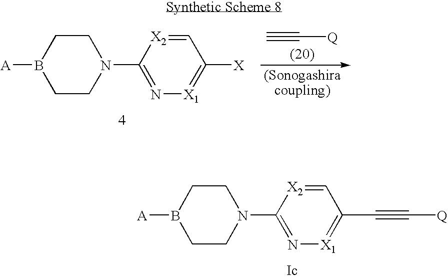

An alternative general approach using the above coupling reaction is shown in Synthetic Scheme 8 (wherein A, B, X₁, and X₂ are as defined above). Reaction of aryl or heteroaryl compound 4 (where X is a leaving group, such as a halogen) and terminal alkyne of the general formula 20, for example, under Sonogashira coupling reaction conditions, affords the compound of the formula Ic.

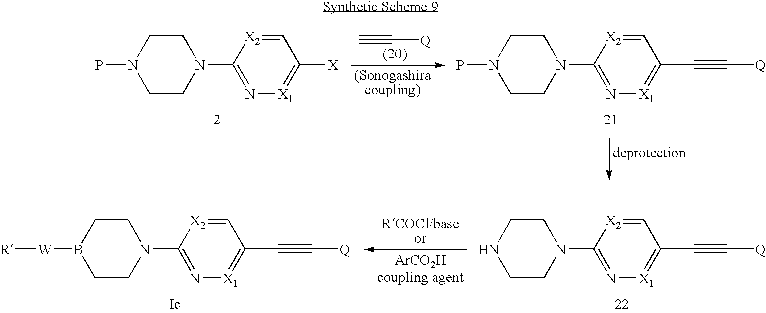

The compounds of the present invention of the general formula Ic, can also be prepared by appropriate modification of the synthetic sequence. One such approach is given in Synthetic Scheme 9. The intermediate of formula 2, where X, P, X₁ and X₂ are as defined above, is reacted with a terminal alkyne of the general formula 20 to give the intermediate of formula 21 which on deprotection affords the amine 22. The amine 22 is converted to compound Ic. For example, amine 22 is reacted with a compound of the formula R′COX (where X is a leaving group) (e.g., an appropriate aryl or heteroaryl carboxylic acid), preferably in the presence of a suitable coupling agent to form compound Ic. Alternatively, the amine 22 is reacted with an acid chloride, preferably in the presence of a suitable base, to form a compound of general formula Ic.

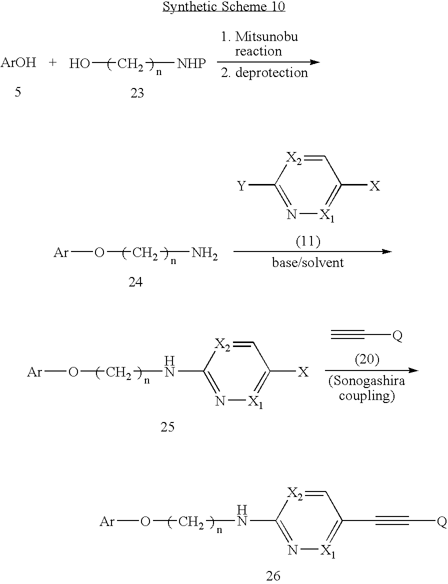

A general approach for the synthesis of the general formula 26 is shown in the Scheme 10. A phenol of the general formula 5 is coupled with an N-protected amino alcohol of the general formula 23 (where n is 2 to 5), preferably under Mitsunobu reaction conditions, followed by deprotection to give an aryl or heteroaryl ether of the general formula 24. The free base 24 is coupled with an intermediate of general formula 11 where X and Y are independently halogen, preferably under basic conditions, to give intermediate 25. The intermediate of formula 25 is coupled with a terminal alkyne derivative of the general formula 20, for example, under Sonogashira reaction conditions, to afford a compound of the general formula 26.

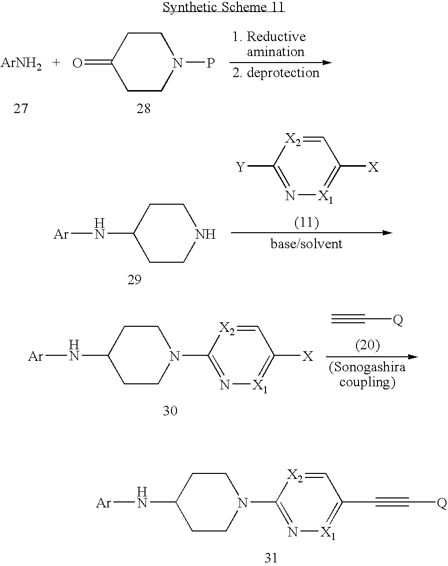

A general approach for the synthesis of compound of the general formula 31 is shown in Synthetic Scheme 11. An aryl or heteroaryl amine of the general formula 27 is treated with an N-protected-4-piperidinone (28) under reductive amination conditions, followed by deprotection to give an intermediate of the general formula 29. The free base 29 is coupled with an intermediate of general formula 11 where X and Y are independently halogen, preferably under basic conditions, to give the intermediate of formula 30. The intermediate of formula 30 is coupled with a terminal alkyne derivative of the general formula 20, preferably under Sonogashira reaction conditions, to afford a compound of the general formula 31.

The starting materials for synthetic schemes 1-11 are commercially available or can be prepared according to methods known in the art. The synthetic schemes disclosed above are only specific approaches for preparing the compounds of the invention and persons skilled in the art may be able to prepare these intermediates and the compounds of the invention using alternative synthetic routes and approaches. More specific details of synthetic methods are given below.

### EXPERIMENTAL

### Intermediate 1: 4-(5-Iodo-2-pyridyl)piperazino-2-trifluoromethylphenylmethanone

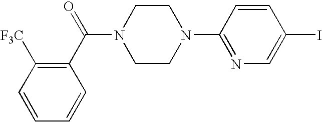

Step 1: 1-Pyridin-2-ylpiperazine: To a stirred solution of piperazine (10.8 g, 126.581 mmol) in pyridine (10 ml) was added 2-bromopyridine (10.0 g, 63.293 mmol) and the mixture was stirred at 150° C. for 7 h under nitrogen atmosphere. The mixture was cooled to room temperature, diluted with water (200 ml) and ethyl acetate (200 ml). The layers were separated. The aqueous layer was extracted with ethyl acetate (2×100 ml). The combined organic extracts were washed with water (2×100 ml), followed by brine (100 ml). The solution was evaporated under reduced pressure to give viscous brown oil, which on fractional distillation at 120° C. and 1.4 mm Hg pressure gave 14.3 g of the product as an oil; ¹H NMR (300 MHz, CDCl₃) δ 1.97 (s, 1H), 2.97-3.00 (m, 4H), 3.48-3.51 (m, 4H), 6.60-6.65 (m, 2H), 7.47 (t, J=7.2 Hz, 1H), 8.19 (d, J=3 Hz, 1H).

Step 2: tert-Butyl 4-pyridin-2-ylpiperazine-1-carboxylate: To a stirred solution of Step 1 intermediate (3.0 g, 18.414 mmol) in acetonitrile (10 ml) was added a solution of di-tert-butyl dicarbonate (6.0 g, 27.613 mmol) in acetonitrile (10 ml). The mixture was stirred at room temperature for 18 h under nitrogen atmosphere. The solvent was distilled off under reduced pressure and the residue obtained was triturated with n-pentane to give 4.5 g of the product as a white solid; ¹H NMR (300 MHz, CDCl₃) δ 1.48 (s, 9H), 3.53 (br s, 8H), 6.63-6.66 (m, 2H), 7.50 (t, J=6.9 Hz, 1H), 8.19 (d, J=3.3 Hz, 1H).

Step 3: tert-Butyl 4-(5-iodopyridin-2-yl)piperazine-1-carboxylate: To a stirred solution of Step 2 intermediate (2.5 g, 9.588 mmol) in carbon tetrachloride (25 ml) was added N-iodosuccinimide (3.3 g, 14.663 mmol) and benzoyl peroxide (92 mg, 0.38 mmol) and the mixture was stirred overnight at ambient temperature under nitrogen atmosphere. The mixture was diluted with water and extracted with chloroform (2×50 ml). The combined organic extracts were washed with saturated sodium bisulfite solution (2×50 ml), water (2×100 ml) followed by brine (50 ml). The crude product obtained after evaporation of the solvent was purified by silica gel column chromatography using 10% EtOAc in petroleum ether to give 3.1 g of the product as a white solid; ¹H NMR (300 MHz, CDCl₃) δ 1.48 (s, 9H), 3.50 (br s, 8H), 6.48 (d, J=9.3 Hz, 1H), 7.68 (d, J=8.7 Hz, 1H), 8.31 (s, 1H).

Step 4: 1-(5-Iodo-2-pyridyl)piperazine: Step 3 intermediate (3.0 g, 7.712 mmol) was treated with 15% HCl in EtOAc (10 ml) at 10° C. for 30 min and the reaction mixture was evaporated under reduced pressure to result a white solid. The hydrochloride salt thus obtained was dissolved in water (10 ml) and the pH was adjusted to 13 with solid K₂CO₃. The mixture was extracted with chloroform (3×30 ml) and combined chloroform extracts were dried (Na₂SO₄) and evaporated to give 2.1 g of the product as a white solid.

Step 5: 4-(5-Iodo-2-pyridyl)piperazino-2-trifluoromethylphenylmethanone: To a stirred solution of Step 4 intermediate (2.0 g, 6.920 mmol) in dichloromethane (15 ml) was added 2-(trifluoromethyl)benzoic acid (1.58 g 8.304 mmol), N-(3-dimethylaminopropyl)-N¹-ethylcarbodiimide hydrochloride (1.60 g, 10.381 mmol), 1-hydroxybenzotriazole hydrate (1.05 g, 6.921 mmol) followed by triethylamine (1.74 g, 17.303 mmol). The clear solution was stirred at room temperature for 4 h under nitrogen atmosphere. Water (30 ml) was added and the mixture was extracted with dichloromethane (2×100 ml). The combined organic extracts were washed with water (2×100 ml) and brine (50 ml). This crude product obtained after evaporation of the solvent was purified by silica gel column chromatography using 30% EtOAc in chloroform to give 2.9 g of the product as a white solid; IR (KBr) 2918, 1645, 1435, 1241, 1009, 769 cm⁻¹; ¹H NMR (300 MHz, CDCl₃) δ 3.26-3.32 (m, 2H), 3.44-3.55 (m, 2H), 3.58-3.45 (m, 2H), 3.82-4.02 (m, 2H), 6.50 (d, J=8.7 Hz, 1H), 7.35 (d, J=6.6 Hz, 1H), 7.55-7.75 (m, 4H), 8.32 (s, 1H); ESI-MS (m/z) 462.73 (M+H)⁺.

### Intermediate 2: 4-[5-(1-Ethynyl)-2-pyridinyl]piperazino-2-trifluoromethylphenyl-methanone

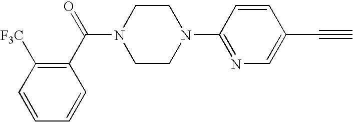

Step 1: 2-Trifluoromethylphenyl-4-[5-(2-trimethylsilyl-1-ethynyl)pyridin-2-yl]piperazino methanone: To a stirred solution of Intermediate 1 (6.0 g, 13.015 mmol) in triethylamine (60 ml) was added PdCl₂(PPh₃)₂ (180 mg, 0.246 mmol) followed by CuI (147 mg, 0.753 mmol). The mixture was stirred for 10 min and (trimethylsilyl)acetylene (1.89 g, 19.506 mmol) was added and stirred at room temperature for 18 h. The mixture was diluted with water (50 ml) and extracted with chloroform (2×50 ml). The combined organic layer was washed with water (2×100 ml) followed by brine (50 ml). The solution was concentrated under vacuum to give 5.6 g of the product as a white solid which was used as such for next step.

Step 2: 4-[5-(1-Ethynyl)-2-pyridinyl]piperazino-2-trifluoromethylphenylmethanone: To a stirred solution of Step 1 intermediate (3.0 g, 6.964 mmol) in methanol (10 ml) was added 1N NaOH (12 ml) and the mixture was stirred at room temperature for 2 h. Water (30 ml) was added and the mixture was extracted with chloroform (2×100 ml). The combined organic layer was washed with water (2×100 ml), brine (50 ml) and concentrated under vacuum to afford crude product. This crude product was purified by silica gel column chromatography using 10% EtOAc in chloroform to give 2.1 g of the product as an off-white solid; IR (KBr) 2858, 2105, 1646, 1499, 1245, 1006, 771 cm⁻¹; ¹H NMR (300 MHz, CDCl₃) δ 3.08 (s, 1H), 3.28 (br s, 2H), 3.55 (br s, 2H), 3.69 (br s, 2H), 3.88-4.01 (m, 2H), 6.58 (d, J=9.0 Hz, 1H), 7.36 (d, J=7.5 Hz, 1H), 7.55-7.70 (m, 3H), 7.74 (d, J=7.8 Hz, 1H), 8.30 (s, 1H); ESI-MS (m/z) 360.37 (M+H)⁺.

### Intermediate 3: 4-(5-Iodo-2-pyridyl)piperazino-2,5-dichlorophenylmethanone

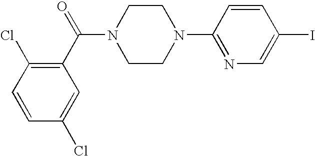

Coupling reaction of 1-(5-Iodo-2-pyridyl)piperazine (4.0 g, 13.840 mmol) with 2,5-dichlorobenzoic acid (3.17 g, 16.607 mmol) in the presence of EDCI (3.98 g, 29.481 mmol), HOBT (2.12 g, 13.856 mmol) and triethylamine (3.49 g, 34.554 mmol) in dichloromethane (150 ml) as described in Intermediate 1, Step 5 followed by purification by silica gel column chromatography using 30% EtOAc in chloroform gave 6.2 g of the product as a white solid; ¹H NMR (300 MHz, CDCl₃) δ 3.20-3.65 (m, 6H), 3.80-4.01 (m, 2H), 6.51 (d, J=9.0 Hz, 1H), 7.31-7.35 (m, 3H), 7.71 (d, J=9.0 Hz, 1H), 8.33 (s, 1H).

### Intermediate 4: 2,5-Dichlorophenyl-4-[5-(1-ethynyl)-2-pyridyl]piperazinomethanone

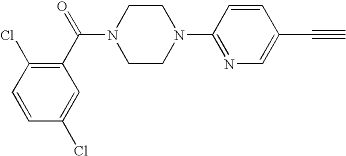

This compound was prepared as described in Intermediate 2 from Intermediate 3 (4.0 g, 8.657 mmol) and (trimethylsilyl)acetylene (1.27 g, 12.982 mmol) followed by base assisted desilylation to give 4.3 g of the product as an off-white solid; ¹H NMR (300 MHz, CDCl₃) δ 3.09 (s, 1H), 3.32-3.90 (m, 2H), 3.63-3.70 (m, 4H), 3.85-4.01 (m, 2H), 6.53 (d. J=9.0 Hz, 1H), 7.31-7.38 (m, 3H), 7.58 (d, J=9.0 Hz, 1H), 8.31 (s, 1H).

### Intermediate 5: 4-(6-Iodo-3-pyridazinyl)piperazino-2-trifluoromethylphenylmethanone

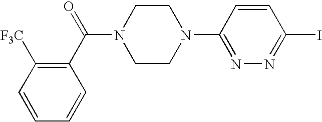

Step 1: tert-Butyl 4-(6-iodopyridazin-3-yl)piperazine-1-carboxylate: A mixture of 3,6-diiodopyridazine (8.0 g, 24.169 mmol), N—BOC-piperazine (6.51 g, 35.113 mmol) and KHCO₃ (6.09 g, 60.243 mmol) in dry DMF (200 ml) was stirred at 80° C. for 48 h under nitrogen atmosphere. The mixture was cooled to room temperature, diluted with water (40 ml) and extracted with EtOAc (2×100 ml). The combined extracts were washed with water (2×200 ml) and dried over Na₂SO₄. The crude product obtained after evaporation of the solvent was purified by silica gel column chromatography using 15% EtOAc in chloroform to give 9.3 g of the product as a white solid; ¹H NMR (300 MHz, CDCl₃) δ 1.48 (s, 9H), 3.60-3.65 (m, 8H), 6.64 (d, J=9.6 Hz, 1H), 7.49 (d, J=9.6 Hz, 1H); ESI-MS (m/z) 391.18 (M+H)⁺.

Step 2: 1-(6-Iodo-3-pyridazinyl)piperazine: Trifluoroacetic acid (27 ml) was added to a stirred and cooled (10° C.) solution of Step 1 intermediate (9.0 g, 23.136 mmol) in dry dichloromethane (27 ml). The mixture was stirred at same temperature for 30 min under nitrogen atmosphere. Excess trifluoroacetic acid and dichloromethane were distilled off under reduced pressure to give a viscous residue. The residue was dissolved in water (50 ml) and the solution was basified to pH 13 with solid K₂CO₃. The solid precipitated out was filtered and dried to give 6.2 g of the product as a white solid.

Step 3: 4-(6-Iodo-3-pyridazinyl)piperazino-2-trifluoromethylphenylmethanone: To a stirred and cooled (10° C.) solution of Step 2 intermediate (6.0 g, 20.687 mmol) in dry dichloromethane (60 ml) was added triethylamine (3.1 g, 30.693 mmol) and 2-(trifluoromethyl)benzoyl chloride (4.32 g, 20.717 mmol) under nitrogen atmosphere for 30 min. The mixture was diluted with water (100 ml) and extracted with chloroform (2×100 ml). The combined organic extracts were washed with water (2×200 ml) and dried over anhydrous Na₂SO₄. The solvent was evaporated under reduced pressure to give 8.7 g of the product as a white solid; ¹H NMR (300 MHz, CDCl₃) δ 3.33 (t, J=5.1 Hz, 2H), 3.60-3.72 (m, 4H), 3.86-4.06 (m, 2H), 6.94 (d, J=9.6 Hz, 1H), 7.27 (d, J=8.7 Hz, 1H), 7.36 (d, J=7.2 Hz, 1H), 7.54-7.66 (m, 2H), 7.75 (d, J=7.8 Hz, 1H); ESI-MS (m/z) 463.33 (M+H)⁺.

### Intermediate 6: 4-[6-(1-Ethynyl)-3-pyridazinyl]piperazino-2-trifluoromethylphenyl-methanone

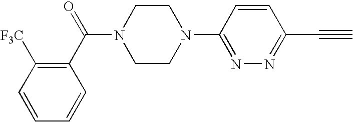

This compound was prepared by the Sonogashira coupling reaction of the Intermediate 4 (5.0 g, 10.822 mmol) with (trimethylsilyl)acetylene (2.40 g, 10.822 mmol) in a mixture of triethylamine (8 ml) and dry DMSO (50 ml) followed by desilylation using 1N NaOH (30 ml) to give 2.43 g of the product as an off-white solid; ¹H NMR (300 MHz, CDCl₃) δ 3.27 (s, 1H), 3.32-3.35 (m, 2H), 3.70-4.10 (m, 6H), 6.85 (d, J=9.6 Hz, 1H), 7.36 (d, J=9.6 Hz, 2H), 7.56-7.66 (m, 2H), 7.75 (d, J=7.2 Hz, 1H).

### Intermediate 7: 4-(5-Iodo-2-pyrimidinyl)piperazino-2-trifluoromethylphenylmethanone

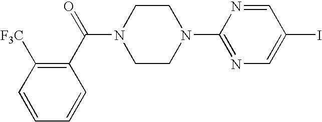

Step 1: tert-Butyl 4-(2-pyrimidinyl)-1-piperazine carboxylate: A mixture of 2-chloropyrimidine (6.18 g, 54.051 mmol), N—BOC-piperazine (10.0 g, 54.051 mmol) and KHCO₃ (11.10 g, 80.432 mmol) in dry DMF (100 ml) was stirred at 80° C. for 18 h under nitrogen atmosphere. Extractive work up using chloroform followed by silica gel column chromatography using 15% EtOAc in chloroform gave 8.8 g of the product as a white solid; ¹H NMR (300 MHz, CDCl₃) δ 1.48 (s, 9H), 3.49 (br s, 4H), 3.80 (br s, 4H), 6.51 (br s, 1H), 8.32 (d, J=4.2 Hz, 2H).

Step 2: tert-Butyl 4-(5-iodo-2-pyrimidinyl)-1-piperazine carboxylate: Iodination of Step 1 intermediate (8.0 g, 30.426 mmol) with N-iodosuccinimide (10.3 g, 45.784 mmol) in the presence of 50% benzoyl peroxide (220 mg, 0.454 mmol) in CCl₄ (160 ml) as described in Intermediate 1, Step 3 for 7 days followed by chromatographic purification using 10% EtOAc in petroleum ether gave 10.9 g of the product as a white solid; ¹H NMR (300 MHz, CDCl₃) δ 1.48 (s, 9H), 3.46-3.49 (m, 4H), 3.74-3.77 (m, 4H), 8.39 (s, 2H).

Step 3: 5-Iodo-2-piperazinopyrimidine: The Step 2 intermediate (10.0 g, 25.70 mmol) was deprotected with trifluoroacetic acid (30 ml) in dry dichloromethane (30 ml) and the product was isolated as the free base as described in Intermediate 5, Step 2 to give 6.3 g of the product which was used as such for the next step.

Step 4: 4-(5-Iodo-2-pyrimidinyl)piperazino-2-trifluoromethylphenylmethanone: Coupling reaction of Step 3 intermediate (5.8 g, 20.076 mmol) with 2-(trifluoromethyl)benzoyl chloride (4.2 g, 20.147 mmol) in the presence of triethylamine (3.05 g, 30.195 mmol) in dry dichloromethane (40 ml) as described in Intermediate 5, Step 3 to give 8.1 g of the product as a white solid; ¹H NMR (300 MHz, CDCl₃) δ 3.20-3.24 (m, 2H), 3.69-3.72 (m, 2H), 3.75-3.96 (m, 4H), 7.35 (d, J=7.5 Hz, 1H), 7.51-7.64 (m, 2H), 7.73 (d, J=8.1 Hz, 1H), 8.39 (s, 2H).

### Intermediate 8: 4-[5-(1-Ethynyl)-2-pyrimidinyl]piperazino-2-trifluoromethylphenyl-methanone

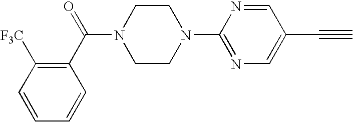

### Method A:

This compound was prepared by the Sonogashira coupling reaction of the Intermediate 5 (3.0 g, 6.505 mmol) with (trimethylsilyl)acetylene (958 mg, 9.753 mmol) in a mixture of triethylamine (8 ml) and dry DMSO (50 ml) followed by desilylation using 1N NaOH (20 ml) to give 1.7 g of the product as an off-white solid; ¹H NMR (300 MHz, CDCl₃) δ 3.19 (s, 1H), 3.22-3.26 (m, 2H), 3.78-4.03 (m, 6H), 7.36 (d, J=7.5 Hz, 1H), 7.53-7.72 (m, 2H), 7.73 (d, J=7.5 Hz, 1H), 8.40 (s, 2H); ESI-MS (m/z) 361.17 (M+H)⁺.

### Method B:

Step 1: 4-[5-(3-Hydroxy-3-methyl-1-butynyl)-2-pyrimidinyl]piperazino-2-trifluoromethylphenylmethanone: To a stirred solution of Intermediate 7 (5.0 g, 10.845 mmol) and 2-methyl-but-3-yn-2-ol (1.8 g, 21.697 mmol) in TEA (50 ml) was added PdCl₂(PPh₃)₂ (76 mg, 0.108 mmol) followed by CuI (61 mg, 0.011 mmol). The mixture was stirred at room temperature for 18 h. The mixture was diluted with water (100 ml) and extracted with chloroform (2×100 ml). The combined organic layer was washed with water (2×100 ml) followed by brine (100 ml). The solvent was evaporated under reduced pressure to give 3.7 g of the product as an off-white solid; ¹H NMR (300 MHz, CDCl₃) δ 1.61 (s, 6H), 2.09 (br s, 1H, exchangeable with D₂O), 3.24 (t, J=5.1 Hz, 2H), 3.72-4.08 (m, 6H), 7.36 (d, J=7.2 Hz, 1H), 7.60-7.70 (m, 2H), 7.73 (d, J=7.8 Hz, 1H), 8.34 (s, 2H); ESI-MS (m/z) 419.19 (M+H)⁺.

Step 2: 4-[5-(1-Ethynyl)-2-pyrimidinyl]piperazino-2-trifluoromethylphenylmethanone: To the stirred suspension of Step 1 intermediate (3.0 g, 11.978 mmol) in toluene (50 ml), sodium (198 mg, 8.612 mmol) was added and refluxed for 30 min under nitrogen atmosphere. The reaction mixture was cooled to room temperature, quenched with dry methanol (3 ml) and diluted with water (30 ml). The mixture was extracted with EtOAc (2×50 ml) and the combined extracts were washed with water (2×50 ml). The organic extracts were dried (Na₂SO₄) and evaporated under reduced pressure to give 1.9 g of the product as an off-white solid which showed identical IR and ¹H NMR spectra to the product obtained by Method A.

### Intermediate 9: 4-[5-(1-Ethynyl)-1,3-thiazol-2-yl]piperazino-2-trifluoromethylphenyl methanone

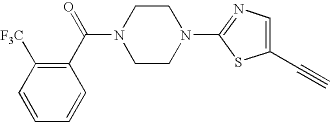

Step 1: tert-Butyl 4-(1,3-thiazol-2-yl)-1-piperazine carboxylate: To a stirred solution of 2-bromothiazole (5.0 g, 30.482 mmol) and N—BOC-piperazine (5.49 g, 30.482 mmol) in DMF (50 ml) was added K₂CO₃ (8.42 g, 60.965 mmol) and the mixture was stirred at 80° C. for 4 days under nitrogen atmosphere. The mixture was cooled to room temperature and diluted with water (100 ml) and EtOAc (100 ml). The layers were separated and the aqueous layer was extracted with EtOAc (30 ml). The combined organic phase was washed with water (3×100 ml) and dried over Na₂SO₄. The crude product obtained after evaporation of the solvent was purified by silica gel column chromatography using 15% EtOAc in petroleum ether to give 3.2 g of the product as an off-white solid; ¹H NMR (300 MHz, CDCl₃) δ 1.48 (s, 9H), 3.45-3.48 (m, 4H), 3.55-3.58 (m, 4H), 6.60 (d, J=3.6 Hz, 1H), 7.20 (d, J=3.6 Hz, 1H); ESI-MS (m/z) 270.33 (M+H)⁺.

Step 2: tert-Butyl 4-(5-iodo-1,3-thiazol-2-yl)-1-piperazine carboxylate: This compound was prepared by iodination of Step 1 intermediate (2.23 g, 8.293 mmol) with N-iodosuccinimide (2.79 g, 12.440 mmol) in the presence of 50% benzoyl peroxide (200 mg, 0.829 mmol) in CCl₄ (20 ml) for 2 h under nitrogen atmosphere. Extractive work up followed by recrystallization of the crude product from EtOAc gave 3.14 g of the product as an off-white solid; ¹H NMR (300 MHz, CDCl₃) δ 1.48 (s, 9H), 3.41-3.43 (m, 4H), 3.55 (br s, 4H), 7.08 (s, 1H); ESI-MS (m/z) 396.38 (M+H)⁺.

Step 3: 5-Iodo-2-piperazino-1,3-thiazole: To a stirred solution of Step 2 intermediate (3.14 g, 7.941 mmol) was deprotected using trifluoroacetic acid and the free base was isolated as described in Intermediate 5, Step 2 to give 2.26 g of the product as a white solid which was used as such for the next step.

Step 4: 4-(5-Iodo-1,3-thiazol-2-yl)piperazino-2-trifluoromethylphenylmethanone: Coupling reaction of Step 3 intermediate (2.24 g, 7.593 mmol) with 2-(trifluoromethyl)benzoyl chloride (1.58 g, 7.593 mmol) in the presence of triethylamine (2.30 g, 22.779 mmol) in dry dichloromethane (30 ml) as described in Intermediate 5, Step 3 to give 3.9 g of the product as an off-white solid; ¹H NMR (300 MHz, CDCl₃) δ 3.30-3.32 (m, 2H), 3.38-3.42 (m, 2H), 3.52-3.57 (m, 2H), 3.87-4.01 (m, 2H), 7.20 (s, 1H), 7.35 (d, J=7.2 Hz, 1H), 7.55-7.63 (m, 2H), 7.74 (d, J=7.5 Hz, 1H); ESI-MS (m/z) 468.66 (M+H)⁺.

Step 5: 4-[5-(1-Ethynyl)-1,3-thiazol-2-yl]piperazino-2-trifluoromethylphenyl methanone: This compound was prepared by the Sonogashira coupling reaction of Step 3 intermediate (500 mg, 1.070 mmol) with (trimethylsilyl)acetylene (157 mg, 1.605 mmol) in dichloromethane (10 ml) followed by tetra-n-butylammonium fluoride (TBAF) assisted desilylation to give 422 mg of the product as an off-white solid; IR (KBr) 2863, 2196, 1631, 1500, 1314, 1007, 766 cm⁻¹; ¹H NMR (300 MHz, CDCl₃) δ 3.25-3.36 (m, 3H), 3.40-3.50 (m, 2H), 3.59 (q, J=4.5, 5.1 Hz, 2H), 3.82-3.92 (m, 1H), 4.00-4.10 (m, 1H), 7.35 (d, J=9.0 Hz, 2H), 7.50-7.70 (m, 2H), 7.74 (d, J=7.5 Hz, 1H); ESI-MS (m/z) 366.54 (M+H)⁺.

### Intermediate 10: 3-[4-(Cyclopentylcarbonyl)piperazin-1-yl]-6-ethynylpyridazine

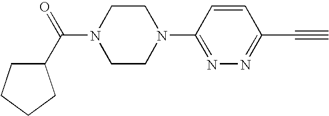

Step 1: tert-Butyl 4-(cyclopentylcarbonyl)piperazine-1-carboxylate: To a stirred solution of cyclopentanecarboxylic acid (2.0 g, 17.52 mmol) in dichloromethane (50 ml) was added N—BOC-piperazine (4.73 g, 26.25 mmol), EDCI (3.55 g, 26.25 mmol), HOBT (4.02 g, 26.25 mmol) followed by triethylamine (5.31 g, 52.56 mmol). The mixture was stirred at room temperature for 18 h under nitrogen atmosphere. Water (50 ml) was added and the mixture was extracted with chloroform (2×200 ml). The combined organic layer was washed with water (2×300 ml), brine (100 ml) and dried over Na₂SO₄. The crude product obtained after evaporation of the solvent was purified by silica gel column chromatography using 15% EtOAc in chloroform to give 5.5 g of the product as a white solid; IR (KBr) 2963, 2291, 1687, 1424, 1230, 1025, 768 cm⁻¹; ¹H NMR (300 MHz, CDCl₃) δ 1.44 (s, 9H), 1.50-1.90 (m, 8H), 2.78-2.95 (m, 1H), 3.36-3.56 (m, 6H), 3.58-3.65 (m, 2H); ESI-MS (m/z) 283.10 (M+H)⁺.

Step 2: 1-(Cyclopentylcarbonyl)piperazine: To a stirred solution of Step 1 intermediate (5.0 g, 17.730 mmol) in dichloromethane (15 ml) was added TFA (15 ml) at 10° C. The mixture was stirred at same temperature for 30 min and the mixture was evaporated to dryness to give the product as its TFA salt. The free base was obtained by basification (pH 12-13) followed by extractive work up to give 3.1 g of the product as a white solid which was used as such for the next step.

Step 3: 3-[4-(Cyclopentylcarbonyl)piperazin-1-yl]-6-iodopyridazine: Coupling reaction of Step 2 intermediate (3.0 g, 17.018 mmol) with 3,6-diiodopyridazine (5.65 g, 17.018 mmol) in dry DMF (50 ml) in the presence of KHCO₃ as described in Intermediate 5, Step 1 gave 2.73 g of the product as an off-white solid; IR (KBr) 2951, 2344, 1638, 1427, 1235, 1022, 833 cm⁻¹; ¹H NMR (300 MHz, CDCl₃) δ 1.52-1.92 (m, 8H), 2.84-3.00 (m, 1H), 3.51 (br s, 2H), 3.66 (br s, 2H), 3.76 (br s, 4H), 6.64 (d. J=9.3 Hz, 1H), 7.49 (d, J=9.3 Hz, 1H); ESI-MS (m/z) 387.03 (M+H)⁺.

Step 4: 3-[4-(Cyclopentylcarbonyl)piperazin-1-yl]-6-ethynylpyridazine: This compound was prepared by the Sonogashira coupling reaction of Step 3 intermediate (1.3 g, 3.410 mmol) with (trimethylsilyl)acetylene (0.537 g, 5.467 mmol) in the presence of PdCl₂(PPh₃)₂ (24 mg, 0.032 mmol) and CuI (19 mg, 0.102 mmol) in a mixture of triethylamine (5 ml) and DMSO (20 ml) followed by tetra-n-butylammonium fluoride (TBAF) assisted desilylation to give 900 mg of the product as an off-white solid; IR (KBr) 2939, 2111, 1628, 1428, 1234, 1023, 921 cm⁻¹; ¹H NMR (300 MHz, CDCl₃) δ 1.50-1.92 (m, 8H), 2.84-3.00 (m, 1H), 3.26 (s, 1H), 3.50-3.64 (m, 2H), 3.68 (br s, 2H), 3.76-3.84 (m, 2H), 3.84-3.95 (m, 2H), 6.82 (d. J=9.9 Hz, 1H), 7.33 (d, J=9.3 Hz, 1H); ESI-MS (m/z) 285.26 (M+H)⁺.

### Intermediate 11: 3-[4-(Cyclopropylmethyl)piperazin-1-yl]-6-iodopyridazine

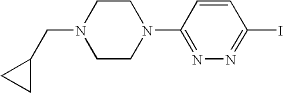

Step 1: tert-Butyl 4-(cyclopropylmethyl)piperazine-1-carboxylate: Prepared by alkylation of N—BOC-piperazine (2.0 g, 11.102 mmol) with (bromomethyl)cyclopropane (1.79 g, 13.322 mmol) in the presence of K₂CO₃ (2.30 g, 16.641 mmol) as base in DMF (20 ml) to give 2.03 g of the product as a colorless liquid; IR (KBr) 2977, 1694, 1422, 1246, 1167, 1006, 759 cm⁻¹; ¹H NMR (300 MHz, CDCl₃) δ 0.05 (d, J=5.1 Hz, 2H), 0.44 (d, J=8.4 Hz, 2H), 0.80 (br s, 1H), 1.36 (s, 9H), 2.16 (d, J=6.3 Hz, 2H), 2.30-2.40 (m, 4H), 3.29 (br s, 4H); ESI-MS (m/z) 241.63 (M+H)⁺.

Step 2: 1-(Cyclopropylmethyl)piperazine: Deprotection of Step 1 intermediate (2.0 g, 8.368 mmol) with TFA (6 ml) in dichloromethane (6 ml) followed by basic work up of the reaction mixture as described in Intermediate 5, Step 2 gave 1.21 g of the product as a white solid which was used as such for the next step.

Step 3: 3-[4-(Cyclopropylmethyl)piperazin-1-yl]-6-iodopyridazine: Coupling reaction of Step 2 intermediate (1.0 g, 7.142 mmol) with 3,6-diiodopyridazine (2.37 g, 7.142 mmol) in the presence of KHCO₃ (1.07 g, 10.714 mmol) in DMF (30 ml) at 80° C. followed by chromatographic purification (3% MeOH in chloroform) of the crude material gave 706 mg of the product as an off-white solid; IR (KBr) 2912, 1619, 1571, 1432, 1260, 1156, 920 cm⁻¹; ¹H NMR (300 MHz, CDCl₃) δ 0.17 (br s, 2H), 0.59 (br s, 2H), 0.95 (br s, 1H), 2.38 (br s, 2H), 2.71 (br s, 4H), 3.71 (br s, 4H), 6.61 (d, J=9.0 Hz, 1H), 7.45 (d, J=9.9 Hz, 1H); ESI-MS (m/z) 345.97 (M+H)⁺.

### Intermediate 12: 3-[4-(Cyclohexylmethyl)piperazin-1-yl]-6-iodopyridazine

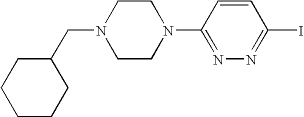

Prepared in 3 steps from cyclohexylmethyl bromide, N—BOC-piperazine and 3,6-diiodopyridazine as described in Intermediate 11 to give the product as a white solid; IR (KBr) 2915, 1569, 1421, 1249, 1127, 835 cm⁻¹; ¹H NMR (300 MHz, CDCl₃) δ 0.87 (q, J=11.1 Hz, 3H), 1.12-1.30 (m, 4H), 1.42-1.60 (m, 1H), 1.70-1.84 (m, 3H), 2.15 (d. J=6.9 Hz, 2H), 2.48 (t, J=4.8 Hz, 4H), 3.59 (t, J=5.4 Hz, 4H), 6.59 (d, J=7.5 Hz, 1H), 7.42 (d, J=9.6 Hz, 1H); ESI-MS (m/z) 387.19 (M+H)⁺.

### Intermediate 13: 3-[4-(2-Fluorobenzyl)piperazin-1-yl]-6-iodopyridazine

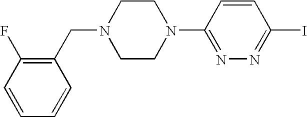

Prepared in 3 steps from 2-fluorobenzyl bromide, N—BOC-piperazine and 3,6-diiodopyridazine as described in Intermediate 11 to give the product as a white solid; IR (KBr) 2842, 1572, 1433, 1242, 1148, 758 cm⁻¹; ¹H NMR (300 MHz, CDCl₃) δ 2.44-2.64 (m, 4H), 3.50-3.70 (m, 6H), 6.59 (d, J=9.9 Hz, 1H), 6.94-7.16 (m, 2H), 7.18-7.30 (m, 1H), 7.32-7.50 (m, 2H); ESI-MS (m/z) 399.68 (M+H)⁺.

### Intermediate 14: 3-Ethynyl-6-[4-(2-fluorobenzyl)piperazin-1-yl]pyridazine

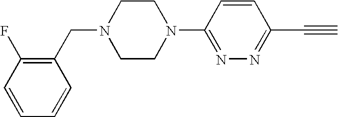

Prepared by the Sonogashira coupling reaction of Intermediate 13 with (trimethylsilyl)acetylene in triethylamine followed by desilylation as described in Intermediate 2 to give the product as an off-white solid; IR (KBr) 2847, 2345, 1582, 1434, 1226, 1001, 761 cm⁻¹; ¹H NMR (300 MHz, CDCl₃) δ 2.50-2.69 (m, 4H), 3.22 (s, 1H), 3.64 (s, 2H), 3.68-3.78 (m, 4H), 6.76 (d, J=9.3 Hz, 1H), 6.98-7.18 (m, 2H), 7.20-7.30 (m, 2H), 7.37 (t, J=7.8 Hz, 1H); ESI-MS (m/z) 297.47 (M+H)⁺.

### Intermediate 15: 4-Benzyl-1-(6-iodopyridazin-3-yl)piperidin-4-ol

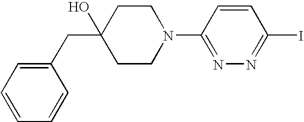

Step 1: tert-Butyl 4-benzyl-4-hydroxypiperidine-1-carboxylate: tert-butyl 4-oxo-piperidine-1-carboxylate (5.0 g, 25.641 mmol) in dry diethyl ether (50 ml) was added over 5 min to a stirred and cooled (0° C.) solution of benzylmagnesium bromide (5.0 g, 25.641 mmol) in diethyl ether. The mixture was allowed to warm to room temperature and further stirred for 2 h. The reaction mixture was quenched with saturated aqueous ammonium chloride solution (100 ml) and extracted by EtOAc (2×100 ml). The combined organic extracts were washed with water (100 ml) and dried over Na₂SO₄. The product obtained after evaporation of the solvent was purified by silica gel column chromatography using 10% EtOAc in petroleum ether to give 6.1 g of the product as a white solid; ¹H NMR (300 MHz, CDCl₃) δ 1.44 (s, 9H), 1.46 (br s, 1H, D₂O exchangeable), 1.65-1.75 (m, 4H), 2.75 (s, 2H), 2.80-2.96 (m, 4H), 7.17 (d, J=6.3 Hz, 2H), 7.22-7.35 (m, 3H).

Step 2: 4-Benzyl-4-hydroxypiperidine: Deprotection of Step 1 intermediate (5.0 g, 17.241 mmol) using TFA (15 ml) in dichloromethane (15 ml) followed by basic work up of the reaction mixture as described in Intermediate 5, Step 5 gave 2.9 g of the product as a white solid which was used as such for the next step.

Step 3: 4-Benzyl-1-(6-iodopyridazin-3-yl)piperidin-4-ol: Coupling reaction of Step 2 intermediate (2.0 g, 10.526 mmol) with 3,6-diiodopyridazine (3.49 g, 10.526 mmol) in the presence of KHCO₃ (3.49 g, 10.526 mmol) in DMF (25 ml) at 70° C. followed by extractive work up and chromatographic purification (15% EtOAc in petroleum ether) gave 1.52 g of the product as an off-white solid; IR (KBr) 2936, 1571, 1422, 1259, 1132, 837 cm⁻¹; ¹H NMR (300 MHz, CDCl₃) δ 1.64 (br s, 1H, exchangeable with D₂O), 1.58-1.70 (m, 4H), 2.77 (s, 2H), 3.31 (t, J=13.2 Hz, 2H), 4.09 (d, J=13.2 Hz, 2H), 6.62 (d, J=8.7 Hz, 1H), 7.17 (d, J=7.5 Hz, 2H), 7.20-7.38 (m, 3H), 7.40 (d, J=9.3 Hz, 1H).

### Intermediate 16: 4-(2-Fluorobenzyl)-1-(6-iodopyridazin-3-yl)piperidin-4-ol

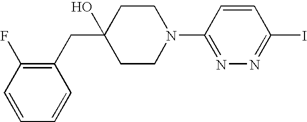

Prepared in 3 steps as described in the synthesis of Intermediate 15 from 2-fluorobenzylmagnesium bromide, tert-butyl 4-oxo-piperidine-1-carboxylate and 3,6-diiodopyridazine to give the product as an off-white solid; ¹H NMR (300 MHz, CDCl₃) δ 1.64 (br s, 1H, exchangeable with D₂O), 1.56-1.70 (m, 2H), 1.72-1.84 (m, 2H), 2.84 (s, 2H), 3.33 (t, J=12.3 Hz, 2H), 4.09 (d, J=13.2 Hz, 2H), 6.62 (d, J=9.9 Hz, 1H), 7.00-7.16 (m, 3H), 7.18-7.30 (m, 1H), 7.40 (d, J=9.3 Hz, 1H).

### Intermediate 17: 4-(2,5-Dichlorobenzyl)-1-(6-iodopyridazin-3-yl)piperidin-4-ol

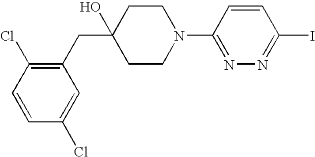

Prepared in 3 steps as described in the synthesis of Intermediate 15 from 2,5-dichlorobenzylmagnesium bromide, tert-butyl 4-oxo-piperidine-1-carboxylate and 3,6-diiodopyridazine to give the product as an off-white solid; IR (KBr) 2946, 2348, 1576, 1245, 1089, 966 cm⁻¹; ¹H NMR (300 MHz, CDCl₃) δ 1.66 (br s, 1H, exchangeable with D₂O), 1.58-1.74 (m, 2H), 1.78-1.90 (m, 2H), 2.95 (s, 2H), 3.24-3.40 (m, 2H), 4.12 (d, J=13.8 Hz, 2H), 6.63 (d, J=9.9 Hz, 1H), 7.15 (dd, J=6.3, 2.4 Hz, 1H), 7.25-7.40 (m, 2H), 7.41 (d, J=9.3 Hz, 1H); ESI-MS (m/z) 464.25 (M)⁺.

### Intermediate 18: 3-[3-(2-Fluorophenoxy)azetidin-1-yl]-6-iodopyridazine

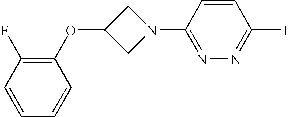

Step 1: 1-(Diphenylmethyl)-3-(2-fluorophenoxy)azetidine: To a stirred mixture of 2-fluorophenol (4.76 g, 42.543 mmol) and NaH (1.53 g, 63.756 mmol) in dimethylacetamide (50 ml) was added 1-(diphenylmethyl)azetidin-3-ylmethanesulfonate (13.5 g, 42.543 mmol) and the mixture was maintained at 80° C. for 12 h under nitrogen atmosphere. The reaction mixture was cooled, quenched with water (20 ml) and diluted with EtOAc (50 ml). The layers were separated and the organic layer was washed with water (30 ml) and dried over anhydrous Na₂SO₄. The solvent was evaporated under reduced pressure to give 7.2 g of the product as a white solid; IR (KBr) 2945, 2218, 1611, 1503, 1260, 1062, 751 cm⁻¹; ¹H NMR (300 MHz, CDCl₃) δ 3.15 (t, J=7.2 Hz, 2H), 3.70 (t, J=7.2 Hz, 2H), 4.42 (s, 1H), 4.75-4.84 (m, 1H), 6.65 (t, J=8.1 Hz, 1H), 6.75-7.08 (m, 3H), 7.12-7.29 (m, 6H), 7.38 (d, J=7.2 Hz, 4H); ESI-MS (m/z) 334.12 (M+H)⁺.

Step 2: 3-(2-Fluorophenoxy)azetidine: Hydrogenolysis of Step 1 intermediate (7.0 g, 2.102 mmol) with Pd(OH)₂ in methanol at 40 psi H₂ gas pressure for 2 h gave 6.5 g of product as a semisolid which was used as such for the next step.

Step 3: 3-[3-(2-Fluorophenoxy)azetidin-1-yl]-6-iodopyridazine: Coupling reaction of Step 2 intermediate (2.0 g, 11.963 mmol) in DMF (25 ml) with 3,6-diiodopyridazine (3.97 g, 11.966 mmol) in the presence of KHCO₃ (1.79 g, 17.938 mmol) at 80° C. for 12 h under nitrogen atmosphere as described in Intermediate 5, Step 1 gave 1.12 g of the product as an off-white solid; IR (KBr) 2940, 2323, 1581, 1463, 1263, 1040, 827 cm⁻¹; ¹H NMR (300 MHz, CDCl₃) δ 4.20-4.30 (m, 2H), 4.50-4.58 (m, 2H), 5.15 (br s, 1H), 6.29 (d, J=9.3 Hz, 1H), 6.75 (t, J=8.7 Hz, 1H), 6.90-7.16 (m, 3H), 7.46 (d, J=9.3 Hz, 1H); ESI-MS (m/z) 372.17 (M+H)⁺.

### Intermediate 19: 3-[(3S)-3-(2-Fluorophenoxy)azolan-1-yl]-6-iodopyridazine

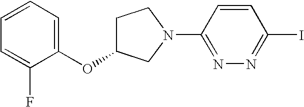

Step 1: tert-Butyl (3S)-3-(2-fluorophenoxy)azolan-1-carboxylate: To a stirred solution of tert-butyl-(3R)-3-hydroxyazolane-1-carboxylate (3.0 g, 16.032 mmol) in dry THF (25 ml) was added triphenylphosphine (6.03 g, 24.048 mmol), 2-fluorophenol (1.79 g, 15.98 mmol) followed by diethyl azodicarboxylate (3.63 g, 20.835 mmol). The mixture was stirred at room temperature for 18 h under nitrogen atmosphere. The crude product obtained after evaporation of the solvent was purified by column chromatography using 100-200 mesh silica gel and 10% EtOAc in chloroform to give 2.66 g of the product as a white solid; IR (KBr) 2980, 2287, 1682, 1478, 1240, 833 cm⁻¹; ¹H NMR (300 MHz, CDCl₃) δ 1.46 (s, 9H), 2.07 (br s, 1H), 2.19 (br s, 1H), 3.48-3.80 (m, 4H), 4.81 (br s, 1H), 6.92-7.10 (m, 4H).

Step 2: (3S)-3-(2-Fluorophenoxy)azolane: Deprotection of Step 1 intermediate (2.6 g, 9.242 mmol) with TFA (8 ml) in dichloromethane (8 ml) followed by basic work up as described in Intermediate 5, Step 2 to give 1.5 g of the product as a white solid which was used for the next step.

Step 3: 3-[(3S)-3-(2-Fluorophenoxy)azolan-1-yl]-6-iodopyridazine: Coupling reaction of Step 2 intermediate (1.5 g, 8.275 mmol) with 3,6-diiodopyridazine (2.74 g, 8.276 mmol) in the presence of KHCO₃ (1.25 g, 12.367 mmol) in DMF (70 ml) as described in Intermediate 5, Step 1 followed by chromatographic purification using 15% EtOAc in chloroform gave 1.7 g of the product as an off-white solid; IR (KBr) 2930, 2233, 1582, 1255, 749 cm⁻¹; ¹H NMR (300 MHz, CDCl₃) δ 2.24-2.28 (m, 1H), 2.42 (br s, 1H), 3.66-3.92 (m, 4H), 5.09 (br s, 1H), 6.39 (d, J=9.3 Hz, 1H), 6.92-7.09 (m, 4H), 7.43 (d, J=9.3 Hz, 1H); ESI-MS (m/z) 386.13 (M+H)⁺.

### Intermediate 20: 3-(1-Ethynyl)-6-[(3S)-3-(2-fluorophenoxy)azolan-1-yl]pyridazine

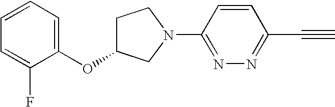

Prepared by the Sonogashira coupling of Intermediate 19 with (trimethylsilyl)acetylene in triethylamine followed by base assisted desilylation as described in Intermediate 2 gave the product as an off-white solid; ¹H NMR (300 MHz, CDCl₃) δ 2.25-2.31 (m, 1H), 2.40-2.47 (m, 1H), 3.16 (s, 1H), 3.74-4.00 (m, 4H), 5.10 (s, 1H), 6.57 (d, J=9.3 Hz, 1H), 6.93-7.10 (m, 4H), 7.29 (d, J=9.3 Hz, 1H).

### Intermediate 21: 4-(2-Fluorophenoxy)-1-(5-iodo-2-pyridyl)piperidine

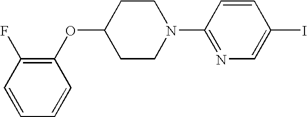

Step 1: 1-(2-Pyridyl)-4-piperidinol: To a stirred solution of 4-hydroxypiperidine (15.05 g, 94.936 mmol) in pyridine (30 ml) was added 2-bromopyridine (10.0 g, 63.297 mmol) and the mixture was stirred at 155° C. for 18 h under nitrogen atmosphere. The mixture was cooled to room temperature, diluted with water (200 ml) and ethyl acetate (200 ml). The layers were separated. The aqueous layer was extracted with EtOAc (2×100 ml). The combined organic extracts were washed with water (2×100 ml) followed by brine (100 ml). The solution was evaporated under reduced pressure to give viscous brown oil, which was purified by silica gel column chromatography using 3% methanol in chloroform to give 8.91 g of the product as a yellow oil; IR (KBr) 2940, 1597, 1484, 1228, 1075, 756 cm⁻¹; ¹H NMR (300 MHz, CDCl₃) δ 1.56-1.63 (m, 2H), 1.96-1.98 (m, 2H), 1.72 (br s, 1H), 3.08-3.17 (m, 2H), 3.87-3.92 (m, 1H), 4.03-4.08 (m, 2H), 6.55-6.59 (m, 1H), 6.65 (d, J=8.4 Hz, 1H), 7.41-7.46 (m, 1H), 8.14-8.16 (m, 1H); ESI-MS (m/z) 179.40 (M+H)⁺.

Step 2: 1-(5-Iodo-2-pyridyl)-4-piperidinol: To a stirred solution of Step 1 intermediate (8.9 g, 50.183 mmol) in CCl₄ (25 ml) was added N-iodosuccinimide (16.87 mg, 75.00 mmol) and 50% benzoyl peroxide (1.6 g, 4.995 mmol). The mixture was stirred at room temperature for 24 h under nitrogen atmosphere. The mixture was diluted with water (100 ml) and extracted with chloroform (2×100 ml). The combined organic layers were washed with saturated sodium bisulfite solution (2×50 ml), water (2×100 ml) followed by brine (50 ml). The crude product obtained after evaporation of the solvent on recrystallization from acetone gave 10.2 g of the product as a white solid; IR (KBr) 2923, 1578, 1220, 1078, 805 cm⁻¹; ¹H NMR (300 MHz, CDCl₃) δ 1.50-1.68 (m, 2H), 1.71 (br s, 1H), 1.90-2.08 (m, 2H), 3.17 (t, J=10.2 Hz, 2H), 3.89-4.06 (m, 3H), 6.50 (d, J=8.7 Hz, 1H), 7.62 (dd, J=6.9, 2.4 Hz, 1H), 8.27 (s, 1H); ESI-MS (m/z) 305.57 (M+H)⁺.

Step 3: 4-(2-Fluorophenoxy)-1-(5-iodo-2-pyridyl)piperidine: To a stirred solution of Step 2 Intermediate (2.0 g, 10.309 mmol) in dry THF (25 ml) was added triphenylphosphine (4.05 g, 15.463 mmol), 2-fluorophenol (1.15 g, 10.309 mmol) followed by diethyl azodicarboxylate (2.33 g, 13.401 mmol). The mixture was stirred at room temperature for 30 min then heated to 60-65° C. for 3 h under nitrogen atmosphere. The crude product obtained after evaporation of the solvent was purified by column chromatography using 100-200 mesh silica gel and 5% EtOAc in petroleum ether to give 1.6 g of the product as a white solid; IR (KBr) 3068, 2948, 1575, 1258, 1034, 748 cm⁻¹; ¹H NMR (300 MHz, CDCl₃) δ 1.83-1.89 (m, 2H), 1.97-2.03 (m, 2H), 3.38-3.46 (m, 2H), 3.83-3.90 (m, 2H), 4.48-4.50 (m, 1H), 6.51 (d, J=9.3 Hz, 1H), 6.91-7.10 (m, 4H), 7.62 (d, J=9.3 Hz, 1H), 8.27 (s, 1H); ESI-MS (m/z) 399.27 (M+H)⁺.

### Intermediate 22: 5-Ethynyl-2-[4-(2-fluorophenoxy)piperidin-1-yl]pyridine

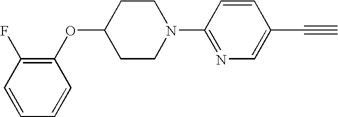

To a stirred solution of Intermediate 21 (1.3 g, 3.266 mmol) and (trimethysilyl)acetylene (481 mg, 4.899 mmol) in triethylamine (20 ml) was added PdCl₂(PPh₃)₂ (46 mg, 0.653 mmol) followed by CuI (37 mg, 0.195 mmol). The mixture was stirred at 80° C. for 6 h under nitrogen atmosphere. Water (50 ml) was added and the mixture was extracted with ethyl acetate (2×50 ml). The combined organic layer was filtered through celite and was washed with water (4×100 ml) followed by brine (50 ml) and dried (Na₂SO₄) and concentrated in vacuum to afford crude product. Tetra-n-butylammonium fluoride trihydrate (1.78 g, 5.664 mmol) was added to the crude product in dichloromethane (20 ml) and stirred at room temperature for 30 min. The mixture was diluted with water (50 ml) and extracted with dichloromethane (2×50 ml). The combined organic layer was washed with water (2×40 ml) and dried over anhydrous Na₂SO₄. The crude material obtained after evaporation of the solvent on chromatographic purification using 10% EtOAc in chloroform gave 1.5 g of the product as an off-white solid; IR (KBr) 2952, 2099, 1601, 1500, 1234, 1023, 925 cm⁻¹; ¹H NMR (300 MHz, CDCl₃) δ 1.82-1.98 (m, 2H), 1.98-2.10 (m, 2H), 3.06 (s, 1H), 3.42-3.58 (m, 2H), 3.84-4.05 (m, 2H), 4.51 (br s, 1H), 6.58 (d, J=8.7 Hz, 1H), 6.85-7.16 (m, 4H), 7.50 (d, J=9.3 Hz, 1H), 8.28 (s, 1H); ESI-MS (m/z) 297.59 (M)⁺.

### Intermediate 23: 3-[4-(2-Fluorophenoxy)piperidin-1-yl]-6-iodopyridazine

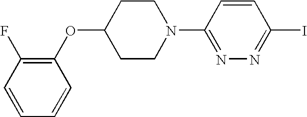

Step 1: tert-Butyl 4-(2-fluorophenoxy)piperidine-1-carboxylate: Mitsunobu coupling reaction of N—BOC-4-hydroxypiperidine (5.0 g, 24.854 mmol) with 2-fluorophenol (2.78 g, 24.85 mmol) in the presence of triphenylphosphine (9.77 g, 37.285 mmol) and diethyl azodicarboxylate (5.62 g, 32.267 mmol) in dry THF (50 ml) as described in Intermediate 19 followed by silica gel column chromatography using 10% EtOAc in petroleum ether gave 7.1 g of the product as a viscous liquid; ¹H NMR (300 MHz, CDCl₃) δ 1.42 (s, 9H), 1.72-1.80 (m, 2H), 1.82-1.94 (m, 2H), 3.23-3.31 (m, 2H), 3.67-3.70 (m, 2H), 4.40 (br s, 1H), 6.89-7.07 (m, 4H).

Step 2: 4-(2-Fluorophenoxy)piperidine: Deprotection of Step 1 intermediate (7.0 g, 23.725 mmol) with trifluoroacetic acid (21 ml) in dichloromethane (21 ml) followed by basic work up of the mixture as described Intermediate 5, Step 2 gave 4.5 g of the product as a viscous liquid.

Step 3: 3-Iodo-6-[4-(2-fluorophenoxy)piperidino]pyridazine: Coupling reaction of Step 2 intermediate (4.4 g, 22.56 mmol) with 3,6-diiodopyridazine (7.49 g, 22.56 mmol) in the presence of KHCO₃ (3.42 g, 33.84 mmol) in DMF as described in Intermediate 5, Step 1 followed by work up and chromatographic purification (15% EtOAc in petroleum ether) gave 6.2 g of the product as an off-white solid; ¹H NMR (300 MHz, CDCl₃) δ 1.91-2.06 (m, 4H), 2.57-2.65 (m, 2H), 3.89-3.94 (m, 2H), 4.54-4.57 (m, 1H), 6.68 (d, J=9.6 Hz, 1H), 6.94-7.13 (m, 4H), 7.46 (d, J=9.6 Hz, 1H); ESI-MS (m/z) 399.35 (M+H)⁺.

### Intermediate 24: 3-Ethynyl-6-{4-[2-(fluorophenoxy)piperidin-1-yl]}pyridazine

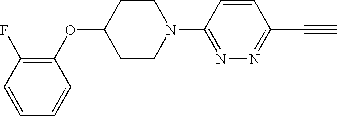

Prepared in the similar manner as described in the preparation of Intermediate 2 by Sonogashira coupling of Intermediate 23 with (trimethylsilyl)acetylene followed by base assisted desilylation to give the product as an off-white solid; IR (KBr) 2946, 1598, 1501, 1259, 1021, 748 cm⁻¹; ¹H NMR (300 MHz, DMSO-d₆) δ 1.60-1.72 (m, 2H), 1.98-2.20 (m, 2H), 3.48-3.55 (m, 2H), 4.03-4.10 (m, 2H), 4.40 (s, 1H), 4.69 (br s, 1H), 6.94-7.00 (m, 1H), 7.11-7.29 (m, 3H), 7.30 (d, J=9.3 Hz, 1H), 7.49 (d, J=9.3 Hz, 1H); ESI-MS (m/z) 297.65 (M+H)⁺.

### Intermediate 25: 3-[4-(4-Bromo-2-fluorophenoxy)piperidino]-6-(1-ethynyl)pyridazine

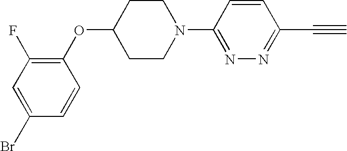

Step 1: 3-[4-(4-Bromo-2-fluorophenoxy)piperidin-1-yl]-6-iodopyridazine Prepared in 3 steps from 4-bromo-2-fluorophenol, tert-butyl-4-hydroxypiperidine-1-carboxylate and 3,6-diiodopyridazine as described in Intermediate 23 to give the product as an off-white solid; IR (KBr) 2928, 2403, 1575, 1491, 1260, 1020, 869 cm⁻¹; ¹H NMR (300 MHz, CDCl₃) δ 1.89-2.10 (m, 4H), 3.59-3.62 (m, 2H), 3.86-4.00 (m, 2H), 4.52 (br s, 1H), 6.66 (d, J=9.3 Hz, 1H), 6.88 (t, J=8.7 Hz, 1H), 7.19-7.27 (m, 2H), 7.45 (d, J=8.7 Hz, 1H); ESI-MS (m/z) 478.26 (M)⁺.

Step 2: 3-[4-(4-Bromo-2-fluorophenoxy)piperidino]-6-(1-ethynyl)pyridazine: Prepared by a Sonogashira coupling of Step 1 intermediate with (trimethylsilyl)acetylene followed by desilylation as described in Intermediate 9, Step 5 to give the product as an off-white solid; IR (KBr) 2955, 2233, 1589, 1496, 1264, 1022, 805 cm⁻¹; ¹H NMR (300 MHz, CDCl₃) δ 1.90-2.05 (m, 4H), 3.24 (s, 1H), 3.66-3.74 (m, 2H), 3.93-4.00 (m, 2H), 4.53 (br s, 1H), 6.82-6.92 (m, 2H), 7.16-7.31 (m, 3H); ESI-MS (m/z) 376.42 (M)⁺.

### Intermediate 26: 3-(1-Ethynyl)-6-[4-{2-(trifluoromethyl)phenoxy}piperidino]pyridazine

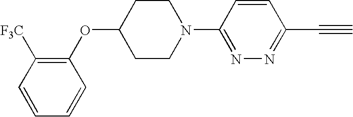

Prepared in 4 steps in the similar manner as described in the above intermediates from tert-butyl 4-hydroxypiperidine-1-carboxylate, 2-trifluoromethylphenol and 3,6-diiodopyridazine to afford the product as an off-white solid; IR (KBr) 2946, 2233, 1590, 1459, 1320, 1117, 758 cm⁻¹; ¹H NMR (300 MHz, CDCl₃) δ 2.04 (d, J=4.5 Hz, 4H), 3.26 (s, 1H), 3.76-3.84 (m, 2H), 3.96-4.00 (m, 2H), 4.81 (br s, 1H), 6.87 (d, J=9.6 Hz, 1H), 7.03-7.05 (m, 2H), 7.33 (d, J=9.6 Hz, 1H), 7.51 (t, J=7.8 Hz, 1H), 7.62 (d, J=7.5 Hz, 1H); ESI-MS (m/z) 348.31 (M)⁺.

### Intermediate 27: 2-{1-[6-Ethynyl)-3-pyridazinyl]-4-piperidyloxy}benzonitrile

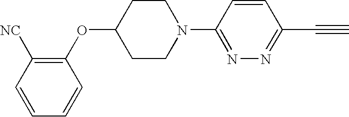

Prepared in 4 steps in the similar manner as described in the above intermediates from tert-butyl 4-hydroxypiperidine-1-carboxylate, 2-cyanophenol and 3,6-diiodopyridazine to give the product as an off-white solid; IR (KBr) 2945, 2227, 1595, 1452, 1287, 1008, 759 cm⁻¹; ¹H NMR (300 MHz, CDCl₃) δ 2.03 (br s, 4H), 3.24 (s, 1H), 3.88-3.92 (m, 4H), 4.75-4.78 (m, 1H), 6.85 (d, J=9.0 Hz, 1H), 7.02-7.05 (m, 2H), 7.29 (d, J=9.9 Hz, 1H), 7.49-7.59 (m, 2H); ESI-MS (m/z) 305.24 (M+H)⁺.

### Intermediate 28: 3-[4-(2,5-Dichlorophenoxy)piperidin-1-yl]-6-iodopyridazine

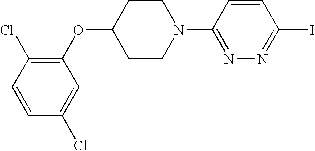

Prepared in 4 steps in the similar manner as described in the above intermediates from tert-butyl 4-hydroxypiperidine-1-carboxylate, 2,5-dichlorophenol and 3,6-diiodo pyridazine to give the product as an off-white solid; IR (KBr) 2979, 1569, 1426, 1231, 1038, 916 cm⁻¹; ¹H NMR (300 MHz, CDCl₃) δ 1.90-2.10 (m, 4H), 3.70-3.90 (m, 4H), 4.62 (br s, 1H), 6.65 (d, J=9.0 Hz, 1H), 6.88-6.98 (m, 2H), 7.27 (d, J=7.8 Hz, 1H), 7.50 (d, J=10.2 Hz, 1H).

### Intermediate 29: 3-Ethynyl-6-[4-(pyridin-3-yloxy)piperidin-1-yl]pyridazine

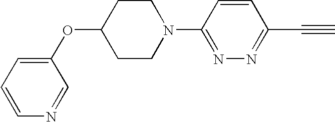

Prepared in 4 steps in the similar manner as described in the above intermediates from tert-butyl 4-hydroxypiperidine-1-carboxylate, 3-hydroxypyridine and 3,6-diiodo pyridazine to give the product as an off-white solid; IR (KBr) 2944, 2233, 1578, 1482, 1261, 1023, 839 cm⁻¹; ¹H NMR (300 MHz, CDCl₃) δ 1.84-2.00 (m, 2H), 2.02-2.16 (m, 2H), 3.24 (s, 1H), 3.64-3.78 (m, 2H), 3.90-4.05 (m, 2H), 4.63 (br s, 1H), 6.84 (d, J=9.9 Hz, 1H), 7.10-7.30 (m, 2H), 7.29 (d, J=9.9 Hz, 1H), 8.21 (s, 1H), 8.31 (s, 1H); ESI-MS (m/z) 281.23 (M+H)⁺.

### Intermediate 30: 5-(1-Ethynyl)-2-[4-(2-fluorophenoxy)piperidino]pyrimidine

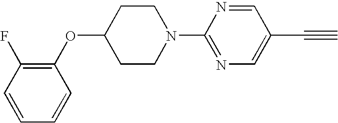

Step 1: 1-(2-Pyrimidinyl)-4-piperidinol: This compound was prepared according to procedure described in Intermediate 21, Step 1 by a coupling reaction of 2-chloropyrimidine (15 g, 49.504 mmol) with 4-hydroxypiperidine (5.7 g, 49.504 mmol) in presence of KHCO₃ (9.9 g, 98.881 mmol) in DMF (150 ml) to give 8.8 g of the product as a yellow oily liquid; ¹H NMR (300 MHz, CDCl₃) δ 1.47-1.59 (m, 2H), 1.93-1.98 (m, 3H), 3.5-3.34 (m, 2H), 3.91-3.98 (m, 1H), 4.38-4.45 (m, 2H), 6.46 (t, J=4.8 Hz, 1H), 8.30 (d, J=4.8 Hz, 2H); ESI-MS (m/z) 180.19 (M+H)⁺.

Step 2: 1-(5-Iodo-pyrimidin-2-yl)-4-piperidinol: The Step 1 intermediate (8.7 g, 48.603 mmol) was iodinated as described in Intermediate 21, Step 2 with N-iodosuccinimide (16.35 g, 7.669 mmol) in the presence of 50% benzoyl peroxide (2.34 g, 9.66 mmol) in CCl₄ (150 ml) to give 5.2 g of the product as an off-white solid. ¹H NMR (300 MHz, CDCl₃) δ 1.46-1.58 (m, 3H), 1.91-1.97 (m, 2H), 3.28-3.36 (m, 2H), 3.93-3.98 (m, 1H), 4.28-4.36 (m, 2H), 8.37 (s, 2H); ESI-MS (m/z) 306.29 (M+H)⁺.

Step 3: 2-[4-(2-Fluorophenoxy)piperidino]-5-iodopyrimidine: Mitsunobu coupling of Step 2 intermediate (2.0 g, 6.55 mmol) with 2-fluorophenol (735 mg, 6.55 mmol) in presence of PPh₃ (2.87 g, 9.82 mmol) and DEAD (1.7 g, 9.755 mmol) in dry THF (25 ml) gave 1.02 g of the product as a colorless oil; ¹H NMR (300 MHz, CDCl₃) δ 1.84-2.00 (m, 4H), 3.65-3.73 (m, 2H), 4.08-4.15 (m, 2H), 4.53 (br s, 1H), 6.94-7.13 (m, 4H), 8.34 (s, 2H); ESI-MS (m/z) 400.51 (M+H)⁺.

Step 4: 5-(1-Ethynyl)-2-[4-(2-fluorophenoxy)piperidino]pyrimidine: This compound was prepared in the same manner as described in Intermediate 2 by a Sonogashira coupling of Step 3 intermediate (1.0 g, 2.506 mmol) and (trimethylsilyl)acetylene (369 mg, 3.756 mmol) in presence of CuI (28 mg, 0.147 mmol), PdCl₂(PPh₃)₂ (35 mg, 0.0498 mmol) in triethylamine (10 ml) followed by base assisted desilylation to give 400 mg of the product as an off-white solid; ¹H NMR (300 MHz, CDCl₃) δ 1.88-2.02 (m, 4H), 3.18 (s, 1H), 3.78-3.87 (m, 2H), 4.14-4.18 (m, 2H), 4.57 (br s, 1H), 6.95-7.10 (m, 4H), 8.40 (s, 2H); ESI-MS (m/z) 298.19 (M+H)⁺.

### Intermediate 31: N-[2-(2-Fluorophenoxy)ethyl]-6-iodopyridazin-3-amine

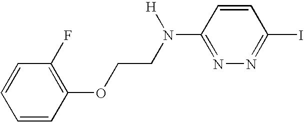

Prepared in 3 steps in the similar manner as described in the preparation of previous intermediates from tert-butyl (2-hydroxyethyl)carbamate, 2-fluorophenol and 3,6-diiodopyridazine to give the product as an off-white solid; ¹H NMR (300 MHz, CDCl₃) δ 3.84-3.95 (m, 2H), 4.20-4.30 (m, 2H), 5.11 (br s, 1H), 6.44 (d, J=9.0 Hz, 1H), 6.84-7.10 (m, 4H), 7.40 (d, J=9.0 Hz, 1H). ESI-MS (m/z) 360.82 (M+H)⁺.

### Intermediate 32: N-(2-Fluorophenyl)-1-(6-iodopyridazin-3-yl)piperidin-4-amine

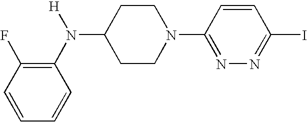

Step 1: tert-Butyl[4-(2-fluorophenyl)amino]piperidine-1-carboxylate: To a stirred solution of tert-butyl 4-oxopiperidine-1-carboxylate (4.0 g, 20.050 mmol) in EDC (50 ml) was added 2-fluoroaniline (2.23 g, 20.050 mmol) followed by sodium triacetoxyborohydride (8.52 g, 40.201 mmol) at room temperature. Acetic acid (1.33 g, 11.055 mmol) was added to this mixture and stirred the mixture overnight at the same temperature. The mixture was basified to pH 10 and extracted with chloroform (2×50 ml). The combined organic layer was washed with water (100 ml) and dried over anhydrous Na₂SO₄. The crude product obtained after evaporation of the solvent was purified by silica gel column chromatography using 10% EtOAc in petroleum ether to afford 2.4 g of the product as an off-white solid; ¹H NMR (300 MHz, CDCl₃) δ 1.44 (s, 9H), 3.54 (br s, 2H), 4.07 (br s, 2H), 5.08 (br s, 1H), 6.84-7.00 (m, 2H), 7.00-7.10 (m, 2H).

Step 2: N-(2-Fluorophenyl)piperidin-4-amine: Deprotection of Step 1 intermediate (2.0 g, 6.802 mmol) using TFA (6 ml) followed by basic work up as described in Intermediate 5, Step 2 gave 1.2 g of the product as a brown sticky liquid which was used as such for the next step.

Step 3: N-(2-Fluorophenyl)-1-(6-iodopyridazin-3-yl)piperidin-4-amine: Coupling of Step 2 intermediate (1.0 g, 5.154 mmol) with 3,6-diiodopyridazine (1.7 g, 5.154 mmol) in the presence of KHCO₃ (0.77 g, 7.731 mmol) in DMF (10 ml) as described in Intermediate 5, Step 1 gave 1.72 g of the product as a white solid; IR (KBr) 3413, 2928, 2837, 1618, 1433, 1244, 1033, 745 cm⁻¹; ¹H NMR (300 MHz, CDCl₃) δ 1.44-1.69 (m, 2H), 2.17 (d, J=14.1 Hz, 2H), 3.17 (t, J=12.6 Hz, 2H), 3.58 (br s, 1H), 3.78 (br s, 1H), 4.25 (d, J=13.8 Hz, 2H), 6.58-7.78 (m, 3H), 6.90-7.15 (m, 2H), 7.43 (d, J=9.3 Hz, 1H); ESI-MS (m/z) 399.09 (M+H)⁺.

### Example 1

### 4-[5-(3-Hydroxy-1-propynyl)-2-pyridyl]piperazino-2-trifluoromethylphenylmethanone

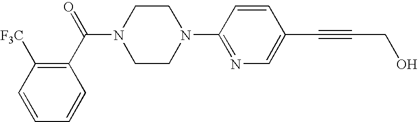

This product was prepared by Sonogashira coupling reaction of Intermediate 1 (1.1 g, 2.827 mmol) with prop-1-yn-1-ol (317 mg, 5.655 mmol) in the presence of PdCl₂(PPh₃)₂ (20 mg, 0.0284 mmol) and CuI (16 mg, 0.084 mmol) in triethylamine for 18 h under nitrogen. The crude product obtained after extractive work up using chloroform was purified by silica gel column chromatography using 30% EtOAc in chloroform to give 270 mg of the product as an off-white solid; IR (KBr) 3298, 2851, 2240, 1626, 1497, 1242, 1010, 769 cm⁻¹; ¹H NMR (300 MHz, CDCl₃) δ 1.81 (br s, 1H, D₂O exchangeable), 3.28 (br s, 2H), 3.54-3.68 (m, 4H), 3.88-3.95 (m, 2H), 4.48 (s, 2H), 6.58 (d, J=8.4 Hz, 1H), 7.36 (d, J=6.9 Hz, 1H), 7.52-7.62 (m, 3H), 7.74 (d, J=7.2 Hz, 1H), 8.26 (s, 1H); ESI-MS (m/z) 390.30 (M+H)⁺.

### Example 2

### 4-[5-(3-Hydroxy-1-propynyl)-2-pyridyl]piperazino-2,5-dichlorophenylmethanone

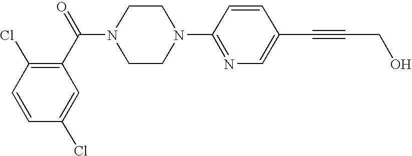

Prepared by Sonogashira coupling reaction of Intermediate 3 with prop-1-yn-1-ol to give the product as a white solid; IR (KBr) 3351, 2846, 2237, 1628, 1495, 1239, 1012, 822 cm⁻¹; ¹H NMR (300 MHz, CDCl₃) δ 1.71 (t, J=6.3 Hz, 1H, D₂O exchangeable), 3.30-3.39 (m, 2H), 3.60-3.69 (m, 4H), 3.85-3.96 (m, 2H), 4.49 (d, J=6.0 Hz, 2H), 6.59 (d, J=9.0 Hz, 1H), 7.31-7.35 (m, 3H), 7.53 (d, J=8.7 Hz, 1H), 8.27 (s, 1H); ESI-MS (m/z) 390.61 [100%, (M+H)⁺].

### Example 3

### 4-[6-(3-Hydroxy-1-propynyl)-3-pyridazinyl]piperazino-2-trifluoromethylphenyl-methanone

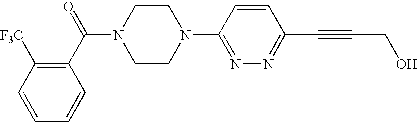

Prepared by Sonogashira coupling reaction of Intermediate 5 with prop-1-yn-1-ol to give the product as a white solid; IR (KBr) 3295, 2928, 1619, 1439, 1249, 1030, 773 cm⁻¹; ¹H NMR (300 MHz, DMSO-d₆) δ 3.21-3.82 (m, 8H), 3.33 (d, J=5.7 Hz, 2H), 5.44 (t, J=8.0 Hz, 1H), 7.24 (d, J=9.6 Hz, 1H), 7.59 (d, J=9.3 Hz, 1H), 7.56 (d, J=7.8 Hz, 1H), 7.65-7.86 (m, 3H); ESI-MS (m/z) 391.51 (M+H)⁺.

### Example 4

### 4-[5-(3-Hydroxy-3-methyl-1-butynyl)-2-pyrimidinyl]piperazino-2-trifluoromethylphenyl-methanone

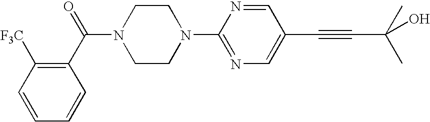

Prepared by Sonogashira coupling reaction of Intermediate 8 with 2-methylbut-3-yn-2-ol to give the product as a white solid; ¹H NMR (300 MHz, CDCl₃) δ 1.61 (s, 6H), 2.09 (br s, 1H), 3.24 (t, J=4.8 Hz, 2H), 3.78 (t, J=5.7 Hz, 2H), 3.83-4.00 (m, 4H), 7.36 (d, J=7.2 Hz, 1H), 7.55-7.64 (m, 2H), 7.74 (d, J=7.8 Hz, 1H), 8.34 (s, 2H); ESI-MS (m/z) 419.34 (M+H)⁺.

### Example 5

### 4-{5-[2-(1-Hydroxycyclopentyl)-1-ethynyl]-2-pyridyl}piperazino-2-trifluoromethyl phenylmethanone

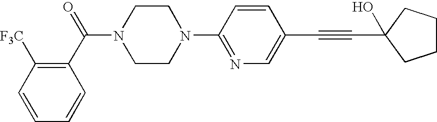

Prepared by Sonogashira coupling reaction of Intermediate 1 with 1-ethynylcyclopentanol to give the product as a white solid; ¹H NMR (300 MHz, DMSO-d₆) δ 1.67-1.85 (m, 8H), 3.16-3.70 (m, 8H), 5.27 (br s, 1H), 6.82 (d, J=8.7 Hz, 1H), 7.53-7.85 (m, 5H), 8.15 (s, 1H); ESI-MS (m/z) 444.32 (M+H)⁺.

### Example 6

### 4-[5-(3-Hydroxy-1-pentynyl)-2-pyridyl]piperazino-2-trifluoromethylphenylmethanone

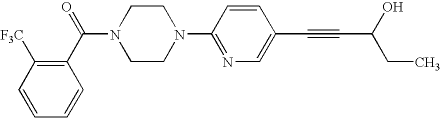

To a stirred and cooled (−70° C.) solution of Intermediate 2 (100 mg, 0.278 mmol) in dry THF (10 ml) was added 1.6 M n-butyllithium (0.37 ml, 0.40 mmol) in hexane. The mixture was stirred at the same temperature for 15 min and propionaldehyde (24 mg, 0.41 mmol) was added and stirred for another 20 min at −70° C. The mixture was quenched with water and extracted with ethyl acetate (2×20 ml). The combined organic extracts were washed with water (2×40 ml) and dried over Na₂SO₄. The crude product obtained after evaporation of the solvent was purified by silica gel column chromatography using 0.5% methanol in chloroform to give 50 mg of the product as a white solid; IR (KBr) 3412, 2919, 2214, 1639, 1496, 1291, 1009, 771 cm⁻¹; ¹H NMR (300 MHz, CDCl₃) δ 1.06 (t, J=7.8 Hz, 3H), 1.76-1.83 (m, 2H), 1.93 (d, J=5.4 Hz, 1H), 3.29 (br s, 2H), 3.54 (br s, 2H), 3.67 (br s, 2H), 3.88-3.95 (m, 2H), 4.54 (q, J=6.0 Hz, 1H), 6.59 (d, J=8.7 Hz, 1H), 7.36 (d, J=7.2 Hz, 1H), 7.51-7.57 (m, 3H), 7.73 (d, J=7.8 Hz, 1H), 8.25 (s, 1H); ESI-MS (m/z) 418.39 (M+H)⁺.

### Example 7

### 4-[5-{3-Hydroxy-3-(1-adamantyl)-1-propynyl}-2-pyridyl]piperazino-2-trifluoromethyl-phenylmethanone

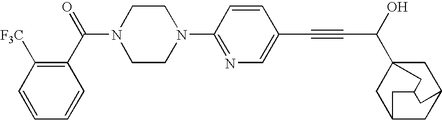

This compound was prepared in the same manner as described in Example 6 from Intermediate 2 (200 mg, 0.557 mmol) and 1-admantanecarboxaldehyde (91 mg, 0.557 mmol) using 1.6 M n-butyllithium (0.535 ml, 0.835 mmol) in hexane as the base to give 140 mg of the product as a white solid; IR (KBr) 3434, 2904, 2218, 1645, 1495, 1316 1009 cm⁻¹; ¹H NMR (300 MHz, CDCl₃) δ 1.56-1.80 (m, 14H), 2.03 (br s, 2H), 3.28 (br s, 2H), 3.53 (br s, 2H), 3.66 (br s, 2H), 3.80-4.00 (m, 2H), 4.06 (s, 1H), 6.57 (d, J=9.0 Hz, 1H), 7.36 (d, J=6.6 Hz, 1H), 7.52-7.75 (m, 4H), 8.26 (s, 1H); ESI-MS (m/z) 524.79 (M+H)⁺.

### Example 8

### 4-[5-(3-Hydroxy-3-phenyl-1-propynyl}-2-pyridyl}piperazino-2-trifluoromethylphenyl-methanone

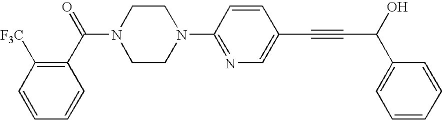

This compound was prepared in the same manner as described in Example 6 from Intermediate 2 (250 mg, 0.696 mmol) and benzaldehyde (81.3 mg, 0.765 mmol) using 1.6 M n-butyllithium (0.653 ml, 1.031 mmol) in hexane as the base to give 90 mg of the product as a white solid; IR (KBr) 3397, 2855, 2182, 1638, 1496, 1241, 1009, 773 cm⁻¹; ¹H NMR (300 MHz, CDCl₃) δ 2.53 (br s, 1H), 3.28 (br s, 2H), 3.52 (br s, 2H), 3.66 (br s, 2H), 3.82-4.01 (m, 2H), 5.68 (s, 1H), 6.57 (d, J=9.0 Hz, 1H), 7.34-7.40 (m, 4H), 7.55-7.61 (m, 5H), 7.73 (d, J=7.8 Hz, 1H), 8.30 (s, 1H); ESI-MS (m/z) 466.51 (M+H)⁺.

### Example 9

### 4-[5-(3-Cyclopentyloxy-1-propynyl)-2-pyridyl]piperazino-2-trifluoromethylphenyl-methanone

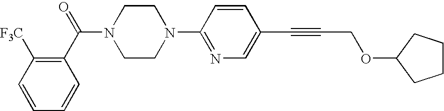

Prepared by Sonogashira coupling reaction of Intermediate 1 with 1-(2-propynyloxy)cyclopentane (161 mg, 1.301 mmol) to give the product as an off-white solid; IR (KBr) 2954, 2226, 1648, 1600, 1493, 1241, 1009, 771 cm⁻¹; ¹H NMR (300 MHz, CDCl₃) δ 1.45-1.80 (m, 8H), 3.25 (br s, 2H), 3.47 (br s, 2H), 3.65 (br s, 2H), 3.82-4.14 (m, 2H), 4.18 (br s, 1H), 4.25 (s, 2H), 6.57 (d, J=8.7 Hz, 1H), 7.36 (d, J=7.2 Hz, 1H), 7.52-7.65 (m, 3H), 7.74 (d, J=7.8 Hz, 1H), 8.27 (s, 1H); ESI-MS (m/z) 458.52 (M+H)⁺.

### Example 10

### 4-{4-[3-(4-tert-Butylphenoxy)-1-propynyl]-2-pyridyl}piperazino-2-trifluoromethyl-phenylmethanone

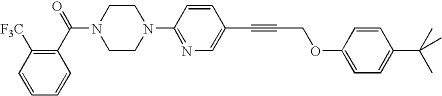

Prepared by Sonogashira coupling reaction of Intermediate 1 with 1-(tert-butyl)-4-(2-propynyloxy)benzene to give the product as a white solid; IR (KBr) 2955, 2227, 1638, 1493, 1318, 1012 cm⁻¹; ¹H NMR (300 MHz, CDCl₃) δ 1.30 (s, 9H), 3.28 (br s, 2H), 3.54 (br s, 2H), 3.68 (br s, 2H), 3.80-4.00 (m, 2H), 4.88 (s, 2H), 6.57 (d, J=8.4 Hz, 1H), 6.95 (d, J=9.0 Hz, 2H), 7.31-7.40 (m, 3H), 7.52-7.62 (m, 3H), 7.74 (d, J=6.9 Hz, 1H), 8.26 (s, 1H); ESI-MS (m/z) 522.75 (M+H)⁺.

### Example 11

### 4-[5-(3-(4-Fluorophenoxy)-1-propynyl)-2-pyridyl]piperazino-2-trifluoromethylphenyl-methanone

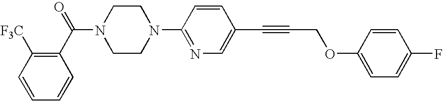

Step 1: tert-Butyl 4-{5-[3-(4-fluorophenoxy)-1-propynyl]-2-pyridyl}-1-piperazine carboxylate: tert-Butyl 4-pyridin-2-ylpiperazine-1-carboxylate (900 mg, 2.313 mmol) was coupled with 1-fluoro-4-(2-propynyloxy)benzene (694 mg, 4.627 mmol) under Sonogashira reaction conditions using catalytic amounts of PdCl₂(PPh₃)₂ (32.5 mg, 0.046 mmol) and CuI (13.2 mg, 0.069 mmol) in TEA (10 ml) to give 610 mg of the product as an off-white solid; IR (KBr) 3436, 2983, 2223, 1693, 1505, 1239, 1013, 831 cm⁻¹; ¹H NMR (300 MHz, CDCl₃) δ 1.48 (s, 9H), 3.54 (d, J=5.7 Hz, 8H), 4.86 (s, 2H), 6.54 (d, J=8.7 Hz, 1H), 6.97 (br s, 4H), 7.50 (d, J=6.3 Hz, 1H), 8.25 (s, 1H); ESI-MS (m/z) 412.37 (M+H)⁺.

Step 2: 1-{5-[3-(4-Fluorophenoxy)-1-propynyl]-2-pyridyl}piperazine hydrochloride: Step 1 intermediate (600 mg, 1.459 mmol) was treated with 15% HCl in EtOAc (12 ml) and stirred at room temperature for 30 min. The mixture was evaporated to dryness to give 454 mg of the product as a white solid, which was used as such for the next step.

Step 3: 4-[5-(3-(4-Fluorophenoxy)-1-propynyl)-2-pyridyl]piperazino-2-trifluoromethyl phenylmethanone: To a stirred suspension of Step 2 intermediate (300 mg, 0.729 mmol) in dichloromethane (20 ml) was added 2-(trifluoromethyl)benzoic acid (167 mg, 0.875 mmol), EDCI (148 mg, 1.094 mmol), HOBT (112 mg, 0.729 mmol) followed by triethylamine (185 mg, 1.824 mmol). The homogeneous solution was stirred at room temperature for 18 h under nitrogen atmosphere. Extractive work-up with dichloromethane followed by purification by silica gel column chromatography using 30% EtOAc in chloroform gave 183 mg of the product as a white solid; IR (KBr) 3001, 2915, 2229, 1645, 1504, 1242, 1008, 829 cm⁻¹; ¹H NMR (300 MHz, CDCl₃) δ 3.27-3.94 (m, 8H), 4.86 (s, 2H), 6.56 (d, J=8.7 Hz, 1H), 6.97-7.00 (m, 4H), 7.35 (d, J=6.9 Hz, 1H), 7.50-7.65 (m, 3H), 7.73 (d, J=7.5 Hz, 1H), 8.25 (s, 1H); ESI-MS (m/z) 484.29 (M+H)⁺.

### Example 12

### 6-(3-{6-[4-(2-Trifluoromethylbenzoyl)piperazino]-3-pyridyl}-2-propynyloxy)nicotino-nitrile

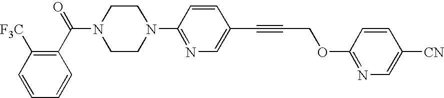

Prepared by Sonogashira coupling reaction of Intermediate 1 with 6-(2-propynyloxy)nicotinonitrile to give the product as a white solid; IR(KBr) 2862, 2229, 1642, 1488, 1317, 1242 cm⁻¹; ¹H NMR (300 MHz, CDCl₃) δ 3.28 (br s, 2H), 3.55 (br s, 2H), 3.69 (br s, 2H), 3.88-3.95 (m, 2H), 5.25 (s, 2H), 6.56 (d, J=9.0 Hz, 1H), 6.91 (d, J=7.8 Hz, 1H), 7.35 (d, J=7.2 Hz, 1H), 7.55-7.26 (m, 3H), 7.73 (d, J=7.2 Hz, 1H), 7.83 (d, J=9.0 Hz, 1H), 8.27 (s, 1H), 8.52 (s, 1H); ESI-MS (m/z) 492.45 (M+H)⁺.

### Example 13

### 4-{5-[3-(4-Hydroxyphenoxy)-1-propynyl]-2-pyridyl}piperazino-2-trifluoromethyl-methanone

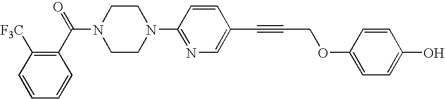

Step 1: 4-(3-{6-[4-(2-Trifluoromethylbenzoyl)piperazino]-3-pyridyl}-2-propynyloxy)phenyl acetate: Prepared by Mitsunobu coupling reaction of Example 1 (500 mg, 1.285 mmol) with 4-hydroxyphenyl acetate (196 mg, 1.285 mmol) in the presence of triphenylphosphine (506 mg, 1.927 mmol) and DEAD (291 mg, 1.6709 mmol) in THF (10 ml) for 18 h at 65-70° C. The reaction mixture was concentrated in vacuum and the residue obtained was purified by silica gel column chromatography using 20% acetone in petroleum ether to give 400 mg of the product as an off-white solid; IR (KBr) 2904, 2227, 1754, 1598, 1318, 1192, 1008, 767 cm⁻¹; ¹H NMR (300 MHz, CDCl₃) δ 2.28 (s, 3H), 3.28 (br s, 2H), 3.54 (br s, 2H), 3.60-3.78 (m, 2H), 3.82-4.14 (m, 2H), 4.88 (s, 2H), 6.57 (d, J=9.0 Hz, 1H), 7.01 (s, 4H), 7.35 (d, J=7.2 Hz, 1H), 7.51-7.70 (m, 3H), 7.73 (d, J=7.5 Hz, 1H), 8.25 (s, 1H); ESI-MS (m/z) 524.76 (M+H)⁺.

Step 2: Deacetylation of Step 1 intermediate under basic conditions gave the product as an off-white solid; IR (KBr) 3411, 2229, 1627, 1508, 1316, 1242, 1009, 771 cm⁻¹; ¹H NMR (300 MHz, CDCl₃) δ 3.28 (br s, 2H), 3.54 (br s, 2H), 3.68 (m, 2H), 3.84-4.01 (m, 2H), 4.83 (s, 2H), 4.94 (s, 1H), 6.56 (d, J=9.0 Hz, 1H), 6.77 (d, J=8.7 Hz, 2H), 6.91 (d, J=8.7 Hz, 2H), 7.35 (d, J=7.2 Hz, 1H), 7.51-7.62 (m, 3H), 7.74 (d, J=6.9 Hz, 1H), 8.25 (s, 1H); ESI-MS (m/z) 482.58 (M+H)⁺.

### Example 14

### 1-{5-[3-(4-Fluorophenoxy)prop-1-yn-1-yl]-2-pyridyl}-4-(5-trifluoromethylpyridin-2-yl)piperazine

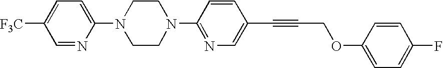

Step 1: tert-Butyl 4-{5-[3-(4-fluorophenoxy)prop-1-yn-1-yl]pyridin-2-yl}piperazine-1-carboxylate: To a stirred solution of tert-Butyl 4-(5-iodopyridin-2-yl)piperazine-1-carboxylate (900 mg, 2.313 mmol) in triethylamine (15 ml) was added 1-fluoro-4-(prop-2-yn-1-yloxy)benzene (694 mg, 4.627 mmol), (PPh₃)₂PdCl₂ (325 mg, 0.462 mmol) and CuI (132 mg, 0.694 mmol) at room temperature under nitrogen atmosphere. The reaction mixture was stirred at the same temperature for 6 days. The mixture was diluted with water (50 ml) and extracted with EtOAc (2×50 ml). The organic layer was washed with water (50 ml) and dried over anhydrous Na₂SO₄. The solvent was evaporated and the crude product was purified by silica gel column chromatography using 20% EtOAc in petroleum ether to give 610 mg of the product as an off-white solid; IR (KBr) 2983, 2857, 2223, 1693, 1493, 1239, 1013, 831 cm⁻¹; ¹H NMR (300 MHz, CDCl₃) δ 1.48 (s, 9H), 3.55 (d, J=6.0 Hz, 9H), 4.87 (s, 2H), 6.54 (d, J=8.4 Hz, 1H), 6.97 (s, 4H), 7.50 (d, J=8.7 Hz, 1H), 8.25 (s, 1H); ESI-MS (m/z) 412.39 (M+H)⁺.

Step 2: 1-{5-[3-(4-Fluorophenoxy)prop-1-yn-1-yl]pyridin-2-yl}piperazine: Step 1 intermediate (320 mg, 1.028 mmol) was deprotected using TFA (5 ml) to give 420 mg of the product as an off-white solid which was used as such for the next step.

Step 3: 1-{5-[3-(4-Fluorophenoxy)prop-1-yn-1-yl]pyridin-2-yl}-4-(5-trifluoromethylpyridin-2-yl)piperazine: To a stirred solution of Step 2 intermediate (320 mg, 1.028 mmol) in dry toluene (20 ml) was added 2-chloro-5-(trifluoromethyl)pyridine (224 mg, 1.234 mmol), potassium-tert-butoxide (210 mg, 1.543 mmol), (2-biphenyl)di-tert-butylphosphine (10 mg) and Pd(II) acetate (10 mg) at room temperature. The reaction mixture was further stirred at 115° C. overnight. The mixture was cooled, diluted with water (50 ml) and extracted with ethyl acetate (2×50 ml). The combined organic extracts were washed with water (2×100 ml) and dried over anhydrous Na₂SO₄. The crude product obtained after evaporation of the solvent was purified by silica gel column chromatography using 20% ethyl acetate in petroleum ether to give 30 mg of the product as an off-white solid; IR (KBr) 2923, 2855, 2222, 1611, 1508, 1493, 1244, 814 cm⁻¹; ¹H NMR (300 MHz, CDCl₃) δ 3.77 (s, 9H), 4.87 (s, 2H), 6.56-6.67 (dd, J=2.5, 6.8 Hz, 2H), 6.97-7.03 (m, 4H), 7.53 (d, J=6.0 Hz, 1H), 7.66 (d, J=6.0 Hz, 1H), 8.27 (s, 1H), 8.14 (s, 1H); ESI-MS (m/z) 457.50 (M+H)⁺.

### Example 15

### N1-(3-{6-[4-(2-Trifluoromethylbenzoyl)piperazino]-3-pyridyl}-2-propynyl)acetamide

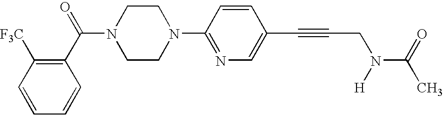

This compound was prepared as described in the general procedure, from Intermediate 1 (200 mg, 0.867 mmol) and N-propynylacetamide (168 mg, 1.735 mmol) in the presence of PdCl₂(PPh₃)₂ (61 mg, 0.0867 mmol) and CuI (50 mg, 0.2602 mmol) in TEA (10 ml) to give 100 mg of the product as an off-white solid; IR (KBr) 3287, 2235, 1638, 1497, 1242 cm⁻¹; ¹H NMR (300 MHz, DMSO-d₆) δ 1.84 (s, 3H), 3.14-3.50 (m, 4H), 3.67-3.77 (m, 4H), 4.08 (d, J=5.1 Hz, 2H), 6.83 (d, J=8.7 Hz, 1H), 7.53-7.60 (m, 2H), 7.76 (t, J=7.5 Hz, 1H), 7.78 (t, J=7.2 Hz, 1H), 7.88 (d, J=7.8 Hz, 1H), 8.18 (d, J=2.1 Hz, 1H), 8.37 (br s, 1H); ESI-MS (m/z) 431.03 (M+H)⁺.

### Example 16

### N1-(3-{6-[4-(2-Trifluoromethylbenzoyl)piperazino]-3-pyridyl}-2-propynyl)-1-butane sulfonamide

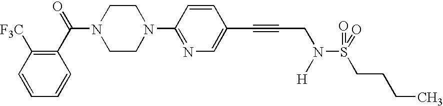

Prepared by Sonogashira coupling reaction of Intermediate 1 with N1-(2-propynyl)-1-butanesulfonamide in triethylamine to give 158 mg of the product as an off-white solid; IR (KBr) 2930, 2217, 1638, 1497, 1242, 1008, 771 cm⁻¹; ¹H NMR (300 MHz, CDCl₃) δ 0.91 (t, J=6.0 Hz, 3H), 1.40-1.47 (m, 2H), 1.79-1.90 (m, 2H), 3.17 (t, J=7.5 Hz, 2H), 3.28 (br s, 2H), 3.55 (br s, 2H), 3.69 (br s, 2H), 3.85-4.0 (m, 2H), 4.17 (d. J=6.3 Hz, 2H), 4.52 (br s, 1H), 6.58 (d. J=9 Hz, 1H), 7.36 (d, J=7.2 Hz, 1H), 7.47-7.77 (m, 4H), 8.21 (s, 1H); ESI-MS (m/z) 507.41 (M−H)⁻.

### Example 17

### 4-[5-(1-Pentynyl)-2-pyridyl]piperazino-2-trifluoromethylphenylmethanone

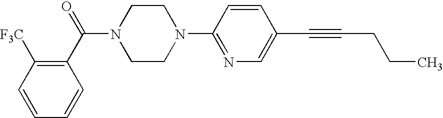

Prepared by Sonogashira coupling reaction of Intermediate 1 with 1-pentyne to give the product as a white solid; IR (KBr) 2898, 1639, 1491, 1315, 1169, 1008 cm⁻¹; ¹H NMR (300 MHz, DMSO-d₆) δ 1.03 (t, J=7.2 Hz, 3H), 1.55-1.65 (m, 2H), 2.37 (t, J=6.6 Hz, 2H), 3.28 (br s, 2H), 3.51 (br s, 2H), 3.64 (br s, 2H), 3.89-3.98 (m, 2H), 6.56 (d, J=8.7 Hz, 1H), 7.35 (d, J=7.2 Hz, 1H), 7.49-7.62 (m, 3H), 7.73 (d, J=7.5 Hz, 1H), 8.22 (s, 1H); ESI-MS (m/z) 402.62 (M+H)⁺.

### Example 18

### 4-[5-(3,3-Dimethyl-1-butynyl)-2-pyridyl]piperazino-2-trifluoromethylphenylmethanone

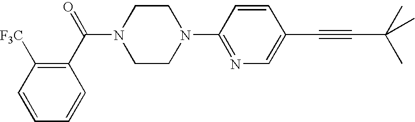

Prepared by Sonogashira coupling reaction of Intermediate 1 with 3,3-dimethyl-1-butyne to give product as a white solid; IR (KBr) 2968, 1647, 1493, 1240, 1012 cm⁻¹; ¹H NMR (300 MHz, CDCl₃) δ 1.30 (s, 9H), 3.26-3.29 (m, 2H), 3.49-3.55 (m, 2H), 3.63-3.66 (m, 2H), 3.85-3.95 (m, 2H), 6.56 (d, J=9.0 Hz, 1H), 7.35 (d, J=7.2 Hz, 1H), 7.47-7.65 (m, 3H), 7.76 (d, J=7.8 Hz, 1H), 8.20 (d, J=1.8 Hz, 1H); ESI-MS (m/z) 416.68 (M+H)⁺.

### Example 19

### 2,5-Dichlorophenyl-4-[5-(3,3-dimethyl-1-butynyl)-2-pyridyl]piperazinomethanone

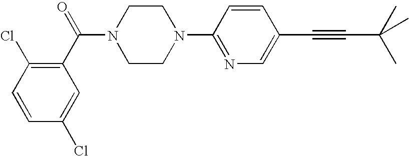

Prepared by Sonogashira coupling reaction of Intermediate 3 with 3,3-dimethyl-1-butyne to give the product as a white solid; IR (KBr) 2968, 1647, 1493, 1240, 1012, 814 cm⁻¹; ¹H NMR (300 MHz, CDCl₃) δ 1.30 (s, 9H), 3.30-34 (m, 2H), 3.58-3.67 (m, 4H), 3.87-3.99 (m, 2H), 6.55 (d, J=9.0 Hz, 1H), 7.31-7.38 (m, 3H), 7.50 (d, J=8.4 Hz, 1H), 8.21 (s, 1H); ESI-MS (m/z) 418.63 [100%, (M+H)⁺].

### Example 20

### 4-[5-(2-Phenyl-1-ethynyl)-2-pyridyl]piperazino-2-trifluoromethylphenylmethanone

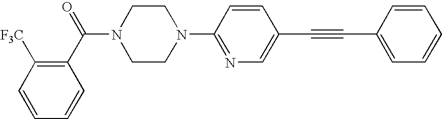

Prepared by Sonogashira coupling reaction of Intermediate 1 with phenyl acetylene to give the product as a white solid; IR (KBr) 2898, 2210, 1644, 1502, 1242, 1010, 769 cm⁻¹; ¹H NMR (300 MHz, CDCl₃) δ 3.28-3.31 (m, 2H), 3.54-3.58 (m, 2H), 3.68-3.76 (m, 2H), 3.86-4.00 (m, 2H), 6.62 (d, J=8.7 Hz, 1H), 7.32-7.38 (m, 4H), 7.49-7.64 (m, 5H), 7.73 (d, J=8.1 Hz, 1H), 8.35 (d, J=2.1 Hz, 1H); ESI-MS (m/z) 436.29 (M+H)⁺.

### Example 21

### 2,5-Dichlorophenyl-4-[5-(2-phenyl-1-ethynyl)-2-pyridyl]piperazinomethanone

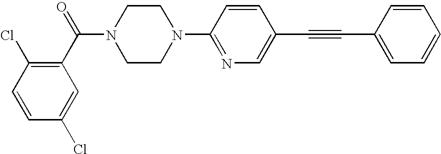

Prepared by Sonogashira coupling reaction of Intermediate 3 with phenyl acetylene to give the product as a white solid; IR (KBr) 2914, 2212, 1645, 1503, 1240, 1006, 807 cm⁻¹; ¹H NMR (300 MHz, CDCl₃) δ 3.34-3.42 (m, 2H), 3.59-3.70 (m, 4H), 3.80-4.01 (m, 2H), 6.63 (d, J=8.7 Hz, 1H), 7.32-7.36 (m, 6H), 7.49-7.52 (m, 2H), 7.63 (dd, J=6.6, 2.1 Hz, 1H), 8.36 (d, J=12.5 Hz, 1H); ESI-MS (m/z) 436.46 [100%, (M+H)⁺].

### Example 22

### 4-(2-{4-[4-(2-Trifluoromethylbenzoyl)piperazino]pyridinyl-1-ethynyl)phenyl acetate

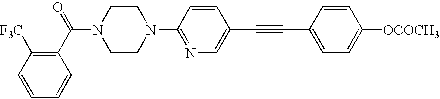

Prepared by Sonogashira coupling reaction of Intermediate 2 with 4-iodophenyl acetate as a white solid; IR (KBr) 2923, 2207, 1767, 1507, 1247, 1010, 775 cm⁻¹; ¹H NMR (300 MHz, CDCl₃) δ 3.29 (br s, 2H), 3.55 (br s, 2H), 3.69 (br s, 2H), 3.82 (s, 3H), 3.90-3.40 (m, 2H), 6.61 (d, J=9 Hz, 1H), 6.85-6.88 (m, 2H), 7.37-7.45 (m, 3H), 7.55-7.62 (m, 3H), 7.73 (d, J=7.2 Hz, 1H), 8.33 (s, 1H); ESI-MS (m/z) 494.53 (M+H)⁺.

### Example 23

### 4-{5-[2-(4-Hydroxyphenyl)-1-ethynyl]-2-pyridyl}piperazino-2-trifluoromethylphenyl methanone

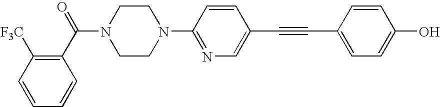

Deacetylation of Example 22 gave the product as an off-white solid; IR (KBr) 3248, 2925, 1607, 1316, 1245, 1009, 771 cm⁻¹; ¹H NMR (300 MHz, CDCl₃) δ 3.30 (br s, 2H), 3.55 (br s, 2H), 3.69 (br s, 2H), 3.89-4.00 (m, 2H), 5.21 (br s, 1H), 6.61 (d, J=8.4 Hz, 1H), 6.79 (d, J=8.7 Hz, 2H), 7.37-7.40 (m, 3H), 7.55-7.65 (m, 3H), 7.74 (d, J=8.1 Hz, 1H), 8.32 (s, 1H); ESI-MS (m/z) 450.41 (M−H)⁻.

### Example 24

### 1-{5-[(3-Fluoro-4-hydroxyphenyl)ethynyl]-2-pyridyl}piperazin-4-yl-(2-trifluoromethyl phenyl)methanone

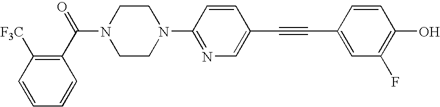

Step 1: 2-Fluoro-4-[2-{4-(2-trifluoromethylbenzoyl)piperiazin}pyridinyl-1-ethynyl]-phenyl acetate: Prepared by Sonogashira coupling reaction of Intermediate 1 with 4-ethynyl-2-fluorophenyl acetate to give the product as a light yellow solid; ¹H NMR (300 MHz, CDCl₃) δ 2.33 (s, 3H), 3.28 (br s, 2H), 3.56 (br s, 2H), 3.64-3.76 (m, 2H), 3.84-4.04 (m, 2H), 6.59 (d, J=8.7 Hz, 1H), 7.07 (t, J=8.1 Hz, 1H), 7.34 (t, J=7.8 Hz, 2H), 7.49-7.64 (m, 4H), 7.71 (d, J=8.4 Hz, 1H), 8.30 (s, 1H); ESI-MS (m/z) 512.51 (M+H)⁺.

Step 2: Deacetylation of Step 1 intermediate gave the product as an off-white solid; IR (KBr) 3152, 2952, 1597, 1518, 1317, 1287, 1010, 769 cm⁻¹; ¹H NMR (300 MHz, CDCl₃) δ 3.28 (br s, 2H), 3.55 (br s, 2H), 3.68 (br s, 2H), 3.80-4.00 (m, 2H), 5.73 (br s, 1H, D₂O exchangeable), 6.59 (br s, 1H), 6.82-7.00 (m, 2H), 7.10-7.30 (m, 3H), 7.34 (br s, 1H), 7.56 (br s, 1H), 7.70 (br s, 1H), 8.29 (s, 1H); ESI-MS (m/z) 470.39 (M+H)⁺.

### Example 25

### 4-{5-[2-(3-Hydroxyphenyl)-1-ethynyl]-2-pyridyl}piperazino-2-trifluoromethylphenyl methanone

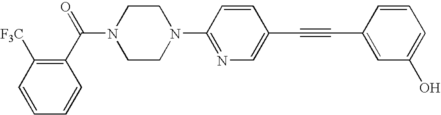

Step 1: 3-(2-{4-[4-(2-Trifluoromethylbenzoyl)piperazino]phenyl-1-ethynyl)phenyl acetate: Prepared by coupling reaction of Intermediate 2 with 3-iodophenyl acetate to give the product as an off-white solid; IR (KBr) 2923, 2207, 1767, 1638, 1507, 1201, 1010, 775 cm⁻¹; ¹H NMR (300 MHz, DMSO-d₆) δ 2.27 (s, 3H), 3.17-3.28 (m, 2H), 3.41-3.80 (m, 6H), 6.99 (d, J=8.7 Hz, 1H), 7.15 (d, J=7.8 Hz, 1H), 7.30 (s, 1H), 7.38-7.47 (m, 2H), 7.55 (d, J=7.5 Hz, 1H), 7.68-7.80 (m, 3H), 7.85 (d, J=7.8 Hz, 1H), 8.32 (d, J=1.8 Hz, 1H); ESI-MS (m/z) 494.53 (M+H)⁺.

Step 2: Deacetylation of Step 1 intermediate gave the product as a white solid; IR (KBr) 3227, 2908, 2206, 1627, 1588, 1244, 771 cm⁻¹; ¹H NMR (300 MHz, DMSO-d₆) δ 3.17-3.58 (m, 4H), 3.60-3.80 (m, 4H), 6.78 (d, J=8.1 Hz, 1H), 6.86 (s, 1H), 6.89-6.93 (m, 2H), 7.19 (t, J=7.5 Hz, 1H), 7.55 (d, J=7.5 Hz, 1H), 7.68-7.86 (m, 4H), 8.30 (s, 1H), 9.68 (s, 1H); ESI-MS (m/z) 450.81 (M−H)⁻.

### Example 26

### Ethyl-2-[3-(2-{6-[4-(2-trifluoromethylbenzoyl)piperazino]-3-pyridyl}-1-ethynyl)-phenoxy]acetate

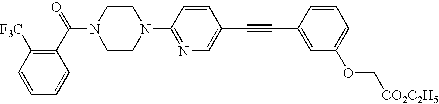

To a stirred suspension of Example 25 (250 mg, 0.55 mmol) and K₂CO₃ (114 mg, 0.71 mmol) in DMF (10 ml) was added ethyl bromoacetate (120 mg, 0.71 mmol) and the mixture was stirred at room temperature for 18 h under nitrogen atmosphere. The mixture was diluted with water (20 ml) and extracted with ethyl acetate (2×20 ml). The combined organic layer was washed with water (3×40 ml) and dried over Na₂SO₄. The crude product obtained after evaporation of the solvent was purified by recrystallization from ethyl acetate to give 206 mg of the product as an off-white solid; IR (KBr) 2905, 2201, 1753, 1638, 1499, 1318, 1131 cm⁻¹; ¹H NMR (300 MHz, CDCl₃) δ 1.30 (t, J=7.5 Hz, 3H), 3.29 (br s, 2H), 3.56 (br s, 2H), 3.69 (br s, 2H), 3.80-3.90 (m, 2H), 4.27 (q, J=7.2 Hz, 2H), 4.62 (s, 2H), 6.60 (d, J=8.7 Hz, 1H), 6.89 (d, J=9.3 Hz, 1H), 7.00 (s, 1H), 7.11-7.13 (m, 1H), 7.21-7.24 (m, 1H), 7.34 (d, J=7.8 Hz, 1H), 7.53-7.66 (m, 3H), 7.72 (d, J=7.8 Hz, 1H), 8.31 (s, 1H).

### Example 27

### 2-[3-(2-{6-[4-(2-Trifluoromethylbenzoyl)piperazino]-3-pyridyl}-1-ethynyl)phenoxy]-acetic acid

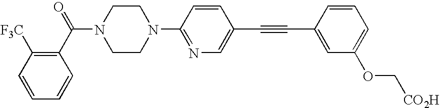

To a stirred solution of Example 26 (120 mg) in ethanol (5 ml) was added 1N NaOH solution (5 ml) and the mixture was stirred at room temperature for 2 h under nitrogen atmosphere. The mixture was diluted with water (10 ml) and the pH of the solution was adjusted to 4 with acetic acid to result a precipitate. The product was collected by filtration, washed with diethyl ether (5 ml) and dried to give 70 mg of the product as a off white solid; IR (KBr) 3437, 2920, 2205, 1743, 1643, 1498, 1316, 1174, 1008 cm⁻¹; ¹H NMR (300 MHz, CD₃OD) δ 3.29 (br s, 2H), 3.57 (br s, 2H), 3.75 (br s, 2H), 3.80-3.90 (m, 2H), 4.64 (s, 2H), 6.83 (d, J=8.7 Hz, 1H), 6.92 (d, J=8.4 Hz, 1H), 7.02 (s, 1H), 7.07 (d, J=7.8 Hz, 1H), 7.23-7.28 (m, 1H), 7.49 (d, J=7.8 Hz, 1H), 7.63-7.73 (m, 3H), 7.80 (d, J=7.2 Hz, 1H), 8.25 (s, 1H); ESI-MS (m/z) 510.89 (M+H)⁺.

### Example 28

### 2,5-Dichlorophenyl-4-{5-[2-(3-hydroxy-1-pentynyl}-1-ethynyl]-2-pyridyl}piperazino methanone

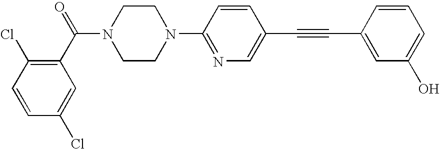

Step 1: 3-(2-[6-[4-(2,5-Dichlorobeznoyl)piperazino]-3-pyridyl]-1-ethynyl)phenyl acetate: Prepared by Sonogashira coupling reaction of Intermediate 4 with 3-iodophenyl acetate to give the product as an off-white solid; ¹H NMR (300 MHz, CDCl₃) δ 2.31 (s, 3H), 3.24-3.50 (m, 2H), 3.60-3.80 (m, 4H), 3.84-4.05 (m, 2H), 6.62 (d, J=8.4 Hz, 1H), 7.00-7.10 (m, 1H), 7.30-7.40 (m, 6H), 7.61 (d, J=7.5 Hz, 1H), 8.34 (s, 1H).

Step 2: Deacetylation of Step 1 intermediate gave the product as a white solid; IR (KBr) 3434, 2925, 2203, 1624, 1594, 1499, 1241, 1012 cm⁻¹; ¹H NMR (300 MHz, CDCl₃) δ 3.30-3.45 (m, 3H), 3.60-3.70 (m, 4H), 3.86-4.01 (m, 2H), 6.64 (d, J=9.0 Hz, 1H), 6.81 (dd, J=2.1, 6.0 Hz, 1H), 6.96 (s, 1H), 7.02 (d, J=7.8 Hz, 1H), 7.18 (t, J=8.1 Hz, 1H), 7.30-7.40 (m, 3H), 7.63 (dd, J=6.6, 2.1 Hz, 1H), 8.33 (d, J=2.1 Hz, 1H); ESI-MS (m/z) 452.75 [100%, (M+H)⁺].

### Example 29

### 2-(2-{4-[4-(2-Trifluoromethylbenzoyl)piperazino]pyridinyl-1-ethynyl)phenyl acetate

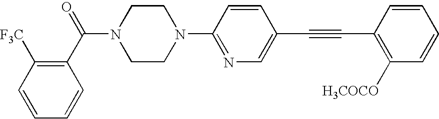

Prepared by Sonogashira coupling reaction of Intermediate 2 with 2-iodophenyl acetate to give the product as a white solid; IR (KBr) 2923, 2213, 1769, 1646, 1594, 1316, 1172, 1008 cm⁻¹; ¹H NMR (300 MHz, CDCl₃) δ 2.35 (s, 3H), 3.28-3.31 (m, 2H), 3.55-3.60 (m, 2H), 3.69-3.75 (m, 2H), 3.80-4.00 (m, 2H), 6.62 (d, J=8.7 Hz, 1H), 7.11 (d, J=8.1 Hz, 1H), 7.26-7.30 (m, 1H), 7.32-7.38 (m, 2H), 7.53-7.63 (m, 4H), 7.74 (d, J=7.2 Hz, 1H), 8.31 (s, 1H); ESI-MS (m/z) 494.73 (M+H)⁺.

### Example 30

### 4-{5-[2-(4-Methoxyphenyl)-1-ethynyl]-2-pyridyl}piperazino-2-trifluoromethylphenyl methanone

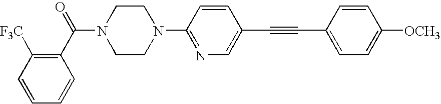

Prepared by Sonogashira coupling reaction of Intermediate 2 with 4-iodoanisole to give the product as an off-white solid; IR (KBr) 3446, 2916, 2207, 1629, 1244, 1012, 775 cm⁻¹; ¹H NMR (300 MHz, CDCl₃) δ 3.29 (br s, 2H), 3.55 (br s, 2H), 3.69 (br s, 2H), 3.82 (s, 3H), 3.89-3.99 (m, 2H), 6.61 (d, J=8.7 Hz, 1H), 6.87 (d, J=8.4 Hz, 2H), 7.36 (d, J=7.5 Hz, 1H), 7.44 (d, J=8.4 Hz, 2H), 7.52-7.65 (m, 3H), 7.74 (d, J=7.8 Hz, 1H), 8.33 (s, 1H); ESI-MS (m/z) 466.57 (M+H)⁺.

### Example 31

### 2,5-Dichlorophenyl-4-{5-[2-(3-methoxyphenyl)-1-ethynyl]-2-pyridyl}piperazino-methanone

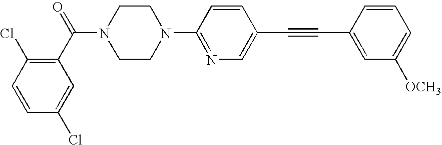

Prepared by Sonogashira coupling reaction of Intermediate 4 with 3-iodoanisole to give the product as an off-white solid; IR (KBr) 2922, 2201, 1647, 1589, 1499, 1241, 1011 cm⁻¹; ¹H NMR (300 MHz, CDCl₃) δ 3.35-3.40 (m, 2H), 3.60-3.72 (m, 4H), 3.82 (s, 3H), 3.84-4.01 (m, 2H), 3.63 (d, J=8.7 Hz, 1H), 6.87 (dd, J=6.3, 3.0 Hz, 1H), 7.03 (s, 1H), 7.11 (d, J=7.2 Hz, 1H), 7.25-7.39 (m, 4H), 7.63 (dd, J=6.6, 2.1 Hz, 1H), 8.36 (d, J=1.8 Hz, 1H); ESI-MS (m/z) 467.30 [100%, (M+H)⁺].

### Example 32

### Methyl-4-(2-{6-[4-(2-trifluoromethylbenzoyl)piperazino]-2-pyridyl-1-ethynyl)benzoate

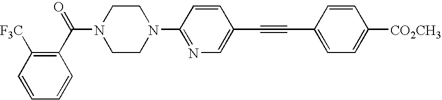

Prepared by Sonogashira coupling reaction of Intermediate 2 with methyl 4-iodobenzoate to give the product as an off-white solid; IR (KBr) 2859, 2210, 1714, 1649, 1596, 1316, 1009 cm⁻¹; ¹H NMR (300 MHz, CDCl₃) δ 3.31 (br s, 2H), 3.58 (br s, 2H), 3.72 (br s, 2H), 3.92 (s, 3H), 3.88-4.08 (m, 2H), 6.63 (d, J=9.3 Hz, 1H), 7.37 (d, J=7.8 Hz, 1H), 7.54-7.68 (m, 5H), 7.75 (d, J=7.5 Hz, 1H), 8.01 (d, J=8.1 Hz, 2H), 8.36 (s, 1H); ESI-MS (m/z) 494.28 (M+H)⁺.

### Example 33

### 4-{5-[2-(3-Hydroxymethylphenyl)-1-ethynyl]-2-pyridyl}piperazino-2-trifluoromethyl-phenylmethanone

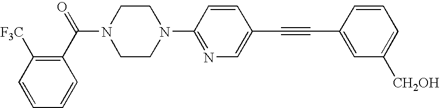

Prepared by Sonogashira coupling reaction of Intermediate 2 with 3-iodophenylmethanol to give the product as an off-white solid; IR (KBr) 3409, 2853, 2202, 1628, 1595, 1316, 1115 cm⁻¹; ¹H NMR (300 MHz, CDCl₃) δ 1.83 (s, 1H, D₂O exchangeable) 3.29 (br s, 2H), 3.56 (br s, 2H), 3.70 (br s, 2H), 3.80-4.01 (m, 2H), 4.69 (s, 2H), 6.62 (d, J=8.7 Hz, 1H), 7.32-7.42 (m, 4H), 7.52-7.63 (m, 4H), 7.74 (d, J=8.1 Hz, 1H), 8.34 (s, 1H); ESI-MS (m/z) 466.24 (M+H)⁺.

### Example 34

### Ethyl-2-methylcarbonyloxy-5-(2-{6-[4-(2-trifluoromethylbenzoyl)piperazino-3-pyridyl}-1-ethynyl)benzoate

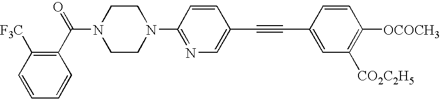

Prepared by Sonogashira coupling reaction of Intermediate 2 with ethyl 2-acetoxy-5-iodobenzoate to give the product as an off-white solid; IR (KBr) 2924, 2209, 1770, 1724, 1647, 1407, 1317, 1129 cm⁻¹; ¹H NMR (300 MHz, CDCl₃) δ 1.38 (t, J=6.9 Hz, 3H), 2.35 (s, 3H), 3.30 (br s, 2H), 3.75 (br s, 2H), 3.71 (br s, 2H), 3.85-4.01 (m, 2H), 4.37 (q, J=7.2 Hz, 2H), 6.63 (d, J=8.4 Hz, 1H), 7.07 (d, J=8.1 Hz, 1H), 7.37 (d, J=7.8 Hz, 1H), 7.56-7.63 (m, 4H), 7.74 (d, J=7.8 Hz, 1H), 8.14 (s, 1H), 8.35 (s, 1H); ESI-MS (m/z) 566.50 (M+H)⁺.

### Example 35

### 2-Hydroxy-5-(2-{6-[4-(2-trifluoromethylbenzoyl)piperazino]-3-pyridyl}-1-ethynyl)-benzoic acid

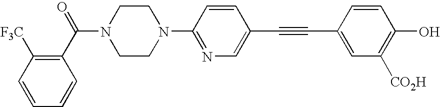

This compound was prepared by hydrolysis of Example 34 (100 mg, 0.1769 mmol) with 1N KOH (5 ml) in methanol (5 ml) followed by extractive work up with chloroform at pH 4 to give 60 mg of the product as an off-white solid (n-pentane); IR (KBr) 3433, 2213, 1642, 1499, 1244, 1126 cm⁻¹; ¹H NMR (300 MHz, CD₃OD) δ 3.49 (br s, 3H), 3.72 (br s, 3H), 3.85-3.95 (m, 2H), 6.81-6.87 (m, 2H), 7.46-7.50 (m, 2H), 7.62-7.82 (m, 4H), 7.95 (s, 1H), 8.24 (s, 1H); ESI-MS (m/z) 496.84 (M+H)⁺.

### Example 36

### N1-[3-(2-{6-[4-(2-Trifluoromethylbenzoyl)piperazino]-3-pyridyl}-1-ethynyl)phenyl]acetamide

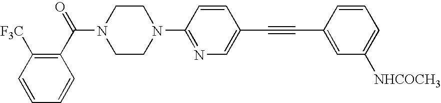

Prepared by Sonogashira coupling reaction of Intermediate 2 with N1-(3-iodophenyl)acetamide to give the product as an off-white solid; IR (KBr) 3268, 2204, 1698, 1595, 1498, 1124, 1011 cm⁻¹; ¹H NMR (300 MHz, CDCl₃) δ 2.05 (s, 3H), 3.17-380 (m, 8H), 6.88 (d, J=9.0 Hz, 1H), 7.17 (d, J=7.2 Hz, 1H), 7.32 (t, J=7.8 Hz, 1H), 7.47 (d, J=7.5 Hz, 1H), 7.55 (d, J=7.5 Hz, 1H), 7.65-7.86 (m, 5H), 8.32 (s, 1H), 10.04 (s, 1H); ESI-MS (m/z) 493.62 (M+H)⁺.

### Example 37

### {4-[6-[4-(2-Trifluoromethylbenzoyl)piperazin-1-yl]pyridazin-3-yl]ethynyl}phenol

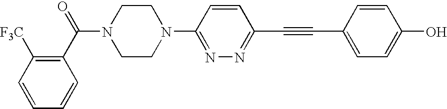

Step 1: 4-{(6-[4-(2-Trifluoromethylbenzoyl)piperazin-1-yl]pyridazin-3-yl)ethynyl}-phenyl acetate: Prepared by Sonogashira coupling reaction of Intermediate 6 with 4-iodophenyl acetate in a mixture of triethylamine and DMSO give the product as an off-white solid; ¹H NMR (300 MHz, CDCl₃) δ 2.31 (s, 3H), 3.35 (br s, 2H) 3.60-4.09 (m, 6H), 6.88 (d, J=8.1 Hz, 1H), 7.11 (d, J=8.1 Hz, 2H), 7.36-7.70 (m, 6H), 7.75 (d, J=7.2 Hz, 1H); ESI-MS (m/z) 495.40 (M+H)⁺.

Step 2: Deacetylation of Step 1 intermediate gave the product as an off-white solid (acetone); IR (KBr) 3435, 2205, 1644, 1431, 1246, 1117, 770 cm⁻¹; ¹H NMR (300 MHz, DMSO-d₆) δ 3.20-3.35 (m, 2H), 3.60-3.80 (m, 6H), 6.81 (d, J=8.7 Hz, 2H), 7.28 (d, J=9.6 Hz, 1H), 7.42 (d, J=8.1 Hz, 2H), 7.51-7.59 (m, 2H), 7.68-7.86 (m, 3H), 10.04 (s, 1H); ESI-MS (m/z) 453.95 (M+H)⁺.

### Example 38

### 4-{6-[2-(3-Hydroxyphenyl)-1-ethynyl]-3-pyridazinyl}piperazino-2-trifluoromethyl-phenylmethanone

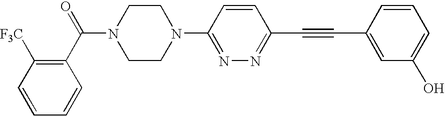

Step 1: 3-{(6-[4-(2-Trifluoromethylbenzoyl)piperazin-1-yl]pyridazin-3-yl)ethynyl}-phenyl acetate: Prepared by Sonogashira coupling reaction of Intermediate 6 with 3-iodophenyl acetate in a mixture of triethylamine and DMSO to give the product as an off-white solid; IR (KBr) 2926, 2216, 1640, 1434, 1316, 1116, 1010, 785 cm⁻¹; ¹H NMR (300 MHz, CDCl₃) δ 2.31 (s, 3H), 3.29-3.86 (m, 2H), 3.70-4.10 (m, 6H), 6.90 (d, J=9.9 Hz, 1H), 7.11 (d, J=7.2 Hz, 1H), 7.32-7.76 (m, 8H); ESI-MS (m/z) 454.88 (M)⁺.

Step 2: Deacetylation of Step 1 intermediate gave the product as an off-white solid; IR (KBr) 3400, 2216, 1640, 1590, 1434, 1316, 1244, 1116 cm⁻¹; ¹H NMR (300 MHz, DMSO-d₆) δ 3.15-3.26 (m, 2H), 3.60-3.90 (m, 6H), 6.85 (d, J=7.2 Hz, 1H), 6.94 (s, 1H), 7.01 (d, J=7.2 Hz, 1H), 7.22-7.31 (m, 2H), 7.55-7.70 (m, 3H), 7.76-7.86 (m, 2H), 9.73 (s, 1H); ESI-MS (m/z) 453.88 (M+H)⁺.

### Example 39

### 4-{5-[2-(4-Fluorophenyl)-1-ethynyl]-2-pyridyl}piperazino-2-trifluoromethylphenyl methanone

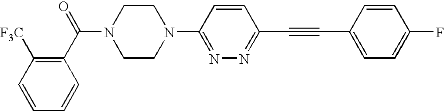

Prepared by Sonogashira coupling reaction of Intermediate 6 with 4-fluoroiodobenzene to give the product as an off-white solid; IR (KBr) 2949, 2073, 1645, 1427, 1382, 998, 755 cm⁻¹; ¹H NMR (300 MHz, CDCl₃) δ 3.75 (br s, 2H), 3.75 (br s, 4H), 3.89-4.05 (m, 2H), 6.88 (d. J=9.6 Hz, 1H), 7.06 (t, J=9.0 Hz, 2H), 7.39 (t, J=9.0 Hz, 2H), 7.54-7.60 (m, 4H), 7.75 (d, J=6.9 Hz, 1H); ESI-MS (m/z) 455.51 (M+H)⁺.

### Example 40

### 4-{6-[2-(3,4-Difluorophenyl)-1-ethynyl]-3-pyridazinyl}piperazino-2(trifluoromethyl)-phenylmethanone

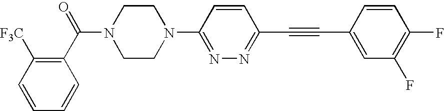

Prepared by Sonogashira coupling reaction of Intermediate 6 with 1,2-difluoro-4-iodobenzene in a mixture of triethylamine and DMSO to give the product as a white solid; IR (KBr) 2856, 2220, 1655, 1510, 1265, 1147, 1009 cm⁻¹; ¹H NMR (300 MHz, CDCl₃) δ 3.32-3.36 (m, 2H), 3.72-4.10 (m, 6H), 6.88 (d, J=9.3 Hz, 1H), 7.12-7.16 (m 1H), 7.28-7.65 (m, 6H), 7.73 (d, J=9.3 Hz, 1H) ESI-MS (m/z) 472.62 (M)⁺.

### Example 41

### 2-Trifluoromethylphenyl-4-{6-[2-(4-trifluoromethylphenyl)-1-ethynyl]-3-pyridazinyl}-piperazinomethanone

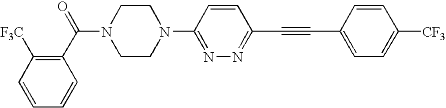

Prepared by Sonogashira coupling reaction of Intermediate 6 with 1-iodo-4-trifluoromethylbenzene in triethylamine to give the product as a white solid; IR (KBr) 2925, 1639, 1434, 1319, 1064, 846 cm⁻¹; ¹H NMR (300 MHz, CDCl₃) δ 3.56 (br s, 2H), 3.76-4.08 (m, 6H), 6.90 (d, J=9.6 Hz, 1H), 7.36-7.45 (m, 2H), 7.57-7.76 (m, 7H); ESI-MS (m/z) 505.58 (M+H)⁺.

### Example 42

### 4-{5-[2-(4-Hydroxyphenyl)-1-ethynyl]-2-pyrimidinyl}piperazino-2-trifluoromethyl-phenylmethanone

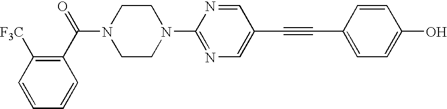

Step 1: 4-{(5-[4-(2-Trifluoromethylbenzoyl)piperazin-1-yl]pyrimidin-3-yl)ethynyl}-phenyl acetate: Prepared by Sonogashira coupling reaction of Intermediate 7 with 4-ethynylphenyl acetate in a mixture of triethylamine and DMSO to give the product as a brown solid; ¹H NMR (300 MHz, CDCl₃) δ 2.31 (s, 3H), 3.26 (br s, 2H), 3.80-4.00 (m, 6H), 7.19 (d, J=8.1 Hz, 2H), 7.37 (d, J=7.8 Hz, 1H), 7.51 (d, J=8.1 Hz, 2H), 7.56-7.64 (m, 2H), 7.75 (d, J=Hz, 1H), 8.44 (s, 2H).

Step 2: Deacetylation of Step 1 intermediate under basic conditions followed by recrystallisation of the crude material from acetone gave the product as an off-white solid; IR (KBr) 3238, 2214, 1621, 1608, 1518, 1317, 1255, 1008 cm⁻¹; ¹H NMR (300 MHz, CDCl₃) δ 3.15-3.29 (m, 2H), 3.72-3.88 (m, 6H), 6.78 (d, J=8.7 Hz, 2H), 7.34 (d, J=8.1 Hz, 2H), 7.55 (d, J=7.2 Hz, 1H), 7.67-7.86 (m, 3H), 8.54 (s, 2H), 9.95 (s, 1H); ESI-MS (m/z) 453.43 (M+H)⁺.

### Example 43

### 4-{5-[2-(3-Hydroxyphenyl)-1-ethynyl]-3-pyrimidinyl}piperazino-2-trifluoromethyl-phenylmethanone

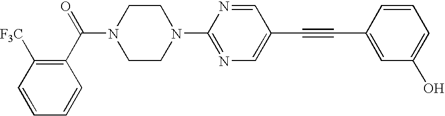

Step 1: 3-(2-{4-[4-(2-Trifluoromethylbenzoyl)piperazino]pyrimidinyl-1-ethynyl)phenyl acetate: Prepared by Sonogashira coupling reaction of Intermediate 7 with 3-ethynylphenyl acetate in a mixture of triethylamine and DMSO to give the product as a brown solid; IR (KBr) 2855, 2212, 1771, 1642, 1591, 1515, 1317, 1253 cm⁻¹; ¹H NMR (300 MHz, CDCl₃) δ 2.31 (s, 3H), 3.27 (br s, 2H), 3.80-4.00 (m, 6H), 7.05-7.07 (m, 1H), 7.25 (s, 1H), 7.36-7.38 (m, 3H), 7.56-7.61 (m, 2H), 7.75 (d, J=7.2 Hz, 1H), 8.44 (s, 2H).

Step 2: Deacetylation of Step 1 intermediate under basic conditions gave the product as an off-white solid; IR (KBr) 3287, 2205, 1626, 1588, 1440, 1316, 1255, 1115 cm⁻¹; ¹H NMR (300 MHz, CDCl₃) δ 3.26 (br s, 2H), 3.80-4.00 (m, 6H), 5.11 (br s, 1H), 6.82 (d, J=6.6 Hz, 1H), 6.97 (s, 1H), 7.21-7.26 (m, 1H), 7.08 (d, J=7.8 Hz, 1H), 7.37 (d, J=7.5 Hz, 1H), 7.56-7.76 (m, 3H), 8.44 (s, 2H); ESI-MS (m/z) 452.43 (M)⁺.

### Example 44

### Ethyl 5-(2-{6-[4-(2-trifluoromethylbenzoyl)piperazino]-2-pyridyl-1-ethynyl)nicotinate

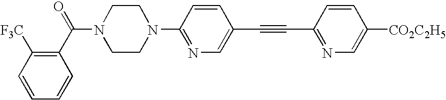

Prepared by Sonogashira coupling reaction of Intermediate 1 with ethyl 6-(1-ethynyl)nicotinate in a mixture of triethylamine and DMSO to give the product as a white solid; IR (KBr) 2864, 2208, 1719, 1629, 1585, 1503, 1437, 1124 cm⁻¹; ¹H NMR (300 MHz, CDCl₃) δ 1.42 (t, J=6.9 Hz, 3H), 3.28-3.22 (m, 2H), 3.59-3.62 (m, 2H), 3.70-3.77 (m, 2H), 3.88-3.99 (m, 2H), 4.42 (q, J=6.9 Hz, 2H), 6.63 (d, J=8.7 Hz, 1H), 7.37 (d, J=7.5 Hz, 1H), 7.54-7.76 (m, 5H), 8.27 (dd, J=1.8, 6.0 Hz, 1H), 8.43 (s, 1H), 9.18 (s, 1H); ESI-MS (m/z) 509.67 (M+H)⁺.

### Example 45

### 4-{5-[(2-Pyrazinyl-1-ethynyl)-2-pyridyl]}piperazino-2-trifluoromethylphenylmethanone

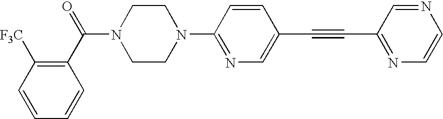

Prepared by Sonogashira coupling reaction of Intermediate 2 with 2-iodopyrazine to give the product as a white solid; IR (KBr) 2860, 2211, 1630, 1598, 1316, 1247, 1012, 777 cm⁻¹; ¹H NMR (300 MHz, CDCl₃) δ 3.32 (br s, 2H), 3.60 (br s, 2H), 3.74 (br s, 2H), 3.82-4.01 (m, 2H), 6.64 (d, J=9.0 Hz, 1H), 7.36 (d, J=7.5 Hz, 1H), 7.53-7.75 (m, 4H), 8.43 (d, J=11.4 Hz, 2H), 8.55 (s, 1H), 8.73 (s, 1H); ESI-MS (m/z) 438.47 (M+H)⁺.

### Example 46

### 2,5-Dichlorophenyl-4-{5-(2-pyrimidinyl)-1-ethynyl]-2-pyridyl}piperazinomethanone

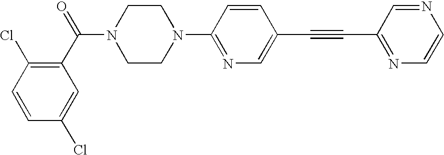

Prepared by Sonogashira coupling reaction of Intermediate 4 with 2-iodopyrazine to give the product as an off-white solid; ¹H NMR (300 MHz, CDCl₃) δ 3.34-3.43 (m, 2H), 3.66-4.01 (m, 6H), 6.65 (d, J=9.0 Hz, 1H), 7.32-7.39 (m, 3H), 7.70 (dd, J=2.4, 6.6 Hz, 1H), 8.45 (dd, J=7.2, 2.1 Hz, 1H), 8.47 (d, J=2.1 Hz, 1H), 8.56 (s, 1H), 8.73 (s, 1H); ESI-MS (m/z) 438.56 [100%, (M+H)⁺].

### Example 47

### 4-{5-[2-(1-Butyl-1H-2-imidazolyl)-1-ethynyl]-2-pyridyl}piperazino-2-trifluoromethyl-phenylmethanone

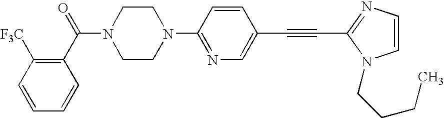

Prepared by Sonogashira coupling reaction of Intermediate 2 with 1-butyl-2-iodo-1H-imidazole in a mixture of triethylamine and DMSO to give the product as an off-white solid; IR (KBr) 2211, 1644, 1599, 1435, 1243, 1008 cm⁻¹; ¹H NMR (300 MHz, CDCl₃) δ 0.96 (t, J=7.2 Hz, 3H), 1.32-1.40 (m, 2H), 1.78-1.83 (m, 2H), 3.29 (br s, 2H), 3.58 (br s, 2H), 3.72 (br s, 2H), 3.89-3.97 (m, 2H), 4.07 (t, J=7.2 Hz, 2H), 6.62 (d, J=9.0 Hz, 1H), 6.94 (s, 1H), 7.08 (s, 1H), 7.37 (d, J=7.8 Hz, 1H), 7.55-7.63 (m, 3H), 7.74 (d, J=7.8 Hz, 1H), 8.36 (s, 1H); ESI-MS (m/z) 482.67 (M+H)⁺.

### Example 48

### 4-{5-[2-(1-(3-Methylbutyl)-1H-2-imidazolyl)-1-ethynyl]-2-pyridyl}piperazino-2-trifluoro-methylphenylmethanone

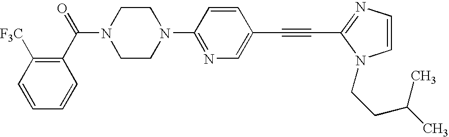

Prepared by Sonogashira coupling reaction of Intermediate 2 with 2-iodo-1-(3-methylbutyl)-1H-imidazole in triethylamine to give the product as an off-white solid; (KBr) 2960, 2215, 1600, 1243, 1132, 1008, 754 cm⁻¹; ¹H NMR (300 MHz, CDCl₃) δ 0.97 (t, J=6.3 Hz, 6H), 1.59-1.64 (m, 1H), 1.68-1.75 (m, 2H), 3.28-3.31 (m, 2H), 3.57-3.60 (m, 2H), 3.69-3.75 (m, 2H), 3.87-3.96 (m, 2H), 4.10 (t, J=6.0 Hz, 2H), 6.62 (d, J=9.3 Hz, 1H), 6.96 (s, 1H), 7.14 (s, 1H), 7.36 (d, J=7.2 Hz, 1H), 7.56-7.63 (m, 3H), 7.74 (d, J=8.1 Hz, 1H), 8.36 (s, 1H); ESI-MS (m/z) 496.82 (M+H)⁺.

### Example 49

### 4-{5-[2-(1H-5-Indolyl)-1-ethynyl]-2-pyridyl}piperazino-2-trifluoromethylphenyl-methanone

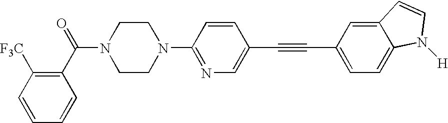

Prepared by Sonogashira coupling reaction of Intermediate 2 with 5-iodoindole to give the product as a white solid; IR (KBr) 3276, 2207, 1614, 1497, 1314, 1113 cm⁻¹; ¹H NMR (300 MHz, CDCl₃) δ 3.28-3.32 (m, 2H), 3.54-3.60 (m, 2H), 3.68 (br s, 2H), 3.90-4.00 (m, 2H), 6.55 (s, 1H), 6.63 (d, J=9.0 Hz, 1H), 7.23-7.26 (m, 1H), 7.35-7.38 (m, 3H), 7.52-7.73 (m, 3H), 7.74 (d, J=7.5 Hz, 1H), 7.84 (s, 1H), 8.26 (s, 1H), 8.36 (s, 1H); ESI-MS (m/z) 475.48 (M+H)⁺.

### Example 50

### 4-{5-[2-(1H-5-Indolyl)-1-ethynyl]-2-pyrimidinyl}piperazino-2-trifluoromethylphenyl-methanone

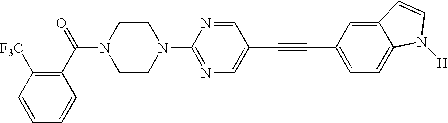

Prepared by Sonogashira coupling reaction of Intermediate 8 with 5-iodoindole in a mixture of triethylamine and DMSO to give the product as a light brown solid; IR (KBr) 3293, 2206, 1631, 1591, 1504, 1435, 1252, 1128, 1006 cm⁻¹; ¹H NMR (300 MHz, CDCl₃) δ 3.26 (t, J=5.4 Hz, 2H), 3.80 (t, J=5.4 Hz, 2H), 3.86-3.99 (m, 4H), 6.56 (s, 1H), 7.24 (s, 1H), 7.36-7.39 (m, 3H), 7.56-7.63 (m, 2H), 7.64 (d, J=7.5 Hz, 1H), 7.84 (s, 1H), 8.28 (br s, 1H), 8.47 (s, 2H); ESI-MS (m/z) 476.50 (M+H)⁺.

### Example 51

### 4-{5-[2-(4-(1,1-Dioxidoisothiazolidine-2-yl)phenyl)-1-ehtynyl]-2-pyrimidinyl}piperazino-2-trifluoromethylphenylmethanone

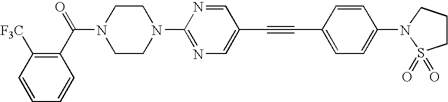

Prepared by Sonogashira coupling reaction of Intermediate 8 with 4-(1,1-dioxidoisothiazolidinynl)iodobenzene in a mixture of triethylamine and DMSO to give the product as a light brown solid; IR (KBr) 2859, 2210, 1625, 1593, 1515, 1435, 1254, 1140 cm⁻¹; ¹H NMR (300 MHz, CDCl₃) δ 2.54-2.60 (m, 2H), 3.26 (t, J=7.5 Hz, 2H), 3.41 (t, J=7.5 Hz, 2H), 3.76-4.01 (m, 8H), 7.21 (d, J=8.4 Hz, 2H), 7.37 (d, J=7.2 Hz, 2H), 7.49 (d, J=9.0 Hz, 1H), 7.50-7.63 (m, 2H), 7.34 (d, J=7.2 Hz, 1H), 8.44 (s, 2H); ESI-MS (m/z) 556.77 (M+H)⁺.

### Example 52

### 4-{5-[2-(4-(1H-1-Azolyl)phenyl)-1-ehtynyl]-2-pyrimidinyl}piperazino-2-trifluoro-methylphenylmethanone

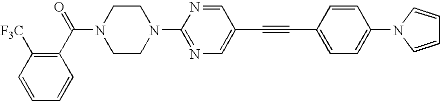

Prepared by Sonogashira coupling reaction of Intermediate 8 with 4-(1H-1-azolyl)phenyl iodide to give the product as a white solid; IR (KBr) 2924, 2209, 1632, 1591, 1520, 1435, 1317, 1254, 1118, 1010 cm⁻¹; ¹H NMR (300 MHz, CDCl₃) δ 3.27 (br s, 2H), 3.81-4.00 (m, 6H), 6.37 (s, 2H), 7.11 (s, 2H), 7.26-7.39 (m, 3H), 7.57-7.73 (m, 4H), 7.74-7.76 (m, 1H), 8.46 (s, 2H); ESI-MS (m/z) 502.32 (M+H)⁺.

### Example 53

### 4-(2-{2-[4-(2-Trifluoromethylbenzoyl)piperazino]-1,3-thiazol-5-yl}-1-ethynyl)phenyl acetate

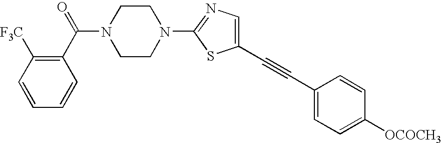

To a stirred suspension of Intermediate 9 (150 mg, 0.4109 mmol) and 4-iodophenyl acetate (161.5 mg, 0.6164 mmol) in THF (5 ml) was added PdCl₂(PPh₃)₂ (28.84 mg, 0.041 mmol) followed by Ag₂O (142.8 mg, 0.616 mmol). The mixture was stirred at 80° C. for 12 h under nitrogen atmosphere. Water (20 ml) was added and the mixture was extracted with ethyl acetate (2×20 ml). The combined organic layer was filtered through celite and washed with water (4×40 ml) and dried over Na₂SO₄. The crude product obtained after evaporation of the solvent was purified by silica gel column chromatography using 10% EtOAc in chloroform to give 41 mg of the product as a off white solid; IR (KBr) 2913, 2200, 1754, 1648, 1509, 1430, 1265, 1128, 1032 cm⁻¹; ¹H NMR (300 MHz, CDCl₃) δ 2.29 (s, 3H), 3.31 (br s, 2H), 3.45 (br s, 2H), 3.61 (br s, 2H), 3.90-4.00 (m, 2H), 7.05 (d, J=8.7 Hz, 2H), 7.34 (d, J=7.8 Hz, 1H), 7.35 (s, 1H), 7.45 (d, J=8.7 Hz, 2H), 7.54-7.61 (m, 2H), 7.31 (d, J=7.8 Hz, 1H); ESI-MS (m/z) 500.68 (M+H)⁺.

### Example 54

### 3-({6-[4-(Cyclopentylcarbonyl)piperazin-1-yl]pyridazin-3-yl}ethynyl)benzonitrile

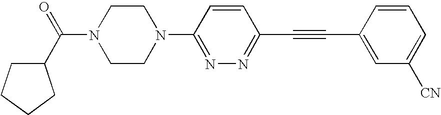

Prepared by Sonogashira coupling reaction of Intermediate 10 with 3-iodobenzonitrile in mixture of triethylamine and DMSO to give the product as an off-white solid; IR (KBr) 2948, 2864, 2231, 2211, 1633, 1582, 1230, 1019, 813 cm⁻¹; ¹H NMR (300 MHz, CDCl₃) δ 1.75-1.86 (m, 7H), 2.93 (m, 1H), 3.62 (m, 1H), 3.70 (m, 2H), 3.80 (m, 3H), 3.89 (m, 2H), 6.86 (m, J=9.0 Hz, 1H), 7.39 (d, J=9.0 Hz, 2H), 7.44 (m, 1H), 7.62 (d, J=6.0 Hz, 1H), 7.76 (m, 2H); ESI-MS (m/z) 386.54 (M+H)⁺.

### Example 55

### 3-({6-[4-(Cyclopropylmethyl)piperazin-1-yl]pyridazin-3-yl}ethynyl)phenol

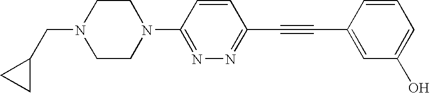

Step 1: 3-({6-[4-(Cyclopropylmethyl)piperazin-1-yl]pyridazin-3-yl}ethynyl)phenyl acetate: Prepared by a Sonogashira coupling reaction of Intermediate 11 with 3-ethynylphenyl acetate in mixture of triethylamine and DMSO to give the product as a white solid; IR (KBr) 2946, 2867, 2781, 1758, 1441, 1238 cm⁻¹; ¹H NMR (300 MHz, CDCl₃) δ 0.24 (m, 2H), 0.63-0.65 (m, 2H), 1.04 (m, 1H), 2.31 (s, 3H), 2.51 (s, 2H), 2.87 (s, 4H), 3.92 (s, 4H), 6.84 (d, J=9.0 Hz, 1H), 7.08 (d, J=9.0 Hz, 1H), 7.30-7.44 (m, 4H); ESI-MS (m/z) 377.26 (M+H)⁺.

Step 2: Deacetylation of Step 1 intermediate gave the product as an off-white solid; IR (KBr) 3377, 2960, 2887, 2844, 1576, 1434, 1243, 786 cm⁻¹; ¹H NMR (300 MHz, CDCl₃) δ 0.09 (d, J=4.5 Hz, 2H), 0.48 (d, J=6.9 Hz, 2H), 0.86 (br s, 1H), 2.22 (d, J=6.3 Hz, 2H), 2.51 (d, J=15.0 Hz, 4H), 3.41 (s, 1H), 3.65 (s, 4H), 6.82 (d, J=9.0 Hz, 1H), 6.91 (m, 2H), 7.22 (m, 2H), 7.53 (d, J=9.0 Hz, 1H); ESI-MS (m/z) 335.27 (M+H)⁺.

### Example 56

### 3-([6-{(4-Cyclohexylmethyl)piperazin-1-yl]pyridazin-3-yl}ethynyl)phenyl acetate

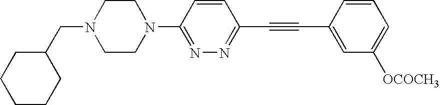

Prepared by Sonogashira coupling reaction of Intermediate 12 with 3-(1-ethynyl)phenyl acetate in mixture of triethylamine and DMSO to give the product as an off-white solid; IR (KBr) 2952, 2220, 1582, 1439, 1260, 1232, 1016, 739 cm⁻¹; ¹H NMR (300 MHz, CDCl₃) δ 0.86-0.90 (m, 2H), 1.22-1.26 (m, 3H), 1.59 (br s, 1H), 1.67-1.81 (m, 5H), 2.15 (d, J=6.3 Hz, 2H), 2.30 (s, 3H), 2.50 (t, J=4.8 Hz, 4H), 3.74-3.88 (m, 4H), 6.81 (d, J=9.0 Hz, 1H), 7.05 (d, J=9.0 Hz, 1H), 7.24-7.36 (m, 2H), 7.43 (d, J=9.0 Hz, 2H); ESI-MS (m/z) 419.67 (M+H)⁺.

### Example 57

### 3-({6-[4-(Cyclohexylmethyl)piperazin-1-yl]pyridazin-3-yl}ethynyl)phenol

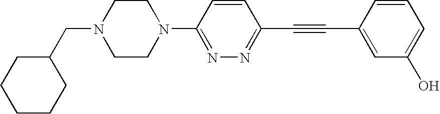

Deacetylation of Example 56 (150 mg, 0.358 mmol) afforded 98 mg of the product as an off-white solid; IR (KBr) 3436, 2923, 2848, 2213, 1590, 1439, 1256 cm⁻¹; ¹H NMR (300 MHz, CDCl₃) δ 0.84-0.94 (m, 2H), 1.17-1.27 (m, 3H), 1.60-1.70 (m, 1H), 1.73-1.85 (m, 5H), 2.11 (d, J=7.2 Hz, 2H), 2.42 (s, 5H), 3.63 (s, 4H), 6.82 (d, J=9.0 Hz, 1H), 6.91-7.02 (m, 2H), 7.21-7.38 (m, 2H), 7.53 (d, J=9.0 Hz, 1H); ESI-MS (m/z) 377.74 (M+H)⁺.

### Example 58

### 3-{4-[(2-Fluorobenzyl)piperazin-1-yl]-6-(tetrahydro-2H-pyran-2-ylethynyl)}pyridazine

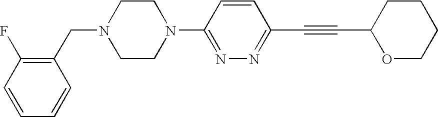

Prepared by Sonogashira coupling reaction of intermediate 13 with 2-ethynyltetrahydro-2H-pyran in mixture of triethylamine and DMSO to give the product as a white solid; IR (KBr) 2947, 1586, 1431, 1259, 1082, 759 cm⁻¹; ¹H NMR (300 MHz, CDCl₃) δ 1.50-1.70 (m, 4H), 1.80-1.90 (m, 1H), 1.90-2.00 (m, 2H), 2.60 (br s, 4H), 3.63 (s, 2H), 3.62-3.70 (m, 4H), 4.02 (br s, 1H), 4.50-4.62 (m, 1H), 6.75 (d, J=9.3 Hz, 1H), 6.98-7.18 (m, 2H), 7.20-7.30 (m, 2H), 7.34-7.50 (m, 1H); ESI-MS (m/z) 381.64 (M+H)⁺.

### Example 59

### 4-[{6-[4-(2-Fluorobenzyl)piperazin-1-yl]pyridazin-3-yl}ethynyl]phenyl acetate

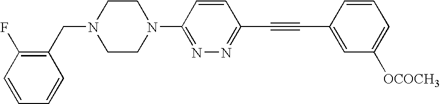

Prepared by Sonogashira coupling reaction of Intermediate 14 with 3-iodophenyl acetate in a mixture of triethylamine and DMSO to give the product as a white solid; IR (KBr) 2937, 1760, 1582, 1200, 998, 753 cm⁻¹; ¹H NMR (300 MHz, CDCl₃) δ 2.30 (s, 3H), 2.60 (br s, 4H), 3.48 (s, 2H), 3.64 (s, 2H), 3.65-3.76 (m, 3H), 3.79 (d, J=9.9 Hz, 1H), 6.98-7.18 (m, 3H), 7.20-7.45 (m, 5H); ESI-MS (m/z) 431.37 (M+H)⁺.

### Example 60

### 3-({6-[4-(2-Fluorobenzyl)piperazin-1-yl]pyridazin-3-yl}ethynyl)phenol

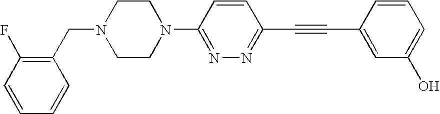

Deacetylation of Example 59 gave the product as an off-white solid; IR (KBr) 3421, 2944, 2848, 2215, 1587, 1438, 1257, 765 cm⁻¹; ¹H NMR (300 MHz, CDCl₃) δ 3.27 (s, 2H), 3.62 (d, J=18.0 Hz, 8H), 6.82 (d, J=6.0 Hz, 1H), 6.91 (s, 1H), 6.98 (d, J=6.0 Hz, 1H), 7.13-7.31 (m, 4H), 7.31-7.40 (m, 1H), 7.43-7.50 (m, 1H), 7.53 (d, J=9.0 Hz, 1H), 9.75 (s, 1H); ESI-MS (m/z) 389.68 (M+H)⁺.

### Example 61

### 4-{[6-(4-Benzyl-4-hydroxypiperidin-1-yl)pyridazin-3-yl]ethynyl}phenyl acetate

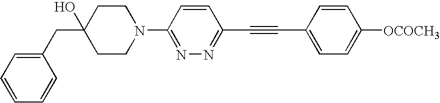

Prepared by Sonogashira coupling reaction of Intermediate 15 with 4-ethnylphenyl acetate to give the product as a white solid; IR (KBr) 3478, 2945, 1760, 1431, 1202, 1012, 836 cm⁻¹; ¹H NMR (300 MHz, CDCl₃) δ 1.59 (s, 1H, D₂O exchangeable), 1.62-1.80 (m, 4H), 2.30 (s, 3H), 2.78 (s, 2H), 3.37 (t, J=13.2 Hz, 2H), 4.22 (d, J=11.7 Hz, 2H), 6.83 (d, J=8.7 Hz, 1H), 7.07 (d, J=7.2 Hz, 2H), 7.18 (d, J=6.3 Hz, 2H), 7.22-7.38-7.44 (m, 4H), 7.56 (d, J=7.2 Hz, 2H); ESI-MS (m/z) 428.29 (M+H)⁺.

### Example 62

### 4-Benzyl-1-{6-[(4-hydroxyphenyl)ethynyl]pyridazin-3-yl}piperidin-4-ol

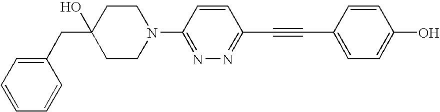

Deacetylation of Example 61 gave the product as a white solid; IR (KBr) 3566, 2932, 2210, 1606, 1539, 1258, 1084, 845 cm⁻¹; ¹H NMR (300 MHz, DMSO-d₆) δ 1.47 (br s, 4H), 2.70 (s, 2H), 3.27 (br s, 2H), 4.10 (d, J=13.8 Hz, 2H), 4.50 (s, 1H, D₂O exchangeable), 4.78 (d, J=7.8 Hz, 2H), 7.10-7.30 (m, 6H), 7.38-7.50 (m, 3H), 9.97 (s, 1H).

### Example 63

### 4-(2-Fluorobenzyl)-1-{6-[(4-hydroxyphenyl)ethynyl]pyridazin-3-yl}piperidin-4-ol

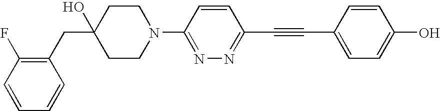

Step 1: 4-{[6-(4-(2-Fluorobenzyl-4-hydroxypiperidin-1-yl)pyridazin-3-yl]ethynyl}phenyl acetate: Prepared by Sonogashira coupling reaction of Intermediate 16 with 4-ethynylphenyl acetate to give the product as a white solid; IR (KBr) 2949, 2212, 1767, 1436, 1191, 1012, 838 cm⁻¹; ¹H NMR (300 MHz, CDCl₃) δ 1.45 (s, 1H, D₂O exchangeable), 1.55-1.85 (m, 4H), 2.30 (s, 3H), 2.85 (s, 2H), 3.30-3.50 (m, 2H), 4.18-4.30 (m, 2H), 6.83 (d, J=8.7 Hz, 1H), 7.02-7.18 (m, 6H), 7.30 (d, J=9.3 Hz, 1H), 7.56 (d, J=8.4 Hz, 1H).

Step 2: Deacetylation of Step 1 intermediate gave the product as a white solid; IR (KBr) 3548, 2936, 2211, 1606, 1454, 1232, 1094, 758 cm⁻¹; ¹H NMR (300 MHz, DMSO-d₆) δ 1.50 (br s, 4H), 2.74 (s, 2H), 3.33 (br s, 2H), 4.11 (br s, 2H), 4.60 (s, 1H, D₂O exchangeable), 6.78 (d, J=8.7 Hz, 2H), 7.00-7.20 (m, 2H), 7.24-7.50 (m, 6H), 9.97 (s, 1H).

### Example 64

### 4-{[6-(4-Hydroxy-4-[(2,5-dichlorobenzyl)piperidin-1-yl)pyridazin-3-yl]ethynyl}phenyl acetate

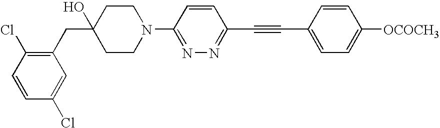

Prepared by Sonogashira coupling reaction of Intermediate 17 with 4-ethynylphenyl acetate to give the product as a white solid; IR (KBr) 2918, 2215, 1766, 1442, 1192, 919 cm⁻¹; ¹H NMR (300 MHz, CDCl₃) δ 1.58-1.74 (m, 2H), 1.69 (br s, 1H), 1.78-1.90 (m, 2H), 2.30 (s, 3H), 2.96 (s, 2H), 3.36 (t, J=12.3 Hz, 2H), 4.26 (d, J=13.2 Hz, 2H), 6.84 (d, J=9.6 Hz, 1H), 7.07 (d, J=8.7 Hz, 2H), 7.16 (dd, J=5.7, 2.4 Hz, 1H), 7.25-7.34 (m, 3H), 7.56 (d, J=8.7 Hz, 2H); ESI-MS (m/z) 497.35 [100%, (M+H)⁺].

### Example 65

### 1-{6-[(4-Hydroxyphenyl)ethynyl]pyridazin-3-yl}-4-(2,5-dichlorobenzyl)piperidin-4-ol

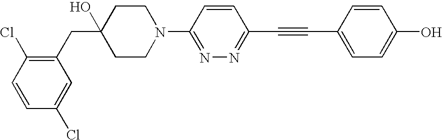

Deacetylation of Example 64 gave the product as a white solid; IR (KBr) 3528, 2938, 2211, 1606, 1437, 1255, 916 cm⁻¹; ¹H NMR (300 MHz, CDCl₃) δ 1.40-1.64 (m, 4H), 2.87 (s, 2H), 3.18-3.30 (m, 2H), 4.17 (d, J=13.2 Hz, 2H), 4.72 (s, 1H), 6.79 (d, J=8.4 Hz, 2H), 7.18-7.56 (m, 7H), 9.97 (s, 1H); ESI-MS (m/z) 454.38 [100%, (M)⁺].

### Example 66

### 4-[{6-[3-(2-Fluorophenoxyazetidin-1-yl)pyridazin-3-yl}ethynyl]phenyl acetate

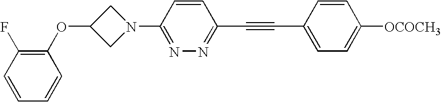

Prepared by Sonogashira coupling reaction of Intermediate 18 with 4-ethynylphenyl acetate to give the product as an off-white solid; IR (KBr) 2936, 2217, 1750, 1445, 1202, 745 cm⁻¹; ¹H NMR (300 MHz, DMSO-d₆) δ 2.30 (s, 3H), 4.32 (br s, 2H), 4.58 (br s, 2H), 5.18 (br s, 1H), 6.53 (d, J=9.0 Hz, 2H), 6.70-6.84 (m, 1H), 6.90-7.00 (m, 1H), 7.04-7.20 (m, 4H), 7.35 (d, J=9.3 Hz, 1H), 7.57 (d, J=8.4 Hz, 1H); ESI-MS (m/z) 404.48 (M+H)⁺.

### Example 67

### 4-[{6-[3-(2-Fluorophenoxyazetidin-1-yl)pyridazin-3-yl}ethynyl]phenol

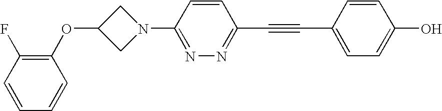

Deacetylation of Example 66 gave the product as an off-white solid; IR (KBr) 3434, 2938, 2212, 1606, 1471, 1257, 1034, 757 cm⁻¹; ¹H NMR (300 MHz, DMSO-d₆) δ 3.39 (br s, 1H, D₂O exchangeable), 4.10 (d, J=8.7 Hz, 2H), 4.50-4.64 (m, 2H), 5.26 (br s, 1H), 6.70-6.90 (m, 4H), 6.96-7.10 (m, 2H), 7.12-7.20 (m, 1H), 7.22-7.36 (m, 1H), 7.36-7.50 (m, 1H), 7.52 (d, J=9.3 Hz, 1H); ESI-MS (m/z) 362.41 (M+H)⁺.

### Example 68

### 3-(2-{6-[(3S)-3-(2-Fluorophenoxy)azolan-1-yl]-3-pyridazinyl}-1-ethynyl)phenyl acetate

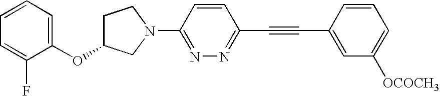

Prepared by Sonagashira coupling reaction of Intermediate 20 with 3-iodophenyl acetate in a mixture of DMSO and triethylamine to give the product as a white solid; IR (KBr) 2938, 2208, 1762, 1578, 1504, 1371, 1240, 1109, 758 cm⁻¹; ¹H NMR (300 MHz, CDCl₃) δ 2.26 (br s, 1H), 2.30 (s, 3H), 2.48 (br s, 1H), 3.76-4.02 (m, 4H), 5.11 (br s, 1H), 6.61 (d, J=9.3 Hz, 1H), 6.94-7.10 (m, 4H), 7.29-7.36 (m, 4H), 7.42 (d, J=9.3 Hz, 1H); ESI-MS (m/z) 418.12 (M+H)⁺.

### Example 69

### 3-(2-{6-[(3S)-3-(2-Fluorophenoxy)azolan-1-yl]-3-pyridyl}-1-ethynyl)phenol

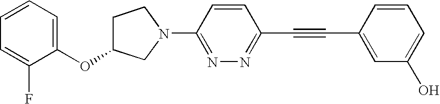

Deacetylation of Example 68 gave the product as an off-white solid; IR (KBr) 3340, 2866, 2209, 1591, 1546, 1455, 1203, 1111, 748 cm⁻¹; ¹H NMR (300 MHz, DMSO-d₆) δ 2.30 (br s, 2H), 3.57-3.81 (m, 4H), 5.25 (br s, 1H), 6.82 (d, J=9.3 Hz, 1H), 6.92-6.99 (m, 4H), 7.12-7.29 (m, 4H), 7.54 (d, J=9.3 Hz, 1H), 9.81 (br s, 1H); ESI-MS (m/z) 376.38 (M+H)⁺.

### Example 70

### 4-[{6-[(3S)-3-(2-Fluorophenoxy)azolan-1-yl]pyridazin-3-yl}ethynyl]phenyl acetate

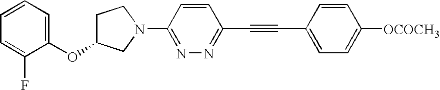

Prepared by Sonogashira coupling reaction of Intermediate 19 with 4-ethynylphenyl acetate in triethylamine to give the product as an off-white solid; IR (KBr) 2952, 2220, 1582, 1439, 1260, 1016, 739 cm⁻¹; ¹H NMR (300 MHz, DMSO-d₆) δ 2.18-2.38 (m, 1H), 2.30 (s, 3H), 2.40-2.56 (m, 1H), 3.70-3.90 (m, 3H), 3.94-4.10 (m, 1H), 5.11 (br s, 1H), 6.60 (d, J=9.3 Hz, 1H), 6.90-7.18 (m, 6H), 7.33 (d, J=9.3 Hz, 2H), 7.56 (d, J=8.4 Hz, 1H); ESI-MS (m/z) 418.39 (M+H)⁺.

### Example 71

### 4-[{6-[(3S)-3-(2-Fluorophenoxy)azolan-1-yl]pyridazin-3-yl}ethynyl]phenol

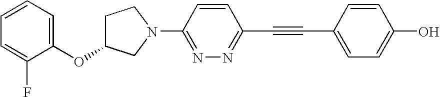

Deacetylation of Example 70 gave the product as an off-white solid; IR (KBr) 3431, 2950, 2211, 1607, 1456, 1275, 1033, 741 cm⁻¹; ¹H NMR (300 MHz, DMSO-d₆) δ 2.27 (br s, 2H), 3.37 (br s, 1H, D₂O exchangeable), 3.42-3.60 (m, 2H), 3.64-3.88 (m, 2H), 5.23 (br s, 1H), 6.77 (d, J=8.4 Hz, 2H), 6.88-7.02 (m, 2H), 7.10-7.30 (m, 3H), 7.36 (d, J=8.7 Hz, 2H), 7.48 (d, J=9.3 Hz, 1H); ESI-MS (m/z) 376.31 (M+H)⁺.

### Example 72

### 1-[5-(2-Benzo[d][1,3]dioxol-5-yl-1-ethynyl)-2-pyridyl-4-(2-fluorophenoxy)piperidine

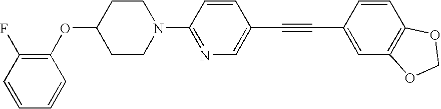

Prepared by a Sonogashira coupling reaction of Intermediate 21 with 5-(1-ethynyl)benzo[d][1,3]dioxole in triethylamine to give the product as an off-white solid; IR (KBr) 2822, 2203, 1592, 1495, 1395, 1236, 1033 cm⁻¹; ¹H NMR (300 MHz, CDCl₃) δ 1.90-2.02 (m, 4H), 3.46-3.54 (m, 2H), 3.93-3.98 (m, 2H), 4.51 (br s, 1H), 5.97 (s, 2H), 6.62 (d, J=8.7 Hz, 1H), 6.76 (d, J=7.8 Hz, 1H), 6.94-7.11 (m, 6H), 7.54 (dd, J=6.6, 2.1 Hz, 1H), 8.30 (s, 1H); ESI-MS (m/z) 417.61 (M+H)⁺.

### Example 73

### 4-(2-Fluorophenoxy)-1-{5-[2-(3-pyridyl)-1-ethynyl]-2-pyridyl}piperidine

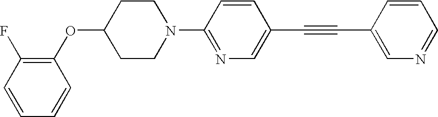

Prepared by Sonogashira coupling reaction of Intermediate 22 with 3-iodopyridine to give the product as an off-white solid; IR (KBr) 2849, 2208, 1603, 1497, 1046, 1258, 1029, cm⁻¹; ¹H NMR (300 MHz, DMSO-d₆) δ 1.65 (br s, 2H), 1.98 (br s, 2H), 3.74 (br s, 2H), 3.99 (br s, 2H), 4.66 (br s, 1H), 6.91-6.94 (m, 2H), 7.29 (m, 3H), 7.41 (t, J=5.4 Hz, 1H), 7.67 (d, J=9.0 Hz, 1H), 7.90 (d, J=7.8 Hz, 1H), 8.31 (s, 1H), 8.51 (s, 1H), 8.68 (s, 1H); ESI-MS (m/z) 374.38 (M+H)⁺.

### Example 74

### 4-(2-Fluorophenoxy)-1-(5-{2-[3-(1-oxo)pyridyl]-1-ethynyl}-2-pyridyl)piperidine

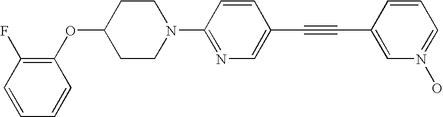

Prepared by Sonogashira coupling reaction of Intermediate 22 with 1-oxo-3-iodopyridine to give the product as an off-white solid; IR (KBr) 2954, 2211, 1597, 1499, 1258, 1009, 756 cm⁻¹; ¹H NMR (300 MHz, CDCl₃) δ 1.89-2.02 (m, 4H), 3.53-3.60 (m, 2H), 3.93-3.99 (m, 2H), 4.54 (br s, 1H), 6.63 (d, J=9.0 Hz, 1H), 6.92-7.23 (m, 5H), 7.33 (d, J=7.5 Hz, 1H), 7.54 (d, J=9.3 Hz, 1H), 8.10 (d, J=6.3 Hz, 1H), 8.27 (s, 1H), 8.32 (s, 1H); ESI-MS (m/z) 390.55 (M+H)⁺.

### Example 75

### 4-[{6-[4-(2-Fluorophenoxy)piperidin-1-yl]pyridazin-3-yl}ethynyl]phenyl acetate

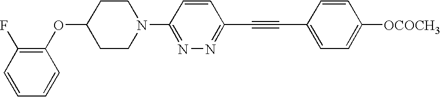

Prepared by Sonagashira coupling reaction of Intermediate 23 with 4-ethynylphenyl acetate to give the product as an off-white solid; IR (KBr) 2948, 2220, 1761, 1586, 1438, 1258, 1023, 742 cm⁻¹; ¹H NMR (300 MHz, CDCl₃) δ 1.84-2.16 (m, 4H), 2.31 (s, 3H), 3.64-3.78 (m, 2H), 3.90-4.10 (m, 2H), 4.58 (br s, 1H), 6.84-7.18 (m, 7H), 7.35 (d, J=9.6 Hz, 1H), 7.59 (d, J=8.4 Hz, 2H); ESI-MS (m/z) 432.52 (M+H)⁺.

### Example 76

### 4-(2-{6-[4-(2-Fluorophenoxy)piperidino]-3-pyridazinyl}-1-ethynyl)phenol

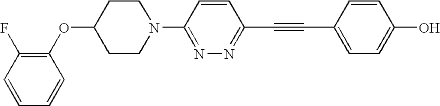

Deacetylation of Example 75 gave the product as an off-white solid; IR (KBr) 3413, 2929, 2209, 1606, 1542, 1447, 1272, 1023, 743 cm⁻¹; ¹H NMR (300 MHz, DMSO-d₆) δ 1.69 (br s, 2H), 2.01 (br s, 2H), 3.52 (br s, 2H), 4.04 (br s, 2H), 4.69 (br s, 1H), 6.82 (d, J=8.7 Hz, 2H), 6.97-7.01 (m, 1H), 7.11-7.33 (m, 4H), 7.42 (d, J=9.0 Hz, 2H), 7.53 (d, J=9.3 Hz, 1H), 10.03 (s, 1H); ESI-MS (m/z) 390.43 (M+H)⁺.

### Example 77

### 4-(2-{6-[4-(2-Fluorophenoxy)piperidin-1-yl]pyridazin-3-yl}ethynyl)phenol potassium

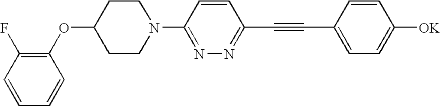

To a stirred solution of Example 76 (200 mg, 0.514 mmol) in a mixture of MeOH (10 ml) and tetrahydrofuran (10 ml) was added KOH (33 mg, 0.514 mmol) in MeOH (1.0 ml) at room temperature. The reaction mixture was stirred for 1 h at the same temperature. The solvent was evaporated under reduced pressure and the residue obtained was triturated with diethyl ether to give 150 mg of the product as a yellow solid; IR (KBr) 2929, 2201, 1594, 1454, 1282, 1042, 745 cm⁻¹; ¹H NMR (300 MHz, CDCl₃) δ 1.66 (br s, 2H), 2.00 (br s, 2H), 3.20-3.56 (m, 2H), 4.00 (brs, 2H), 4.66 (s, 1H), 6.00 (d, J=8.4 Hz, 2H), 6.92 (d, J=8.4 Hz, 3H), 7.08-7.38 (m, 5H); ESI-MS (m/z) 428.33 (M+H)⁺.

### Example 78

### 3-[4-(2-Fluorophenoxy)piperidin-1-yl]-1-{[4-piperidin-1-ylethoxy]phenylethynyl}-pyridazine

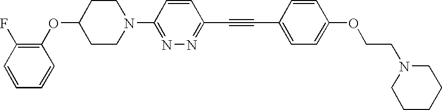

To a stirred solution of Example 76 (200 mg, 0.514 mmol) in dry DMF (5 ml) was added 1-(2-chloroethyl)piperidine monohydrochloride (142 mg, 0.771 mmol) and K₂CO₃ (178 mg, 1.285 mmol) under nitrogen at room temperature. The reaction mixture was stirred overnight at the same temperature. The mixture was diluted with water (50 ml) and extracted with EtOAc (2×50 ml). The combined organic extracts were washed with water (50 ml) and dried over anhydrous Na₂SO₄. The crude product obtained after evaporation of the solvent was purified by silica gel column chromatography using 5% MeOH in chloroform to give 165 mg of the product as an off-white solid; IR (KBr) 2923, 2338, 1601, 1492, 1028, 757 cm⁻¹; ¹H NMR (300 MHz, CDCl₃) δ 1.40-1.50 (m, 3H), 1.50-1.70 (m, 3H), 1.84-2.14 (m, 4H), 2.52 (br s, 4H), 2.79 (t, J=6.0 Hz, 2H), 3.62-3.74 (m, 2H), 3.96-4.10 (m, 2H), 4.12 (t, J=6.0 Hz, 2H), 4.55 (br s, 1H), 6.82-7.00 (m, 4H), 7.00-7.16 (m, 3H), 7.31 (d, J=9.6 Hz, 1H), 7.48 (d, J=9.0 Hz, 2H); ESI-MS (m/z) 501.31 (M+H)⁺.

### Example 79

### 4-{[6-[4-(2-Fluorophenoxy)piperidin-1-yl]pyridazin-3-yl]ethynylphenoxymorpholine

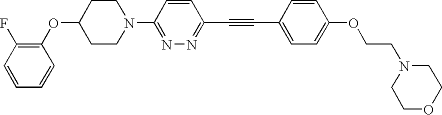

To a stirred solution of solution of Example 76 (200 mg, 0.514 mmol) in dry DMF (5 ml) was added 4-(2-chloroethyl)morpholine monohydrochloride (144 mg, 0.565 mmol) and K₂CO₃ (178 mg, 1.287 mmol) under nitrogen at room temperature. The reaction mixture was stirred overnight at the same temperature. The mixture was diluted with water (50 ml) and extracted with EtOAc (2×50 ml). The combined organic extracts were washed with water (50 ml) and dried over anhydrous Na₂SO₄. The crude product obtained after evaporation of the solvent was purified by silica gel column chromatography using 40% EtOAc in chloroform to give 80 mg of the product as an off-white solid; IR (KBr) 2927, 2211, 1604, 1248, 1115, 743 cm⁻¹; ¹H NMR (300 MHz, CDCl₃) δ 1.88-2.10 (m, 4H), 2.58 (t, J=4.5 Hz, 4H), 2.80 (t, J=5.7 Hz, 2H), 3.60-3.78 (m, 6H), 3.86-4.10 (m, 1H), 4.12 (t, J=6.0 Hz, 2H), 4.50-4.60 (m, 1H), 6.86 (d, J=8.4 Hz, 2H), 6.90-7.14 (m, 5H), 7.31 (d, J=9.3 Hz, 1H), 7.48 (d, J=9.0 Hz, 2H); ESI-MS (m/z) 503.40 (M+H)⁺.

### Example 80

### 4-{6-[4-(2-Fluorophenoxy)piperidin-1-yl]pyridazin-3-yl}ethynylphenyl-2-furoate

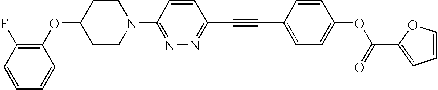

To a stirred solution of Example 76 (200 mg, 0.514 mmol) in dry THF (5 ml) was added triethylamine (77 mg, 0.771 mmol) and 2-furoyl chloride (74 mg, 0.565 mmol) under nitrogen at room temperature. The mixture was stirred for 2 h at the same temperature and diluted with water (50 ml). The mixture was extracted with EtOAc (2×50 ml) and the combined organic extracts were washed with water (50 ml) and dried over anhydrous Na₂SO₄. The crude product obtained after evaporation of the solvent was purified by silica gel column chromatography using 10% EtOAc in chloroform to give 180 mg of the product as a white solid; IR (KBr) 2936, 2218, 1747, 1503, 1174, 1081, 748 cm⁻¹; ¹H NMR (300 MHz, CDCl₃) δ 1.88-2.10 (m, 4H), 3.62-3.78 (m, 2H), 3.96-4.10 (m, 2H), 4.56 (br s, 1H), 6.54-6.64 (m, 1H), 6.92-7.12 (m, 6H), 7.18-7.30 (m, 2H), 7.34-7.44 (m, 1H), 7.58-7.70 (m, 3H); ESI-MS (m/z) 484.33 (M+H)⁺.

### Example 81

### 4-(2-{6-[4-Bromo-2-fluorophenoxy)piperidino]-3-pyridazinyl}-1-ethynyl)phenol

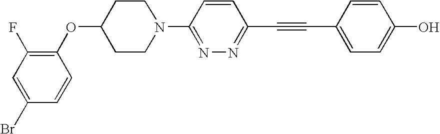

Step 1: 4-(2-{6-[4-Bromo-2-fluorophenoxy)piperidino]-3-pyridazinyl}-1-ethynyl)phenyl acetate: Prepared by Sonogashira coupling reaction of Intermediate 25 with 4-iodophenyl acetate in a mixture triethylamine and DMSO to give the product as an off-white solid; ¹H NMR (300 MHz, CDCl₃) δ 1.86-2.04 (m, 4H), 2.32 (s, 3H), 3.69-3.74 (m, 2H), 3.96-4.02 (m, 2H), 4.54 (br s, 1H), 6.86-6.92 (m, 2H), 7.08 (d, J=8.1 Hz, 2H), 7.16-7.26 (m, 2H), 7.35 (d, J=9.0 Hz, 1H), 7.57 (d, J=8.4 Hz, 2H); ESI-MS (m/z) 510.40 (M+H)⁺.

Step 2: Deacetylation of Step 1 gave the product as an off-white solid; IR (KBr) 3432, 2957, 2214, 1606, 1495, 1285, 1021, 845 cm⁻¹; ¹H NMR (300 MHz, DMSO-d₆) δ 1.55 (br s, 1H), 1.68 (br s, 2H), 2.02 (br s, 2H), 3.50 (br s, 2H), 4.03 (br s, 2H), 4.70 (br s, 1H), 6.79 (d, J=8.4 Hz, 2H), 7.26-7.34 (m, 3H), 7.39 (d, J=8.1 Hz, 2H) 7.48-7.56 (m, 2H).

### Example 82

### 2-Fluoro-[4-{6-(4-[2-fluorophenoxy]piperidin-1-yl)pyridazin-3-yl}ethynylphenol

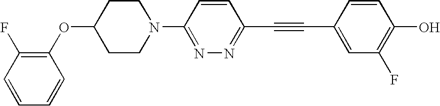

Step 1: 2-Fluoro-4-({6-[4-(2-fluorophenoxy)piperidin-1-yl]pyridazin-3-yl}ethynyl)phenyl acetate: Prepared by a Sonogashira coupling reaction of Intermediate 23 with 4-ethynyl-2-fluorophenyl acetate in a mixture of triethylamine and DMSO to give the product as a white solid; IR (KBr) 2932, 2213, 1765, 1584, 1257, 1176, 1048, 742 cm⁻¹; ¹H NMR (300 MHz, CDCl₃) δ 2.01 (br s, 3H), 2.34 (s, 4H), 3.75 (br s, 2H), 3.99 (br s, 2H), 4.58 (br s, 1H), 6.91-6.94 (m, 2H), 7.03-7.13 (m, 4H), 7.33-7.35 (m, 3H).

Step 2: Deacetylation of Step 1 gave the product as an off-white solid; IR (KBr) 3421, 2960, 2208, 1589, 1542, 1260, 1197, 1024, 756 cm⁻¹; ¹H NMR (300 MHz, CDCl₃) δ 1.69 (br s, 2H), 2.01 (br s, 2H), 3.51 (s, 1H, D₂O exchangeable), 3.70 (br s, 2H), 4.03 (br s, 2H), 4.68 (br s, 1H), 6.80-7.05 (m, 2H), 7.08-7.38 (m, 6H), 7.49 (d, J=9.61 Hz, 1H); ESI-MS (m/z) 408.35 (M+H)⁺.

### Example 83

### 2-Methoxy-4-{6-[4-(2-fluorophenoxy)piperidin-1-yl]pyridazin-3-yl}ethynylphenol

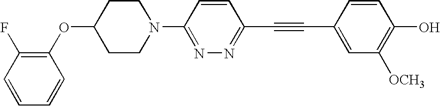

Step 1: 2-Methoxy-4-{6-[4-(2-fluorophenoxy)piperidin-1-yl]pyridazin-3-yl}ethynyl-phenyl acetate: Prepared by Sonogashira coupling reaction of Intermediate 23 with 4-ethynyl-2-methoxyphenyl acetate in a mixture triethylamine and DMSO gave the product as a white solid; IR (KBr) 2955, 2218, 1760, 1499, 1250, 1015, 818 cm⁻¹; ¹H NMR (300 MHz, CDCl₃) δ 1.88-2.10 (m, 4H), 2.32 (s, 3H), 3.61-3.75 (m, 2H), 3.85 (s, 3H), 3.95-4.08 (m, 2H), 4.56 (br s, 1H), 6.82-6.98 (m, 4H), 7.02-7.12 (m, 3H), 7.29 (d, J=9.3 Hz, 1H), 7.42 (d, J=8.4 Hz, 1H); ESI-MS (m/z) 462.41 (M+H)⁺.

Step 2: Deacetylation of Step 1 gave the product as an off-white solid; IR (KBr) 3444, 2948, 2218, 1586, 1504, 1252, 1030, 807, 755 cm⁻¹; ¹H NMR (300 MHz, CDCl₃) δ 1.67 (br s, 2H), 2.01 (br s, 2H), 3.51 (t, J=10.2 Hz, 2H), 3.79 (s, 3H), 4.05 (br s, 2H), 4.68 (br s, 1H), 6.88-7.04 (m, 4H), 7.06-7.26 (m, 4H), 7.50 (d, J=9.3 Hz, 1H), 9.36 (s, 1H); ESI-MS (m/z) 420.32 (M+H)⁺.

### Example 84

### 3-[4-(2-Fluorophenoxy)piperidino]-6-[2-(4-trifluoromethylphenyl)-1-ethynyl]pyridazine

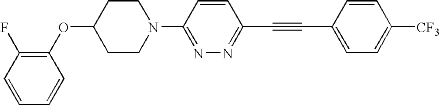

Prepared by coupling reaction of Intermediate 24 with 1-iodo-4-(trifluoromethyl)benzene in a mixture triethylamine and DMSO gave the product as a white solid; IR (KBr) 2948, 2218, 1589, 1323, 1119, 1012, 743 cm⁻¹; ¹H NMR (300 MHz, DMSO-d₆) δ 1.90-2.15 (m, 4H), 3.71-3.78 (m, 2H), 3.97-4.10 (m, 2H), 4.59 (br s, 1H), 6.91 (d, J=9.6 Hz, 1H), 6.97-7.15 (m, 4H), 7.38 (d, J=9.6 Hz, 1H), 7.62 (d, J=8.4 Hz, 2H), 7.69 (d, J=8.4 Hz, 2H).

### Example 85

### 3-(2-{6-[4-(2-Fluorophenoxy)piperidin-1-yl]pyridazin-3-yl}ethynyl])phenyl acetate

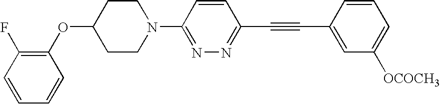

Prepared by Sonogashira coupling reaction of Intermediate 24 with 3-iodophenyl acetate in a mixture of triethylamine and DMSO to give the product as an off-white solid IR (KBr) 2953, 2401, 1772, 1588, 1257, 1010, 743 cm⁻¹; ¹H NMR (300 MHz, CDCl₃) δ 1.84-2.16 (m, 4H), 2.30 (s, 3H), 3.70 (br s, 2H), 4.01 (br s, 2H), 4.57 (br s, 1H), 6.80-6.96 (m, 2H), 7.00-7.18 (m, 4H), 7.20-7.40 (m, 3H), 7.42 (d, J=7.8 Hz, 1H); ESI-MS (m/z) 432.43 (M+H)⁺.

### Example 86

### 3-(2-{6-[4-(2-Fluorophenoxy)piperidino]-3-pyridazinyl}-1-ethynyl)phenol

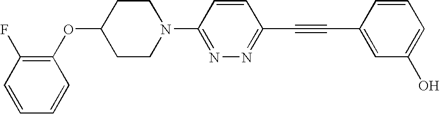

Deacetylation of Example 85 gave the product as an off-white solid; IR (KBr) 3433, 2941, 2211, 1606, 1542, 1450, 1282, 1023, 759 cm⁻¹; ¹H NMR (300 MHz, DMSO-d₆) δ 1.60-1.78 (m, 2H), 1.97-2.16 (m, 2H), 3.50-3.60 (m, 2H), 4.00-4.15 (m, 2H), 4.70 (br s, 1H), 6.85 (d, J=7.2 Hz, 1H), 6.94 (s, 1H), 6.97-7.02 (m, 2H), 7.11-7.35 (m, 5H), 7.58 (d, J=9.6 Hz, 1H), 9.86 (br s, 1H); ESI-MS (m/z) 390.77 (M+H)⁺.

### Example 87

### 3-[{6-[4-(2-Fluorophenoxy)piperidin-1-yl]pyridazin-3-yl}ethynyl]phenyl pivalate

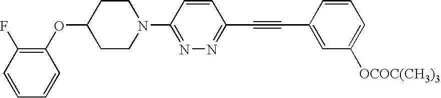

To a stirred solution of Example 86 (300 mg, 0.696 mmol) in dry THF (7 ml) was added triethylamine (105 mg, 1.044 mmol) and pivaloyl chloride (92 mg, 0.765 mmol) under nitrogen atmosphere at room temperature. The reaction mixture was stirred at the same temperature for 12 h. The mixture was diluted with water (50 ml) and extracted with dichloromethane. The organic phase was washed with water (50 ml) and dried over anhydrous sodium sulfate. The crude product obtained after evaporation of the solvent was purified by silica gel column chromatography using 15% EtOAc in chloroform to give 123 mg of the product as an off-white solid; IR (KBr) 2960, 2210, 1755, 1587, 1499, 1258, 1110, 748 cm⁻¹; ¹H NMR (300 MHz, CDCl₃) δ 1.35 (s, 9H), 1.84-2.16 (m, 4H), 3.71 (br s, 2H), 4.01 (br s, 2H), 4.56 (br s, 1H), 6.80-7.00 (m, 2H), 7.02-7.18 (m, 3H), 7.22-7.40 (m, 4H), 7.41 (d, J=7.8 Hz, 1H); ESI-MS (m/z) 474.42 (M+H)⁺.

### Example 88

### 2-(4-{6-[2-(3-Hydroxyphenyl)-1-ethynyl]-3-pyridazinyl}piperazinoxy)benzonitrile

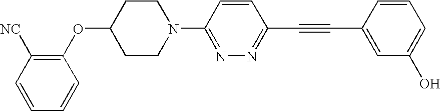

Step 1: 3-(2-{6-[4-(2-Cyanophenoxy)piperidino]-3-pyridazinyl}-1-ethynyl)phenyl acetate: Prepared by Sonogashira coupling reaction of Intermediate 27 with 3-iodophenyl acetate in a mixture of triethylamine and DMSO gave the product as an off-white solid; IR (KBr) 3434, 2924, 2227, 1773, 1594, 1487, 1025, 759 cm⁻¹; ¹H NMR (300 MHz, DMSO-d₆) δ 2.03 (br s, 4H), 2.30 (s, 3H), 3.90 (br s, 4H), 4.76 (br s, 1H), 6.87 (d, J=9.9 Hz, 1H), 6.98-7.10 (m, 4H), 7.22-7.40 (m, 5H).

Step 2: Deacetylation of Step 1 intermediate gave the product as an off-white solid; IR (KBr) 3433, 2941, 2227, 1606, 1450, 1023, 759 cm⁻¹; ¹H NMR (300 MHz, DMSO-d₆) δ 1.76 (br s, 2H), 2.04 (br s, 2H), 3.68 (br s, 2H), 3.98 (br s, 2H), 4.92 (br s, 1H), 6.82 (d, J=7.8 Hz, 1H), 6.92 (s, 1H), 6.98 (d, J=7.5 Hz, 1H), 7.09 (t, J=7.2 Hz, 1H), 7.22 (t, J=7.8 Hz, 1H), 7.32-7.40 (m, 2H), 7.57 (d, J=9.3 Hz, 1H), 7.65 (t, J=8.4 Hz, 1H), 7.72 (d, J=7.2 Hz, 1H), 9.87 (br s, 1H); ESI-MS (m/z) 397.73 (M+H)⁺.

### Example 89

### 3-[2-(3-Fluorophenyl)-1-ethynyl]-6-[4-(2-trifluoromethylphenoxy)piperidino]pyridazine

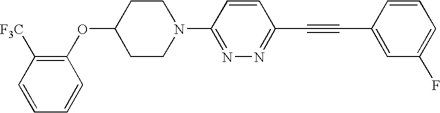

Prepared by Sonogashira coupling reaction of Intermediate 26 with 3-fluoroiodobenzene to give the product as an off-white solid; ¹H NMR (300 MHz, CDCl₃) δ 2.04 (d, J=3.9 Hz, 4H), 3.78-3.85 (m, 2H), 3.96-4.01 (m, 2H), 4.80 (br s, 1H), 6.90 (d, J=9.3 Hz, 1H), 7.02-7.07 (m, 3H), 7.26-7.38 (m, 4H), 7.47 (t, J=8.1 Hz, 1H), 7.61 (d, J=7.2 Hz, 1H); ESI-MS (m/z) 442.60 (M+H)⁺.

### Example 90

### 3-(2-{6-[4-(2-Trifluoromethylphenoxy)piperidino]-3-pyridazinyl}-1-ethynyl)phenyl acetate

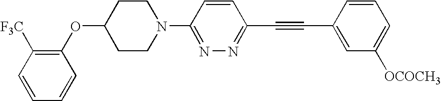

Prepared by Sonogashira coupling reaction of Intermediate 26 with 3-iodophenyl acetate gave the product as an off-white solid; IR (KBr) 2950, 1774, 1588, 1545, 1346, 1269, 1008 cm⁻¹; ¹H NMR (300 MHz, CDCl₃) δ 2.03 (br d, J=4.5 Hz, 4H), 2.32 (s, 3H), 3.80-3.83 (m, 2H), 3.96-4.00 (m, 2H), 4.80 (br s, 1H), 6.89 (d, J=9.3 Hz, 1H), 7.02-7.04 (m, 2H), 7.10 (d, J=8.1 Hz, 1H), 7.26-7.39 (m, 3H), 7.44-7.51 (m, 2H), 7.60 (m, J=7.8 Hz, 1H); ESI-MS (m/z) 482.42 (M+H)⁺.

### Example 91

### 3-(2-{6-[4-(2-Trifluoromethylphenoxy)piperidino]-3-pyridazinyl}-1-ethynyl)phenol

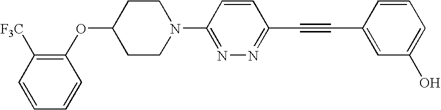

Deacetylation of Example 90 gave the product as an off-white solid; IR (KBr) 3435, 2956, 2218, 1606, 1590, 1442, 1321, 1120, 1036 cm⁻¹; ¹H NMR (300 MHz, DMSO-d₆) δ 1.76 (br s, 2H), 2.00 (br s, 2H), 3.76-3.90 (m, 4H), 4.94 (br s, 1H), 6.82 (d, J=6.9 Hz, 1H), 6.92 (s, 1H), 6.98 (d, J=7.2 Hz, 1H), 7.07 (t, J=7.2 Hz, 1H), 7.19 (t, J=8.4 Hz, 1H), 7.30-7.38 (m, 2H), 7.54-7.62 (m, 3H); ESI-MS (m/z) 440.32 (M+H)⁺.

### Example 92

### 4-[{6-[4-(2,5-Dichlorophenoxy)piperidin-1-yl]pyridazin-3-yl}ethynyl]phenyl acetate

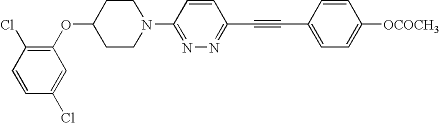

Prepared by a Sonogashira coupling reaction of Intermediate 28 with 4-ethynylphenyl acetate in triethylamine to give the product as an off-white solid; IR (KBr) 2934, 1748, 1581, 1440, 1199, 1032, 916 cm⁻¹; ¹H NMR (300 MHz, CDCl₃) δ 1.98-2.10 (m, 4H), 2.30 (s, 3H), 3.80-4.00 (m, 4H), 4.60-4.70 (m, 1H), 6.84-6.98 (m, 3H), 7.07 (d, J=8.1 Hz, 2H), 7.20-7.38 (m, 2H), 7.56 (d, J=8.1 Hz, 2H); ESI-MS (m/z) 482.32 [100%, (M+H)⁺].

### Example 93

### 4-[{6-[4-(2,5-Dichlorophenoxy)piperidin-1-yl]pyridazin-3-yl}ethynyl]phenol

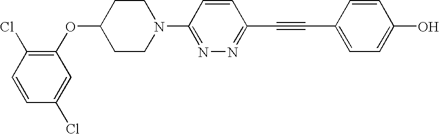

Deacetylation of Example 92 gave the product as an off-white solid; IR (KBr) 3430, 3029, 2935, 2207, 1605, 1581, 1436, 1271, 1023, 928, 803 cm⁻¹; ¹H NMR (300 MHz, DMSO-d₆) δ 2.01 (br s, 4H), 2.30 (s, 1H), 3.86 (br s, 4H), 4.65 (br s, 1H), 6.82-6.98 (m, 3H), 7.07 (d, J=8.1 Hz, 2H), 7.22-7.38 (m, 2H), 7.56 (d, J=8.1 Hz, 2H); ESI-MS (m/z) 441.18 [100%, (M+H)⁺].

### Example 94

### 3-[(2,4-Difluoro-3-methoxyphenyl)ethynyl]-6-[4-(2-fluorophenoxy)piperidin-1-yl]pyridazine

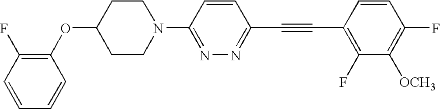

Prepared by a Sonogashira coupling reaction of Intermediate 24 with 2,4-difluoro-3-methoxyiodobenzene in triethylamine to give the product as an off-white solid; IR (KBr) 2941, 1611, 1592, 1258, 1000, 750 cm⁻¹; ¹H NMR (300 MHz, CDCl₃) δ 1.90-2.14 (m, 4H), 3.64-3.80 (m, 2H), 3.92-4.10 (m, 5H), 4.57 (br s, 1H), 6.82-7.18 (m, 7H), 7.35 (d, J=9.6 Hz, 1H).

### Example 95

### 3-[4-(2-Fluorophenoxy)piperidin-1-yl]-6-(1-oxo-pyridin-3-ylethynyl)pyridazine

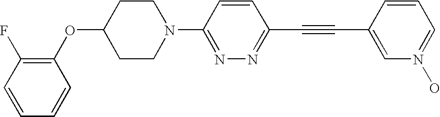

Prepared by a Sonogashira coupling reaction of Intermediate 24 with 3-iodo-1-oxopyridine in triethylamine to give the product as an off-white solid; IR (KBr) 2952, 2220, 1582, 1439, 1260, 1232, 1016, 811, 739 cm⁻¹; ¹H NMR (300 MHz, CDCl₃) δ 2.01-2.21 (m, 4H), 3.74-3.86 (m, 2H), 4.01-4.24 (m, 2H), 4.58 (br s, 1H), 6.90-7.00 (m, 2H), 7.05-7.15 (m, 3H), 7.43 (d, J=9.0 Hz, 2H), 8.16 (d, J=6.0 Hz, 2H), 8.34 (s, 1H); ESI-MS (m/z) 391.44 (M+H)⁺.

### Example 96

### 3-{6-[4-(Pyridin-3-yloxy)1piperidin-1-yl]pyridazin-3-yl}ethynyl benzamide

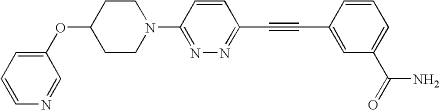

Prepared by Sonogashira coupling reaction of Intermediate 29 with 3-iodobenzamide in a mixture of triethylamine and DMSO to give the product as an off-white solid; IR (KBr) 3376, 2956, 2925, 2850, 1655, 1425, 1233, 1116, 710 cm⁻¹; ¹H NMR (300 MHz, CDCl₃) δ 1.67-1.69 (m, 2H), 2.04 (m, 2H), 3.53 (t, 2H), 4.08 (m, 2H), 4.78 (s, 1H), 7.33-7.35 (m, 2H), 7.50-7.60 (m, 4H), 7.72 (d, J=6.0 Hz, 1H), 7.91 (d, J=9.0 Hz, 1H), 8.09 (br s, 2H), 8.15 (s, 1H), 8.33 (s, 1H); ESI-MS (m/z) 400.60 (M+H)⁺.

### Example 97

### 1-(2-Fluorophenoxy)-4-{5-[2-(3-methylphenyl)-1-ethynyl]-2-pyrimidinyl}piperazine

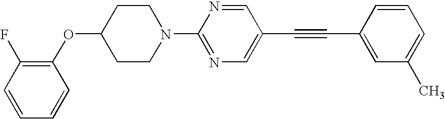

Prepared by Sonogashira coupling reaction of Intermediate 30 with 3-iodotoluene to give the product as a white solid; IR (KBr) 2860, 2203, 1605, 1592, 1517, 1367, 1253, 1034 cm⁻¹; ¹H NMR (300 MHz, CDCl₃) δ 1.87-2.04 (m, 4H), 2.34 (s, 3H), 3.73-3.81 (m, 2H), 4.15-4.23 (m, 2H), 4.54 (br s, 1H), 6.92-7.21 (m, 5H), 7.27-7.32 (m, 3H), 8.41 (s, 2H); ESI-MS (m/z) 388.48 (M+H)⁺.

### Example 98

### 3-(2-[4-(2-Fluorophenoxy)piperidino]-5-pyrimidinyl}-1-ethynyl)phenol

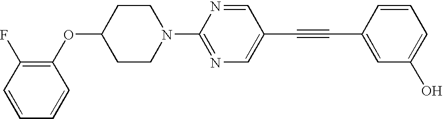

Step 1: 3-(2-[4-(2-Fluorophenoxy)piperidino]-5-pyrimidinyl}-1-ethynyl)phenyl acetate: Prepared by Sonogashira coupling reaction of Intermediate 30 with 3-iodophenyl acetate in a mixture of triethylamine and DMSO to give the product as a white solid; ¹H NMR (300 MHz, CDCl₃) δ 1.85-2.04 (m, 4H), 2.30 (s, 3H), 3.74-3.82 (m, 2H), 4.15-4.22 (m, 2H), 4.52-4.56 (m, 1H), 6.92-7.11 (m, 5H), 7.23 (s, 1H), 7.32-7.35 (m, 2H), 8.40 (s, 2H); ESI-MS (m/z) 432.17 (M+H)⁺.

Step 2: Deacetylation of Step 1 intermediate gave the product as a off-white solid; ¹H NMR (300 MHz, DMSO-d₆) δ 1.85-2.04 (m, 4H), 3.72-3.82 (m, 2H), 4.14-4.22 (m, 2H), 4.54 (br s, 1H), 6.77-6.81 (m, 1H), 6.94 (br s, 2H), 7.03-7.11 (m, 4H), 7.18 (d, J=7.8 Hz, 1H), 8.41 (s, 2H); ESI-MS (m/z) 390.15 (M+H)⁺.

### Example 99

### 4-[(6-{2-[(2-Fluorophenoxy)ethyl]amino}pyridazin-3-yl}ethynyl]phenyl acetate

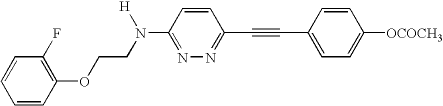

Prepared by Sonogashira coupling reaction of Intermediate 31 with 4-ethynylphenyl acetate in triethylamine to give the product as an off-white solid; IR (KBr) 3228, 2962, 1754, 1610, 1200, 1028, 738 cm⁻¹; ¹H NMR (300 MHz, CDCl₃) δ 2.30 (s, 3H), 3.97 (br s, 2H), 4.27 (br s, 2H), 5.38 (br s, 1H), 6.68 (d, J=9.3 Hz, 1H), 6.82-7.18 (m, 6H), 7.20-7.38 (m, 1H), 7.55 (d, J=8.4 Hz, 2H); ESI-MS (m/z) 392.19 (M+H)⁺.

### Example 100

### 4-[(6-{2-[(2-Fluorophenoxy)ethyl]amino}pyridazin-3-yl}ethynyl]phenol

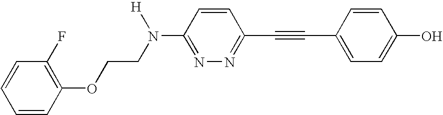

Deacetylation of Example 99 gave the product as an off-white solid; IR (KBr) 3245, 3061, 2950, 2208, 1606, 1277, 752 cm⁻¹; ¹H NMR (300 MHz, DMSO-d₆) δ 3.78 (d, J=5.4 Hz, 2H), 4.23 (t, J=5.4 Hz, 2H), 6.78 (d, J=8.4 Hz, 2H), 6.80-7.00 (m, 2H), 7.10-7.30 (m, 2H), 7.40-7.70 (m, 4H), 9.97 (s, 2H); ESI-MS (m/z) 350.22 (M+H)⁺.

### Example 101

### 4-({6-[4-(2-Fluorophenylamino)piperidin-1-yl]pyridazin-3-yl]pyridazin-3-yl}ethynyl)-phenyl acetate

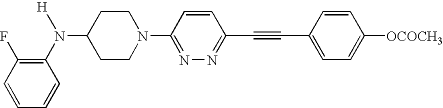

Prepared by Sonogashira coupling reaction of Intermediate 32 with 4-ethnylphenyl acetate in triethylamine to give the product as an off-white solid; IR (KBr) 2953, 1762, 1619, 1431, 1192, 1017, 746 cm⁻¹; ¹H NMR (300 MHz, CDCl₃) δ 1.53 (s, 2H), 2.20 (d, J=13.5 Hz, 2H), 2.31 (s, 3H), 3.23 (t, J=12.6 Hz, 2H), 3.62 (br s, 1H), 3.81 (br s, 1H), 4.39 (d, J=13.5 Hz, 2H), 6.58-6.70 (m, 1H), 6.74 (t, J=8.7 Hz, 1H), 6.86 (d, J=9.6 Hz, 1H), 6.90-7.04 (m, 2H), 7.08 (d, J=8.1 Hz, 2H), 7.33 (d, J=9.3 Hz, 1H), 7.57 (d, J=8.4 Hz, 2H); ESI-MS (m/z) 431.34 (M+H)⁺.

### Example 102

### 4-{6-[4-(2-Fluorophenylamino)piperidin-1-yl]pyridazin-3-yl}ethynylphenol

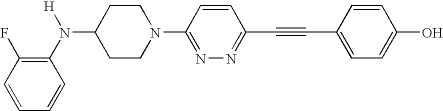

Deacetylation of Example 101 gave the product as an off-white solid; IR (KBr) 3380, 3072, 2933, 2214, 1604, 1282, 750 cm⁻¹; ¹H NMR (300 MHz, CDCl₃) δ 1.48 (br s, 2H), 1.96 (br s, 2H), 3.10 (t, J=12.6 Hz, 2H), 3.50-3.70 (m, 1H), 4.42 (d, J=12.6 Hz, 2H), 5.19 (d, J=6.9 Hz, 1H), 6.44-6.60 (m, 1H), 6.76-6.90 (m, 3H), 6.92-7.04 (m, 2H), 7.27 (d, J=9.3 Hz, 1H), 7.39 (d, J=8.4 Hz, 2H), 7.47 (d, J=9.3 Hz, 1H), 10.00 (br s, 1H, exchangeable with D₂O); ESI-MS (m/z) 389.41 (M+H)⁺.

### In-Vitro Assay of Compounds of the Present Invention

The in-vitro activity of the compounds of the present invention against stearoyl coenzyme desaturase was determined by following conversion of radiolabeled-stearoyl-CoA to oleoyl-CoA using human SCD1 enzyme using a previously published assay procedure with some modifications (Barbara R Talamo and Konrad Bloch, Analytical Biochemistry, 1969, 29, 300-304). This assay protocol is only illustrative and is not meant to limit to the scope of the present invention.

In this assay the microsomal SCD1 enzyme desaturates its substrate, Stearoyl CoA (purchased from American Radiochemicals Ltd.) which is tritiated at C9 and C10 positions. Test compounds were dissolved in dimethylsulfoxide and tested at 10 μM final concentration. Before substrate addition, the test compound or standard reference compound (conjugated linoleic acid at 100 μM final concentration) were pre-incubated in reaction buffer with the enzyme for 10 minutes at 30° C. with shaking. Reaction buffer was prepared as described in literature (Obukowicz et al. JPET, 1998, 287, 157-166) except that the MgCl₂, ATP (purchased from Sigma) and CoA (purchased from Sigma) concentrations were changed to 4.9 mM, 7.2 mM and 0.54 mM respectively. The desaturation reaction was initiated by addition of 0.5 μCi of ³H stearoyl CoA and incubated at 37° C. for 30 minutes with shaking. A control reaction was set without any test compound/reference inhibitor to capture maximum enzymatic activity in the assay. Inhibition of enzyme activity was calculated as a percent of this control reaction giving maximum catalysis and the results are given in Table 5.

Table 5: Tritiated water release assay at 10 μM substrate concentration
*Conjugated Linoleic acid was used as a reference standard

TABLE 5
Tritiated water release assay at 10 μM concentration of test compounds

| Example | % Inhibition |
|---|---|
| Example 1 | 36 |
| Example 2 | 57 |
| Example 3 | 51 |
| Example 4 | 22 |
| Example 5 | 19 |
| Example 6 | 24 |
| Example 7 | 0.35 |
| Example 8 | 42 |
| Example 9 | 86 |
| Example 10 | 37 |
| Example 11 | 63 |
| Example 12 | 11 |
| Example 13 | 92 |
| Example 14 | 35 |
| Example 15 | 49 |
| Example 16 | 42 |
| Example 17 | 40 |
| Example 18 | 26 |
| Example 19 | 14 |
| Example 20 | 38 |
| Example 21 | 40 |
| Example 22 | 84 |
| Example 23 | 91 |
| Example 24 | 79 |
| Example 25 | 97 |
| Example 26 | 27 |
| Example 27 | 30 |
| Example 28 | 98 |
| Example 29 | 24 |
| Example 30 | 26 |
| Example 31 | 21 |
| Example 32 | 35 |
| Example 33 | 20 |
| Example 34 | 29 |
| Example 35 | 36 |
| Example 36 | 23 |
| Example 37 | 89 |
| Example 38 | 99 |
| Example 39 | 75 |
| Example 40 | 81 |
| Example 41 | 93 |
| Example 42 | 92 |
| Example 43 | 98 |
| Example 44 | 37 |
| Example 45 | 43 |
| Example 46 | 29 |
| Example 47 | 33 |
| Example 48 | 22 |
| Example 49 | 91 |
| Example 50 | 66 |
| Example 51 | 22 |
| Example 52 | 39 |
| Example 53 | 47 |
| Example 54 | 47 |
| Example 55 | 60 |
| Example 56 | 92 |
| Example 57 | 96 |
| Example 58 | 6 |
| Example 59 | 94 |
| Example 60 | 95 |
| Example 61 | 97 |
| Example 62 | 99 |
| Example 63 | 94 |
| Example 64 | 87 |
| Example 65 | 93 |
| Example 66 | 94 |
| Example 67 | 96 |
| Example 68 | 92 |
| Example 69 | 93 |
| Example 70 | 96 |
| Example 71 | 95 |
| Example 72 | 38 |
| Example 73 | 37 |
| Example 74 | 44 |
| Example 75 | 88 |
| Example 76 | 100 |
| Example 77 | 97 |
| Example 78 | 43 |
| Example 79 | 37 |
| Example 80 | 86 |
| Example 81 | 95 |
| Example 82 | 98 |
| Example 83 | 93 |
| Example 84 | 37 |
| Example 85 | 97 |
| Example 86 | 100 |
| Example 87 | 27 |
| Example 88 | 95 |
| Example 89 | 82 |
| Example 90 | 95 |
| Example 91 | 100 |
| Example 92 | 97 |
| Example 93 | 96 |
| Example 94 | 12 |
| Example 95 | 24 |
| Example 96 | 30 |
| Example 97 | 36 |
| Example 98 | 95 |
| Example 99 | 55 |
| Example 100 | 63 |
| Example 101 | 82 |
| Example 102 | 96 |
| CLA*(100 μM) | 86 |

*Conjugated Linoleic Acid was used as a reference standard

It is to be understood that any ranges, ratios and ranges or ratios that can be formed by or derived from any of the data disclosed herein represent further embodiments of the present disclosure and are included as part of the disclosure as if they were explicitly set forth.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as described above. All publications, patents, and patent applications cited in this application are herein incorporated by reference to the same extent as if each individual publication, patent, or patent application was specifically and individually indicated to be incorporated herein by reference.

1. A compound of formula I
A-U-B′-≡-Q Formula I
or a pharmaceutically acceptable salt thereof, a solvate thereof, prodrug thereof, stereoisomer thereof, or a N-oxide thereof,
wherein:
A is R′W—;
R′ is selected from substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heteroarylalkyl, substituted or unsubstituted heterocyclic and substituted or unsubstituted heterocyclylalkyl;
W is selected from (CR₁R₂)ₚ, C(═Y), C(═Y)O, OC(═Y), O, CONR₁, S(O)ᵣ, S(O)ᵣNR₁, NR₁(CH₂)ₙO, NR₁ or NR₁C(═Y)NR₂;
Q is selected from hydrogen, hydroxyl, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heteroarylalkyl, substituted or unsubstituted heterocyclic, substituted or unsubstituted heterocyclylalkyl, (CR₁R₂)ₙOR₅, COR₁, COOR₁, CONR₁R₂, S(O)ᵣNR₁R₂, NR₁R₂, (CH₂)ₙNR₁R₂, (CH₂)ₙCHR₁R₂, (CR₁R₂)NR₅R₆, (CR₁R₂)NR₅CONR₆R₇, (CH₂)ₙNHCOR₁ and (CH₂)ₙNHSO₂R₁;
U is selected from
a bond and 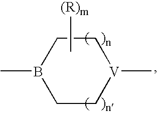
wherein V is CR and N and B is CR or N, or B together with an adjacent ring carbon atom and A form a ring selected from 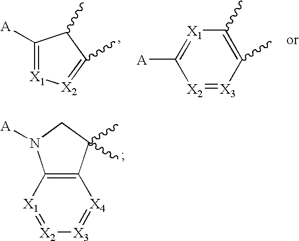
B′ is selected from 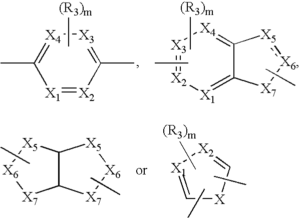
U and V are independently CR or N;
each occurrence of R is independently selected from hydrogen, hydroxyl, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heterocyclyl, substituted or unsubstituted heterocyclylalkyl or substituted or unsubstituted heteroarylalkyl;
each occurrence of m is independently an integer 0-4; each occurrence of n, n′, and r are independently 0, 1 or 2; p is 0, 1, 2, 3 or 4;
each occurrence of R₁, R₂, R₅, R₆, and R₇ may be same or different and are independently hydrogen, halogen, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heterocyclic, substituted or unsubstituted heterocyclylalkyl or substituted or unsubstituted heteroarylalkyl, or when R₁ and R₂ are attached to a common atom, form with the common atom a 3-7 membered heterocyclyl;
each occurrence of X₁ to X₄ are independently N or CR;
each occurrence of X and X₅ to X₇ are independently CHR₄, CO, CS, O, S(O)ᵣ, N or NR₄;
each occurrence of R₄ is independently selected from hydrogen, hydroxyl, substituted or unsubstituted alkyl, substituted or unsubstituted cycloalkyl, substituted or unsubstituted aryl, substituted or unsubstituted heteroaryl and substituted or unsubstituted heterocyclyl;
each occurrence of R₃ is selected from hydrogen, nitro, cyano, halogen, COR₁, substituted or unsubstituted alkyl, substituted or unsubstituted aryl, substituted or unsubstituted heteroaryl, substituted or unsubstituted alkoxy; COOR₁, CONR₁R₂, S(O)ᵣR₁, S(O)ᵣNR₁R₂ and NR₁R₂; and
each occurrence of Y is O or S.

2. The compound according to claim 1, wherein
R′ is selected from substituted or unsubstituted aryl, substituted or unsubstituted heteroaryl and substituted or unsubstituted cycloalkyl;
W is selected from CH₂, CO, O, NH(CH₂)₂O or NH;
B′ is selected from 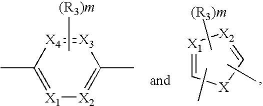
wherein X₁ is N; X₂-X₄ are independently CR or N; X is S; m is an integer 0-4; p is 0, 1, 2, 3 or 4; R₃ is hydrogen
U is selected from bond and 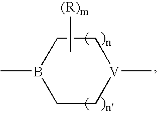
wherein B is CH, C(OH) or N, V is N, R is hydrogen, and n and n′ are independently 0 or 1; and
Q is selected from substituted or unsubstituted alkyl, substituted or unsubstituted cycloalkyl, substituted or unsubstituted cycloalkylalkyl, substituted or unsubstituted aryl, substituted or unsubstituted arylalkyl, substituted or unsubstituted heteroaryl, substituted or unsubstituted heterocyclic, (CR₁R₂)ₙOR₅, (CR₁R₂)ₙNR₅coNR₆R₇, (CH₂)ₙNHCOR₁ and (CH₂)ₙNHSO₂R₁; wherein R₁, R₂, R₅, R₆, and R_{c} independently are selected from hydrogen, substituted or unsubstituted alkyl, substituted or unsubstituted aryl, substituted or unsubstituted heteroaryl and substituted or unsubstituted cycloalkyl.

3. The compound according to claim 1, wherein
R′ is selected from 2-trifluoromethylphenyl, 2,5-dichlorophenyl, 5-trifluoromethylpyridinyl, cyclopentyl, cyclopropyl, cyclohexylmethyl, 2-fluorophenyl, phenyl, 2-fluorophenyl, 4-bromo-2-fluorophenyl, 2-cyanophenyl and 3-pyridyl;
W is selected from CH₂, CO, O, NH(CH₂)₂O or NH;
Q is selected from CH₂OH, C(CH₃)₂OH, substituted or unsubstituted cycloalkyl, C(OH)CH₂CH₃, (CH₂)OR₁, substituted or unsubstituted cycloalkylalkyl, substituted or unsubstituted arylalkyl, (CH₂)ₙNHSO₂R₁, (CH₂)ₙNHCOR₁, (CH₂)₂CH₃, C(CH₃)₃, substituted or unsubstituted aryl, substituted or unsubstituted heteroaryl, and substituted or unsubstituted heterocyclic.

4. A compound selected from:
4-[5-(3-Hydroxy-1-propynyl)-2-pyridyl]piperazino-2-trifluoromethylphenyl methanone,
4-[5-(3-Hydroxy-1-propynyl)-2-pyridyl]piperazino-2,5-dichlorophenylmethanone,
4-[6-(3-Hydroxy-1-propynyl)-3-pyridazinyl]piperazino-2-trifluoromethylphenyl-methanone,
4-[5-(3-Hydroxy-3-methyl-1-butynyl)-2-pyrimidinyl]piperazino-2-trifluoromethylphenyl-methanone,
4-{5-[2-(1-Hydroxycyclopentyl)-1-ethynyl]-2-pyridyl}piperazino-2-trifluoromethyl Phenylmethanone,
4-[5-(3-Hydroxy-1-pentynyl)-2-pyridyl]piperazino-2-trifluoromethylphenyl methanone,
4-[5-{3-Hydroxy-3-(1-adamantyl)-1-propynyl}-2-pyridyl]piperazino-2-trifluoromethyl-phenylmethanone,
4-[5-(3-Hydroxy-3-phenyl-1-propynyl}-2-pyridyl}piperazino-2-trifluoromethylphenyl-methanone,
4-[5-(3-Cyclopentyloxy-1-propynyl)-2-pyridyl]piperazino-2-trifluoromethylphenyl-methanone,
4-{4-[3-(4-tert-Butylphenoxy)-1-propynyl]-2-pyridyl}piperazino-2-trifluoromethyl-phenylmethanone,
4-[5-(3-(4-Fluorophenoxy)-1-propynyl)-2-pyridyl]piperazino-2-trifluoromethylphenyl-methanone,
6-(3-{6-[4-(2-Trifluoromethylbenzoyl)piperazino]-3-pyridyl}-2-propynyloxy)nicotino-nitrile,
4-{5-[3-(4-Hydroxyphenoxy)-1-propynyl]-2-pyridyl}piperazino-2-trifluoromethyl-methanone,
1-{5-[3-(4-Fluorophenoxy)prop-1-yn-1-yl]-2-pyridyl}-4-(5-trifluoromethylpyridin-2-yl)piperazine,
N1-(3-{6-[4-(2-Trifluoromethylbenzoyl)piperazino]-3-pyridyl}-2-propynyl)acetamide,
N1-(3-{6-[4-(2-Trifluoromethylbenzoyl)piperazino]-3-pyridyl}-2-propynyl)-1-butane sulfonamide,
4-[5-(1-Pentynyl)-2-pyridyl]piperazino-2-trifluoromethylphenylmethanone,
4-[5-(3,3-Dimethyl-1-butynyl)-2-pyridyl]piperazino-2-trifluoromethylphenyl methanone,
2,5-Dichlorophenyl-4-[5-(3,3-dimethyl-1-butynyl)-2-pyridyl]piperazinomethanone,
4-[5-(2-Phenyl-1-ethynyl)-2-pyridyl]piperazino-2-trifluoromethylphenylmethanone,
2,5-Dichlorophenyl-4-[5-(2-phenyl-1-ethynyl)-2-pyridyl]piperazinomethanone,
4-(2-{4-[4-(2-Trifluoromethylbenzoyl)piperazino]pyridinyl-1-ethynyl)phenyl acetate,
4-{5-[2-(4-Hydroxyphenyl)-1-ethynyl]-2-pyridyl}piperazino-2-trifluoromethylphenyl methanone,
1-{5-[(3-Fluoro-4-hydroxyphenyl)ethynyl]-2-pyridyl}piperazin-4-yl-(2-trifluoromethylphenyl)methanone,
4-{5-[2-(3-Hydroxyphenyl)-1-ethynyl]-2-pyridyl}piperazino-2-trifluoromethylphenyl Methanone,
Ethyl-2-[3-(2-{6-[4-(2-trifluoromethylbenzoyl)piperazino]-3-pyridyl}-1-ethynyl)-phenoxy]acetate,
2-[3-(2-{6-[4-(2-Trifluoromethylbenzoyl)piperazino]-3-pyridyl}-1-ethynyl)phenoxy]-acetic acid,
2,5-Dichlorophenyl-4-{5-[2-(3-hydroxy-1-pentynyl}-1-ethynyl]-2-pyridyl}piperazino Methanone,
2-(2-{4-[4-(2-Trifluoromethylbenzoyl)piperazino]pyridinyl-1-ethynyl)phenyl acetate,
4-{5-[2-(4-Methoxyphenyl)-1-ethynyl]-2-pyridyl}piperazino-2-trifluoromethylphenyl methanone,
2,5-Dichlorophenyl-4-{5-[2-(3-methoxyphenyl)-1-ethynyl]-2-pyridyl}piperazino-methanone,
Methyl-4-(2-{6-[4-(2-trifluoromethylbenzoyl)piperazino]-2-pyridyl-1-ethynyl)benzoate,
4-{5-[2-(3-Hydroxymethylphenyl)-1-ethynyl]-2-pyridyl}piperazino-2-trifluoromethyl-phenylmethanone,
Ethyl-2-methylcarbonyloxy-5-(2-{6-[4-(2-trifluoromethylbenzoyl)piperazino-3-pyridyl}-1-ethynyl)benzoate,
2-Hydroxy-5-(2-{6-[4-(2-trifluoromethylbenzoyl)piperazino]-3-pyridyl}-1-ethynyl)-benzoic acid,
N1-[3-(2-{6-[4-(2-Trifluoromethylbenzoyl)piperazino]-3-pyridyl}-1-ethynyl)phenyl]Acetamide,
{4-[6-[4-(2-Trifluoromethylbenzoyl)piperazin-1-yl]pyridazin-3-yl]ethynyl}phenol,
4-{6-[2-(3-Hydroxyphenyl)-1-ethynyl]-3-pyridazinyl}piperazino-2-trifluoromethyl-phenylmethanone,
4-{5-[2-(4-Fluorophenyl)-1-ethynyl]-2-pyridyl}piperazino-2-trifluoromethylphenyl methanone,
4-{6-[2-(3,4-Difluorophenyl)-1-ethynyl]-3-pyridazinyl}piperazino-2(trifluoromethyl)-phenylmethanone,
2-Trifluoromethylphenyl-4-{6-[2-(4-trifluoromethylphenyl)-1-ethynyl]-3-pyridazinyl}-piperazinomethanone,
4-{5-[2-(4-Hydroxyphenyl)-1-ethynyl]-2-pyrimidinyl}piperazino-2-trifluoromethyl-phenylmethanone,
4-{5-[2-(3-Hydroxyphenyl)-1-ethynyl]-3-pyrimidinyl}piperazino-2-trifluoromethyl-phenylmethanone,
Ethyl 5-(2-{6-[4-(2-trifluoromethylbenzoyl)piperazino]-2-pyridyl-1-ethynyl)nicotinate,
4-{5-[(2-Pyrazinyl-1-ethynyl)-2-pyridyl]}piperazino-2-trifluoromethylphenyl methanone,
2,5-Dichlorophenyl-4-{5-(2-pyrimidinyl)-1-ethynyl]-2-pyridyl}piperazinomethanone,
4-{5-[2-(1-Butyl-1H-2-imidazolyl)-1-ethynyl]-2-pyridyl}piperazino-2-trifluoromethyl-phenylmethanone,
4-{5-[2-(1-(3-Methylbutyl)-1H-2-imidazolyl)-1-ethynyl]-2-pyridyl}piperazino-2-trifluoro-methylphenylmethanone,
4-{5-[2-(1H-5-Indolyl)-1-ethynyl]-2-pyridyl}piperazino-2-trifluoromethylphenyl-methanone,
4-{5-[2-(1H-5-Indolyl)-1-ethynyl]-2-pyrimidinyl}piperazino-2-trifluoromethylphenyl-methanone,
4-{5-[2-(4-(1,1-Dioxidoisothiazolidine-2-yl)phenyl)-1-ethynyl]-2-pyrimidinyl}piperazino-2-trifluoromethylphenylmethanone,
4-{5-[2-(4-(1H-1-Azolyl)phenyl)-1-ethynyl]-2-pyrimidinyl}piperazino-2-trifluoro-methylphenylmethanone,
4-(2-{2-[4-(2-Trifluoromethylbenzoyl)piperazino]-1,3-thiazol-5-yl}-1-ethynyl)phenyl acetate,
3-({6-[4-(Cyclopentylcarbonyl)piperazin-1-yl]pyridazin-3-yl}ethynyl)benzonitrile,
3-({6-[4-(Cyclopropylmethyl)piperazin-1-yl]pyridazin-3-yl}ethynyl)phenol,
3-([6-{(4-Cyclohexylmethyl)piperazin-1-yl]pyridazin-3-yl}ethynyl)phenyl acetate,
3-({6-[4-(Cyclohexylmethyl)piperazin-1-yl]pyridazin-3-yl}ethynyl)phenol,
3-{4-[(2-Fluorobenzyl)piperazin-1-yl]-6-(tetrahydro-2H-pyran-2-ylethynyl)}pyridazine,
4-[{6-[4-(2-Fluorobenzyl)piperazin-1-yl]pyridazin-3-yl}ethynyl]phenyl acetate,
3-({6-[4-(2-Fluorobenzyl)piperazin-1-yl]pyridazin-3-yl}ethynyl)phenol,
4-{[6-(4-Benzyl-4-hydroxypiperidin-1-yl)pyridazin-3-yl]ethynyl}phenyl acetate,
4-Benzyl-1-{6-[(4-hydroxyphenyl)ethynyl]pyridazin-3-yl}piperidin-4-ol,
4-(2-Fluorobenzyl)-1-{6-[(4-hydroxyphenyl)ethynyl]pyridazin-3-yl}piperidin-4-ol,
4-{[6-(4-Hydroxy-4-[(2,5-dichlorobenzyl)piperidin-1-yl)pyridazin-3-yl]ethynyl}phenyl acetate,
1-{6-[(4-Hydroxyphenyl)ethynyl]pyridazin-3-yl}-4-(2,5-dichlorobenzyl)piperidin-4-ol,
4-[{6-[3-(2-Fluorophenoxyazetidin-1-yl)pyridazin-3-yl}ethynyl]phenyl acetate,
4-[{6-[3-(2-Fluorophenoxyazetidin-1-yl)pyridazin-3-yl}ethynyl]phenol,
3-(2-{6-[(3S)-3-(2-Fluorophenoxy)azolan-1-yl]-3-pyridazinyl}-1-ethynyl)phenyl acetate,
3-(2-{6-[(3S)-3-(2-Fluorophenoxy)azolan-1-yl]-3-pyridyl}-1-ethynyl)phenol,
4-[{6-[(3S)-3-(2-Fluorophenoxy)azolan-1-yl]pyridazin-3-yl}ethynyl]phenylacetate,
4-[{6-[(3S)-3-(2-Fluorophenoxy)azolan-1-yl]pyridazin-3-yl}ethynyl]phenol,
1-[5-(2-Benzo[d][1,3]dioxol-5-yl-1-ethynyl)-2-pyridyl-4-(2-fluorophenoxy)piperidine,
4-(2-Fluorophenoxy)-1-{5-[2-(3-pyridyl)-1-ethynyl]-2-pyridyl}piperidine,
4-(2-Fluorophenoxy)-1-(5-{2-[3-(1-oxo)pyridyl]-1-ethynyl}-2-pyridyl)piperidine,
4-[{6-[4-(2-Fluorophenoxy)piperidin-1-yl]pyridazin-3-yl}ethynyl]phenyl acetate,
4-(2-{6-[4-(2-Fluorophenoxy)piperidino]-3-pyridazinyl}-1-ethynyl)phenol,
4-(2-{6-[4-(2-Fluorophenoxy)piperidin-1-yl]pyridazin-3-yl}ethynyl)phenol potassium,
3-[4-(2-Fluorophenoxy)piperidin-1-yl]-1-{[4-piperidin-1-ylethoxy]phenylethynyl}-pyridazine,
4-{[6-[4-(2-Fluorophenoxy)piperidin-1-yl]pyridazin-3-yl]ethynylphenoxymorpholine,
4-{6-[4-(2-Fluorophenoxy)piperidin-1-yl]pyridazin-3-yl}ethynylphenyl-2-furoate,
4-(2-{6-[4-Bromo-2-fluorophenoxy)piperidino]-3-pyridazinyl}-1-ethynyl)phenol,
2-Fluoro-[4-{6-(4-[2-fluorophenoxy]piperidin-1-yl)pyridazin-3-yl}ethynylphenol,
2-Methoxy-4-{6-[4-(2-fluorophenoxy)piperidin-1-yl]pyridazin-3-yl}ethynylphenol,
3-[4-(2-Fluorophenoxy)piperidino]-6-[2-(4-trifluoromethylphenyl)-1-ethynyl]pyridazine,
3-(2-{6-[4-(2-Fluorophenoxy)piperidin-1-yl]pyridazin-3-yl}ethynyl])phenyl acetate,
3-(2-{6-[4-(2-Fluorophenoxy)piperidino]-3-pyridazinyl}-1-ethynyl)phenol,
3-[{6-[4-(2-Fluorophenoxy)piperidin-1-yl]pyridazin-3-yl}ethynyl]phenyl pivalate,
2-(4-{6-[2-(3-Hydroxyphenyl)-1-ethynyl]-3-pyridazinyl}piperazinoxy)benzonitrile,
3-[2-(3-Fluorophenyl)-1-ethynyl]-6-[4-(2-trifluoromethylphenoxy)piperidino]pyridazine,
3-(2-{6-[4-(2-Trifluoromethylphenoxy)piperidino]-3-pyridazinyl}-1-ethynyl)phenyl acetate,
3-(2-{6-[4-(2-Trifluoromethylphenoxy)piperidino]-3-pyridazinyl}-1-ethynyl)phenol,
4-[{6-[4-(2,5-Dichlorophenoxy)piperidin-1-yl]pyridazin-3-yl}ethynyl]phenyl acetate,
4-[{6-[4-(2,5-Dichlorophenoxy)piperidin-1-yl]pyridazin-3-yl}ethynyl]phenol,
3-[(2,4-Difluoro-3-methoxyphenyl)ethynyl]-6-[4-(2-fluorophenoxy)piperidin-1-yl]Pyridazine,
3-[4-(2-Fluorophenoxy)piperidin-1-yl]-6-(1-oxo-pyridin-3-ylethynyl)pyridazine,
3-{6-[4-(Pyridin-3-yloxy)1piperidin-1-yl]pyridazin-3-yl}ethynyl benzamide,
1-(2-Fluorophenoxy)-4-{5-[2-(3-methylphenyl)-1-ethynyl]-2-pyrimidinyl}piperazine,
3-(2-[4-(2-Fluorophenoxy)piperidino]-5-pyrimidinyl}-1-ethynyl)phenol,
4-[(6-{2-[(2-Fluorophenoxy)ethyl]amino}pyridazin-3-yl}ethynyl]phenyl acetate,
4-[(6-{2-[(2-Fluorophenoxy)ethyl]amino}pyridazin-3-yl}ethynyl]phenol,
4-({6-[4-(2-Fluorophenylamino)piperidin-1-yl]pyridazin-3-yl]pyridazin-3-yl}ethynyl)-phenyl acetate,
4-{6-[4-(2-Fluorophenylamino)piperidin-1-yl]pyridazin-3-yl}ethynylphenol
and pharmaceutically acceptable salts thereof.

5. A pharmaceutical composition comprising a compound of claim 1 and a pharmaceutically acceptable excipient.

6. The pharmaceutical composition of claim 5, further comprising one or more therapeutic agents selected from anti-obesity agents, dipeptidyl peptidase IV (DPP-IV) inhibitors, Protein Tyrosine Phosphatase (PTP-1B) inhibitors and anorectic agents.

7. A method for treating a disease, disorder or syndrome mediated by stearoyl CoA desaturase 1 in a subject in need thereof, comprising administering to the subject a therapeutically effective amount of a compound of claim 1.

8. The method of claim 7, wherein the disease, condition or disorder is selected from obesity, appetite disorder, diabetes, impaired glucose tolerance, insulin resistance, a lipid disorder, metabolic syndrome and fatty liver disease.

9. The method of claim 7, further comprising administering one or more therapeutic agents selected from antiobesity agents, insulin or insulin mimetics, insulin secretagogues, α-glucosidase inhibitors, glucagon receptor antagonists, cholesterol lowering agents, PPARδ agonists, DPP IV inhibitors, dyslipidemic agents, CETP inhibitors, HMG-COA reductase inhibitors, fibrates, guggle lipids and SCD1 inhibitors.

10. The method of claim 7, wherein the disorder is obesity.

11. A process for the preparation of a compound of Formula 4a 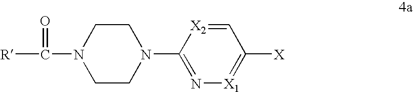
wherein
X is a halogen;
X₁ and X₂ are independently N or CR;
each occurrence of R is independently selected from hydrogen, hydroxyl, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heterocyclyl, substituted or unsubstituted heterocyclylalkyl or substituted or unsubstituted heteroarylalkyl; and
R′ is selected from substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heteroarylalkyl, substituted or unsubstituted heterocyclic and substituted or unsubstituted heterocyclylalkyl,
the method comprising the steps of:
(a) (i) halogenating a compound of formula 1, wherein P is a protecting group, 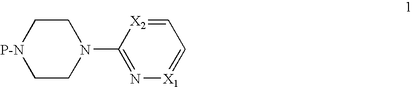
to form a compound of formula 2 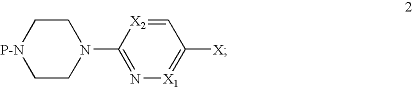
(ii) deprotecting the compound of formula 2 to form an amine; and
(iii) acylating the amine from step (ii) to form a compound of formula 4a, or
(b) (i) deprotecting the compound of formula 1;
(ii) acylating the deprotected compound formed in step (i) to form a compound of formula 3 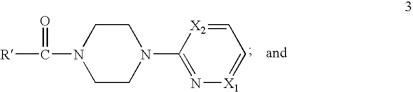
(iii) halogenating the compound of formula 3 to form a compound of formula 4a.

12. A process for the preparation of a compound of Formula 4b 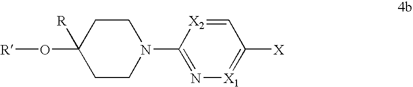
wherein
X is halogen;
X₁ and X₂ are independently N or CR;
each occurrence of R is independently selected from hydrogen, hydroxyl, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heterocyclyl, substituted or unsubstituted heterocyclylalkyl or substituted or unsubstituted heteroarylalkyl; and
R′ is selected from substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heteroarylalkyl, substituted or unsubstituted heterocyclic and substituted or unsubstituted heterocyclylalkyl,
the method comprising the steps of:
(a) reacting a compound of formula 5 with a compound of formula 6 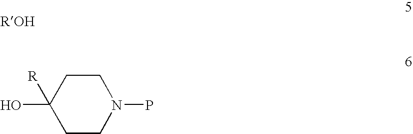
to form a compound of formula 7 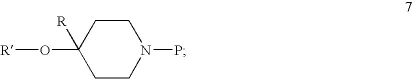
(b) deprotecting the compound of formula 7 to form an amine of formula 8 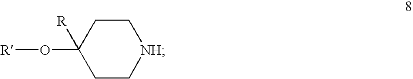
(c) coupling the compound of formula 8 with a compound of formula 9 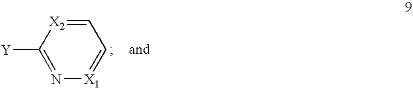
(d) halogenating the product from step (c) to form the compound of formula 4b.

13. A process for the preparation of a compound of Formula 4c 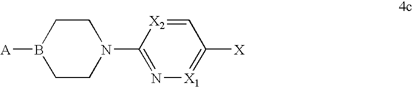
wherein
X is halogen;
A is R′W—;
W is selected from (CR₁R₂)ₚ, C(═Y), C(═Y)O, OC(═Y), O, CONR₁, S(O)ᵣ, S(O)ᵣNR₁, NR₁(CH₂)ₙO, NR₁ or NR₁C(═Y)NR₂;
B is CR or N, or B together with an adjacent ring carbon atom and A form a ring selected from 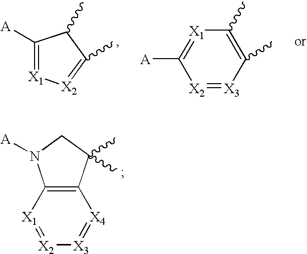
each occurrence of n and r are independently 0, 1 or 2, and p is 0, 1, 2, 3 or 4;
R₁ and R₂ may be same or different and are independently hydrogen, halogen, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heterocyclic, substituted or unsubstituted heterocyclylalkyl or substituted or unsubstituted heteroarylalkyl, or when R₁ and R₂ are attached to a common atom, form with the common atom a 3-7 membered heterocyclyl;
Y is O or S;
each occurrence of X₁ to X₄ are independently N or CR;
each occurrence of R is independently selected from hydrogen, hydroxyl, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heterocyclyl, substituted or unsubstituted heterocyclylalkyl or substituted or unsubstituted heteroarylalkyl; and
R′ is selected from substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heteroarylalkyl, substituted or unsubstituted heterocyclic and substituted or unsubstituted heterocyclylalkyl,
the method comprising the step of:
(a) coupling a compound of formula 10 with a compound of formula 11, where Y in formula 11 is a halogen, 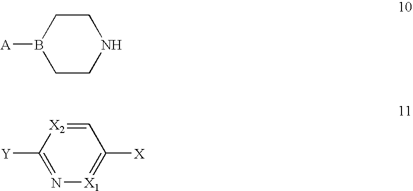
to form a compound of formula 4c.

14. A process for the preparation of a compound of Formula 14 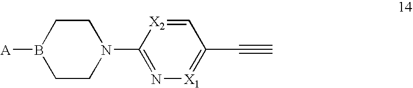
wherein
A is R′W—;
W is selected from (CR₁R₂)ₚ, C(═Y), C(═Y)O, OC(═Y), O, CONR₁, S(O)ᵣ, S(O)ᵣNR₁, NR₁(CH₂)ₙO, NR₁ or NR₁C(═Y)NR₂;
B is CR or N, or B together with an adjacent ring carbon atom and A form a ring selected from 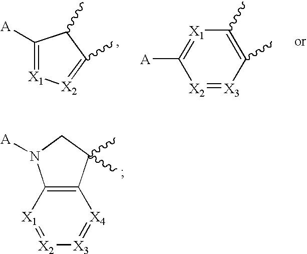
each occurrence of n and r are independently 0, 1 or 2, and p is 0, 1, 2, 3 or 4;
R₁ and R₂ may be same or different and are independently hydrogen, halogen, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heterocyclic, substituted or unsubstituted heterocyclylalkyl or substituted or unsubstituted heteroarylalkyl, or when R₁ and R₂ are attached to a common atom, form with the common atom a 3-7 membered heterocyclyl;
Y is O or S;
each occurrence of X₁ to X₄ are independently N or CR;
each occurrence of R is independently selected from hydrogen, hydroxyl, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heterocyclyl, substituted or unsubstituted heterocyclylalkyl or substituted or unsubstituted heteroarylalkyl; and
R′ is selected from substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heteroarylalkyl, substituted or unsubstituted heterocyclic and substituted or unsubstituted heterocyclylalkyl,
the method comprising the steps of:
(a) (i) coupling a compound of formula 4, where X is a halogen, with a compound of formula 12 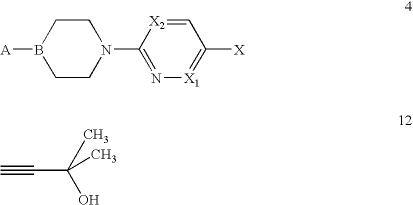
to form a compound of formula 13 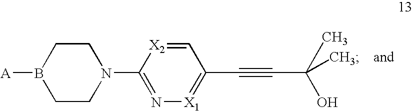
(ii) treating the compound of formula 13 with a base to form a compound of formula 14; or
(b) (i) reacting a compound of formula 4 with a silyl compound of formula 15
≡—Si(CH₃)₃ 15
to form a compound of formula 16 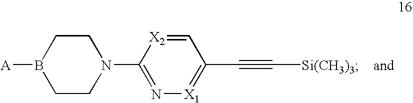
(ii) desilylating the compound of formula 16 to form a compound of formula 14.

15. A process for the preparation of a compound of Formula 1a 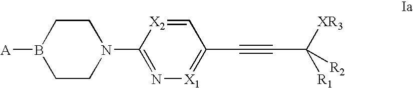
wherein
X is CHR₄, CO, CS, O, S(O)ᵣ, N, NR₄, NHCOR or NHSO₂R;
each occurrence of R₄ is independently selected from hydrogen, hydroxyl, substituted or unsubstituted alkyl, substituted or unsubstituted cycloalkyl, substituted or unsubstituted aryl, substituted or unsubstituted heteroaryl and substituted or unsubstituted heterocyclyl;
R₃ is selected from hydrogen, nitro, cyano, halogen, COR₁, substituted or unsubstituted alkyl, substituted or unsubstituted aryl, substituted or unsubstituted heteroaryl, substituted or unsubstituted alkoxy; COOR₁, CONR₁R₂, S(O)ᵣR₁, S(O)ᵣNR₁R₂ and NR₁R₂;
A is R′W—;
W is selected from (CR₁R₂)ₚ, C(═Y), C(═Y)O, OC(═Y), O, CONR₁, S(O)ᵣ, S(O)ᵣNR₁, NR₁(CH₂)ₙO, NR₁ or NR₁C(═Y)NR₂;
B is CR or N, or B together with an adjacent ring carbon atom and A form a ring selected from 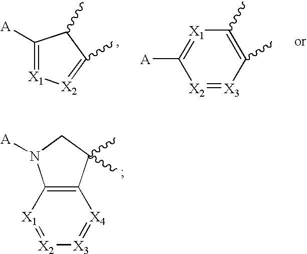
each occurrence of n and r are independently 0, 1 or 2, and p is 0, 1, 2, 3 or 4;
each occurrence of R₁ and R₂ may be same or different and are independently hydrogen, halogen, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heterocyclic, substituted or unsubstituted heterocyclylalkyl or substituted or unsubstituted heteroarylalkyl, or when R₁ and R₂ are attached to a common atom, form with the common atom a 3-7 membered heterocyclyl;
Y is O or S;
each occurrence of X₁ to X₄ are independently N or CR;
each occurrence of R is independently selected from hydrogen, hydroxyl, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heterocyclyl, substituted or unsubstituted heterocyclylalkyl or substituted or unsubstituted heteroarylalkyl; and
R′ is selected from substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heteroarylalkyl, substituted or unsubstituted heterocyclic and substituted or unsubstituted heterocyclylalkyl,
the method comprising the step of:
(a) coupling a compound of formula 4, where X is a leaving group, with a compound of formula 17 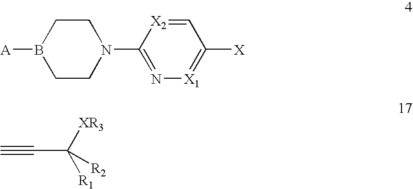
to form a compound of formula Ia.

16. A process for the preparation of a compound of Formula 1b 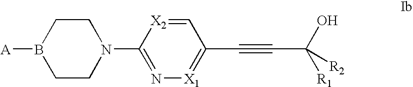
wherein
A is R′W—;
W is selected from (CR₁R₂)ₚ, C(═Y), C(═Y)O, OC(═Y), O, CONR₁, S(O)ᵣ, S(O)ᵣNR₁, NR₁(CH₂)ₙO, NR₁ or NR₁C(═Y)NR₂;
B is CR or N, or B together with an adjacent ring carbon atom and A form a ring selected from 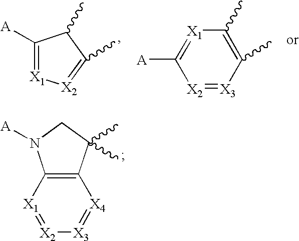
each occurrence of n and r are independently 0, 1 or 2, and p is 0, 1, 2, 3 or 4;
each occurrence of R₁ and R₂ may be same or different and are independently hydrogen, halogen, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heterocyclic, substituted or unsubstituted heterocyclylalkyl or substituted or unsubstituted heteroarylalkyl, or when R₁ and R₂ are attached to a common atom, form with the common atom a 3-7 membered heterocyclyl;
Y is O or S;
each occurrence of X₁ to X₄ are independently N or CR;
each occurrence of R is independently selected from hydrogen, hydroxyl, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heterocyclyl, substituted or unsubstituted heterocyclylalkyl or substituted or unsubstituted heteroarylalkyl; and
R′ is selected from substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heteroarylalkyl, substituted or unsubstituted heterocyclic and substituted or unsubstituted heterocyclylalkyl,
the method comprising the step of:
(a) reacting an intermediate of formula 14 with an intermediate of formula 18 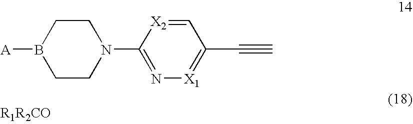
to form a compound of formula Ib.

17. A process for the preparation of a compound of Formula 1c 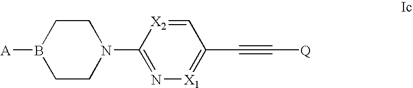
wherein
A is R′W—;
R′ is selected from substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heteroarylalkyl, substituted or unsubstituted heterocyclic and substituted or unsubstituted heterocyclylalkyl;
W is selected from (CR₁R₂)ₚ, C(═Y), C(═Y)O, OC(═Y), O, CONR₁, S(O)ᵣ, S(O)ᵣNR₁, NR₁(CH₂)ₙO, NR₁ or NR₁C(═Y)NR₂;
Q is selected from hydrogen, hydroxyl, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heteroarylalkyl, substituted or unsubstituted heterocyclic, substituted or unsubstituted heterocyclylalkyl, (CR₁R₂)ₙOR₅, COR₁, COOR₁, CONR₁R₂, S(O)ᵣNR₁R₂, NR₁R₂, (CH₂)ₙNR₁R₂, (CH₂)ₙCHR₁R₂, (CR₁R₂)NR₅R₆, (CR₁R₂)NR₅CON₆R₇, (CH₂)ₙNHCOR₁ and (CH₂)ₙNHSO₂R₁;
B is CR or N, or B together with an adjacent ring carbon atom and A form a ring selected from 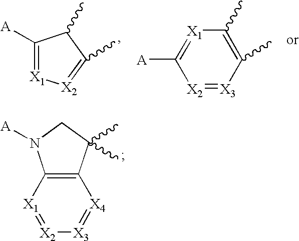
each occurrence of n and r are independently 0, 1 or 2; p is 0, 1, 2, 3 or 4;
each occurrence of R₁, R₂, R₅, R₆, and R₇ may be same or different and are independently hydrogen, halogen, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heterocyclic, substituted or unsubstituted heterocyclylalkyl or substituted or unsubstituted heteroarylalkyl, or when R₁ and R₂ are attached to a common atom, form with the common atom a 3-7 membered heterocyclyl;
each occurrence of X₁ to X₄ are independently N or CR;
each occurrence of R is independently selected from hydrogen, hydroxyl, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heterocyclyl, substituted or unsubstituted heterocyclylalkyl or substituted or unsubstituted heteroarylalkyl; and
Y is O or S,
the method comprising the step of:
(a) coupling of a compound of formula 14 with a compound of formula 19, where X is a leaving group 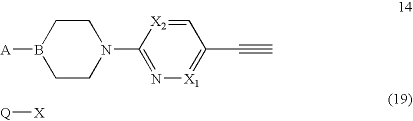
to form a compound of formula Ic.

18. A process for the preparation of a compound of Formula 1c 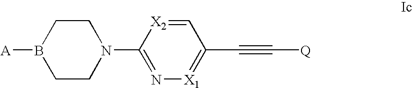
wherein
A is R′W—;
R′ is selected from substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heteroarylalkyl, substituted or unsubstituted heterocyclic and substituted or unsubstituted heterocyclylalkyl;
W is selected from (CR₁R₂)ₚ, C(═Y), C(═Y)O, OC(═Y), O, CONR₁, S(O)ᵣ, S(O)ᵣNR₁, NR₁(CH₂)ₙO, NR₁ or NR₁C(═Y)NR₂;
Q is selected from hydrogen, hydroxyl, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heteroarylalkyl, substituted or unsubstituted heterocyclic, substituted or unsubstituted heterocyclylalkyl, (CR₁R₂)ₙOR₅, COR₁, COOR₁, CONR₁R₂, S(O)ᵣNR₁R₂, NR₁R₂, (CH₂)ₙNR₁R₂, (CH₂)ₙCHR₁R₂, (CR₁R₂)NR₅R₆, (CR₁R₂)NR₅CONR(R₇, (CH₂)ₙNHCOR₁, and (CH₂)ₙNHSO₂R₁;
B is CR or N, or B together with an adjacent ring carbon atom and A form a ring selected from 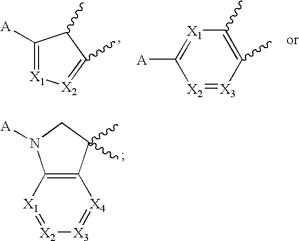
each occurrence of n and r are independently 0, 1 or 2; p is 0, 1, 2, 3 or 4;
each occurrence of R₁, R₂, R₅, R₆, and R₇ may be same or different and are independently hydrogen, halogen, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heterocyclic, substituted or unsubstituted heterocyclylalkyl or substituted or unsubstituted heteroarylalkyl, or when R₁ and R₂ are attached to a common atom, form with the common atom a 3-7 membered heterocyclyl;
each occurrence of X₁ to X₄ are independently N or CR;
each occurrence of R is independently selected from hydrogen, hydroxyl, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heterocyclyl, substituted or unsubstituted heterocyclylalkyl or substituted or unsubstituted heteroarylalkyl; and
Y is O or S,
the method comprising the step of:
(a) coupling of a compound of formula 4, where X is a leaving group, with a compound of formula 20 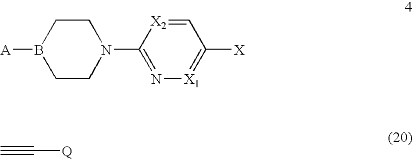
to form a compound of formula Ic.

19. A process for the preparation of a compound of Formula Ic 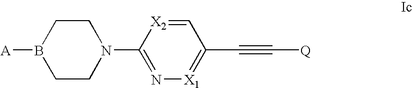
wherein
A is R′W—;
R′ is selected from substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heteroarylalkyl, substituted or unsubstituted heterocyclic and substituted or unsubstituted heterocyclylalkyl;
W is selected from (CR₁R₂)ₚ, C(═Y), C(═Y)O, OC(═Y), O, CONR₁, S(O)ᵣ, S(O)ᵣNR₁, NR₁(CH₂)ₙO, NR₁ or NR₁C(═Y)NR₂;
Q is selected from hydrogen, hydroxyl, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heteroarylalkyl, substituted or unsubstituted heterocyclic, substituted or unsubstituted heterocyclyalkyl, (CR₁R₂)ₙOR₅, COR₁, COOR₁, CONR₁R₂, S(O)ᵣNR₁R₂, NR₁R₂, (CH₂)ₙNR₁R₂, (CH₂)ₙCHR₁R₂, (CR₁R₂)NR₅R₆, (CR₁R₂)NR₅CONR₆R₇, (CH₂)ₙNHCOR₁ and (CH₂)ₙNHSO₂R₁;
B is CR or N, or B together with an adjacent ring carbon atom and A form a ring selected from 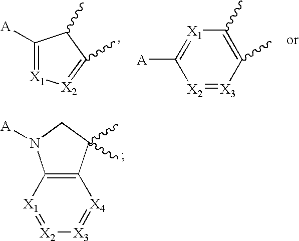
each occurrence of n and r are independently 0, 1 or 2; p is 0, 1, 2, 3 or 4;
each occurrence of R₁, R₂, R₅, R₆, and R₇ may be same or different and are independently hydrogen, halogen, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heterocyclic, substituted or unsubstituted heterocyclylalkyl or substituted or unsubstituted heteroarylalkyl, or when R₁ and R₂ are attached to a common atom, form with the common atom a 3-7 membered heterocyclyl;
each occurrence of X₁ to X₄ are independently N or CR;
each occurrence of R is independently selected from hydrogen, hydroxyl, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heterocyclyl, substituted or unsubstituted heterocyclylalkyl or substituted or unsubstituted heteroarylalkyl; and
Y is O or S,
the method comprising the steps of:
(a) reacting an intermediate of formula 2, wherein P is a protecting group, with a compound of formula 20 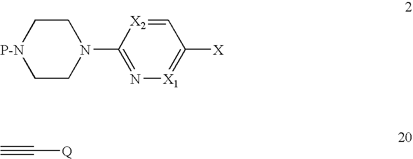
to form a compound of formula 21 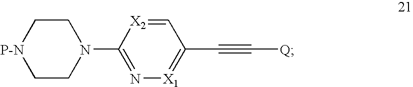
(b) deprotecting the compound of formula 21 to give a compound of formula 22 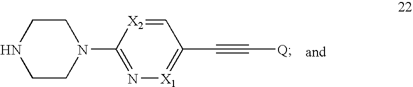
(c) converting the compound of formula 22 to a compound of formula Ic.

20. A method for treating a disease, disorder or syndrome mediated by stearoyl CoA desaturase 1 in a subject in need thereof, comprising administering to the subject a therapeutically effective amount of a compound of claim 4.
